SEQUENCE LISTING (28 sequences)

SEQ ID NO: 1 - DNA, 1629 bp, Homo sapiens
acacatcaggggcttgctcttgcaaaaccaaaccacaagacagacttgcaaaagaaggcatgcacagctcagcactgctctgttgcctggtcctcctgactggggtgagggccagcccaggccagggcacccagtctgagaacagctgcacccacttcccaggcaacctgcctaacatgcttcgagatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtcatcgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaactgagacatcagggtggcgactctatagactctaggacataaattagaggtctccaaaatcggatctggggctctgggatagctgacccagccccttgagaaaccttattgtacctctcttatagaatatttattacctctgatacctcaacccccatttctatttatttactgagcttctctgtgaacgatttagaaagaagcccaatattataatttttttcaatatttattattttcacctgtttttaagctgtttccatagggtgacacactatggtatttgagtgttttaagataaattataagttacataagggaggaaaaaaaatgttctttggggagccaacagaagcttccattccaagcctgaccacgctttctagctgttgagctgttttccctgacctccctctaatttatcttgtctctgggcttggggcttcctaactgctacaaatactcttaggaagagaaaccagggagcccctttgatgattaattcaccttccagtgtctcggagggattcccctaacctcattccccaaccacttcattcttgaaagctgtggccagcttgttatttataacaacctaaatttggttctaggccgggcgcggtggctcacgcctgtaatcccagcactttgggaggctgaggcgggtggatcacttgaggtcaggagttcctaaccagcctggtcaacatggtgaaaccccgtctctactaaaaatacaaaaattagccgggcatggtggcgcgcacctgtaatcccagctacttgggaggctgaggcaagagaattgcttgaacccaggagatggaagttgcagtgagctgatatcatgcccctgtactccagcctgggtgacagagcaagactctgtctcaaaaaataaaaataaaaataaatttggttctaatagaactcagttttaactagaatttattcaattcctctgggaatgttacattgtttgtctgtcttcatagcagattttaattttgaataaataaatgtatcttattcacatc

SEQ ID NO: 2 - protein, 178 aa, Homo sapiens
MHSSALLCCLVLLTGVRASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 3 - DNA, 4507 bp, Homo sapiens
gaccaattgtcatacgacttgcagtgagcgtcaggagcacgtccaggaactcctcagcagcgcctccttcagctccacagccagacgccctcagacagcaaagcctacccccgcgccgcgccctgcccgccgctgcgatgctcgcccgcgccctgctgctgtgcgcggtcctggcgctcagccatacagcaaatccttgctgttcccacccatgtcaaaaccgaggtgtatgtatgagtgtgggatttgaccagtataagtgcgattgtacccggacaggattctatggagaaaactgctcaacaccggaatttttgacaagaataaaattatttctgaaacccactccaaacacagtgcactacatacttacccacttcaagggattttggaacgttgtgaataacattcccttccttcgaaatgcaattatgagttatgtgttgacatccagatcacatttgattgacagtccaccaacttacaatgctgactatggctacaaaagctgggaagccttctctaacctctcctattatactagagcccttcctcctgtgcctgatgattgcccgactcccttgggtgtcaaaggtaaaaagcagcttcctgattcaaatgagattgtggaaaaattgcttctaagaagaaagttcatccctgatccccagggctcaaacatgatgtttgcattctttgcccagcacttcacgcatcagtttttcaagacagatcataagcgagggccagctttcaccaacgggctgggccatggggtggacttaaatcatatttacggtgaaactctggctagacagcgtaaactgcgccttttcaaggatggaaaaatgaaatatcagataattgatggagagatgtatcctcccacagtcaaagatactcaggcagagatgatctaccctcctcaagtccctgagcatctacggtttgctgtggggcaggaggtctttggtctggtgcctggtctgatgatgtatgccacaatctggctgcgggaacacaacagagtatgcgatgtgcttaaacaggagcatcctgaatggggtgatgagcagttgttccagacaagcaggctaatactgataggagagactattaagattgtgattgaagattatgtgcaacacttgagtggctatcacttcaaactgaaatttgacccagaactacttttcaacaaacaattccagtaccaaaatcgtattgctgctgaatttaacaccctctatcactggcatccccttctgcctgacacctttcaaattcatgaccagaaatacaactatcaacagtttatctacaacaactctatattgctggaacatggaattacccagtttgttgaatcattcaccaggcaaattgctggcagggttgctggtggtaggaatgttccacccgcagtacagaaagtatcacaggcttccattgaccagagcaggcagatgaaataccagtcttttaatgagtaccgcaaacgctttatgctgaagccctatgaatcatttgaagaacttacaggagaaaaggaaatgtctgcagagttggaagcactctatggtgacatcgatgctgtggagctgtatcctgcccttctggtagaaaagcctcggccagatgccatctttggtgaaaccatggtagaagttggagcaccattctccttgaaaggacttatgggtaatgttatatgttctcctgcctactggaagccaagcacttttggtggagaagtgggttttcaaatcatcaacactgcctcaattcagtctctcatctgcaataacgtgaagggctgtccctttacttcattcagtgttccagatccagagctcattaaaacagtcaccatcaatgcaagttcttcccgctccggactagatgatatcaatcccacagtactactaaaagaacgttcgactgaactgtagaagtctaatgatcatatttatttatttatatgaaccatgtctattaatttaattatttaataatatttatattaaactccttatgttacttaacatcttctgtaacagaagtcagtactcctgttgcggagaaaggagtcatacttgtgaagacttttatgtcactactctaaagattttgctgttgctgttaagtttggaaaacagtttttattctgttttataaaccagagagaaatgagttttgacgtctttttacttgaatttcaacttatattataagaacgaaagtaaagatgtttgaatacttaaacactgtcacaagatggcaaaatgctgaaagtttttacactgtcgatgtttccaatgcatcttccatgatgcattagaagtaactaatgtttgaaattttaaagtacttttggttatttttctgtcatcaaacaaaaacaggtatcagtgcattattaaatgaatatttaaattagacattaccagtaatttcatgtctactttttaaaatcagcaatgaaacaataatttgaaatttctaaattcatagggtagaatcacctgtaaaagcttgtttgatttcttaaagttattaaacttgtacatataccaaaaagaagctgtcttggatttaaatctgtaaaatcagtagaaattttactacaattgcttgttaaaatattttataagtgatgttcctttttcaccaagagtataaacctttttagtgtgactgttaaaacttccttttaaatcaaaatgccaaatttattaaggtggtggagccactgcagtgttatcttaaaataagaatattttgttgagatattccagaatttgtttatatggctggtaacatgtaaaatctatatcagcaaaagggtctacctttaaaataagcaataacaaagaagaaaaccaaattattgttcaaatttaggtttaaacttttgaagcaaacttttttttatccttgtgcactgcaggcctggtactcagattttgctatgaggttaatgaagtaccaagctgtgcttgaataatgatatgttttctcagattttctgttgtacagtttaatttagcagtccatatcacattgcaaaagtagcaatgacctcataaaatacctcttcaaaatgcttaaattcatttcacacattaattttatctcagtcttgaagccaattcagtaggtgcattggaatcaagcctggctacctgcatgctgttccttttcttttcttcttttagccattttgctaagagacacagtcttctcatcacttcgtttctcctattttgttttactagttttaagatcagagttcactttctttggactctgcctatattttcttacctgaacttttgcaagttttcaggtaaacctcagctcaggactgctatttagctcctcttaagaagattaaaagagaaaaaaaaaggcccttttaaaaatagtatacacttattttaagtgaaaagcagagaattttatttatagctaattttagctatctgtaaccaagatggatgcaaagaggctagtgcctcagagagaactgtacggggtttgtgactggaaaaagttacgttcccattctaattaatgccctttcttatttaaaaacaaaaccaaatgatatctaagtagttctcagcaataataataatgacgataatacttcttttccacatctcattgtcactgacatttaatggtactgtatattacttaatttattgaagattattatttatgtcttattaggacactatggttataaactgtgtttaagcctacaatcattgatttttttttgttatgtcacaatcagtatattttctttggggttacctctctgaatattatgtaaacaatccaaagaaatgattgtattaagatttgtgaataaatttttagaaatctgattggcatattgagatatttaaggttgaatgtttgtccttaggataggcctatgtgctagcccacaaagaatattgtctcattagcctgaatgtgccataagactgaccttttaaaatgttttgagggatctgtggatgcttcgttaatttgttcagccacaatttattgagaaaatattctgtgtcaagcactgtgggttttaatatttttaaatcaaacgctgattacagataatagtatttatataaataattgaaaaaaattttcttttgggaagagggagaaaatgaaataaatatcattaaagataactcaggagaatcttctttacaattttacgtttagaatgtttaaggttaagaaagaaatagtcaatatgcttgtataaaacactgttcactgttttttttaaaaaaaaaacttgatttgttattaacattgatctgctgacaaaacctgggaatttgggttgtgtatgcgaatgtttcagtgcctcagacaaatgtgtatttaacttatgtaaaagataagtctggaaataaatgtctgtttatttttgtactatttaaaaattgacagatcttttctgaagaaaaaaaaaaaaaaa

SEQ ID NO: 4 - protein, 604 aa, Homo sapiens
MLARALLLCAVLALSHTANPCCSHPCQNRGVCMSVGFDQYKCDCTRTGFYGENCSTPEFLTRIKLFLKPTPNTVHYILTHFKGFWNVVNNIPFLRNAIMSYVLTSRSHLIDSPPTYNADYGYKSWEAFSNLSYYTRALPPVPDDCPTPLGVKGKKQLPDSNEIVEKLLLRRKFIPDPQGSNMMFAFFAQHFTHQFFKTDHKRGPAFTNGLGHGVDLNHIYGETLARQRKLRLFKDGKMKYQIIDGEMYPPTVKDTQAEMIYPPQVPEHLRFAVGQEVFGLVPGLMMYATIWLREHNRVCDVLKQEHPEWGDEQLFQTSRLILIGETIKIVIEDYVQHLSGYHFKLKFDPELLFNKQFQYQNRIAAEFNTLYHWHPLLPDTFQIHDQKYNYQQFIYNNSILLEHGITQFVESFTRQIAGRVAGGRNVPPAVQKVSQASIDQSRQMKYQSFNEYRKRFMLKPYESFEELTGEKEMSAELEALYGDIDAVELYPALLVEKPRPDAIFGETMVEVGAPFSLKGLMGNVICSPAYWKPSTFGGEVGFQIINTASIQSLICNNVKGCPFTSFSVPDPELIKTVTINASSSRSGLDDINPTVLLKERSTEL

SEQ ID NO: 5 - DNA, 6082 bp, Homo sapiens
ataacgtctttgtcactaaaatgttccccaggggccttcggcgagtctttttgtttggttttttgtttttaatctgtggctcttgataatttatctagtggttgcctacacctgaaaaacaagacacagtgtttaactatcaacgaaagaactggacggctccccgccgcagtcccactccccgagtttgtggctggcatttgggccacgccgggctgggcggtcacagcgaggggcgcgcagtttggggtcacacagctccgcttctaggccccaaccaccgttaaaaggggaagcccgtgccccatcaggtccgctcttgctgagcccagagccatcccgcgctctgcgggctgggaggcccgggccaggacgcgagtcctgcgcagccgaggttccccagcgccccctgcagccgcgcgtaggcagagacggagcccggccctgcgcctccgcaccacgcccgggaccccacccagcggcccgtacccggagaagcagcgcgagcacccgaagctcccggctggcggcagaaaccgggagtggggccgggcgagtgcgcggcatcccaggccggcccgaacgctccgcccgcggtgggccgacttcccctcctcttccctctctccttcctttagcccgctggcgccggacacgctgcgcctcatctcttggggcgttcttccccgttggccaaccgtcgcatcccgtgcaactttggggtagtggccgtttagtgttgaatgttccccaccgagagcgcatggcttgggaagcgaggcgcgaacccggcccccgaagggccgccgtccgggagacggtgatgctgttgctgtgcctgggggtcccgaccggccgcccctacaacgtggacactgagagcgcgctgctttaccagggcccccacaacacgctgttcggctactcggtcgtgctgcacagccacggggcgaaccgatggctcctagtgggtgcgcccactgccaactggctcgccaacgcttcagtgatcaatcccggggcgatttacagatgcaggatcggaaagaatcccggccagacgtgcgaacagctccagctgggtagccctaatggagaaccttgtggaaagacttgtttggaagagagagacaatcagtggttgggggtcacactttccagacagccaggagaaaatggatccatcgtgacttgtgggcatagatggaaaaatatattttacataaagaatgaaaataagctccccactggtggttgctatggagtgccccctgatttacgaacagaactgagtaaaagaatagctccgtgttatcaagattatgtgaaaaaatttggagaaaattttgcatcatgtcaagctggaatatccagtttttacacaaaggatttaattgtgatgggggccccaggatcatcttactggactggctctctttttgtctacaatataactacaaataaatacaaggcttttttagacaaacaaaatcaagtaaaatttggaagttatttaggatattcagtcggagctggtcattttcggagccagcatactaccgaagtagtcggaggagctcctcaacatgagcagattggtaaggcatatatattcagcattgatgaaaaagaactaaatatcttacatgaaatgaaaggtaaaaagcttggatcgtactttggagcttctgtctgtgctgtggacctcaatgcagatggcttctcagatctgctcgtgggagcacccatgcagagcaccatcagagaggaaggaagagtgtttgtgtacatcaactctggctcgggagcagtaatgaatgcaatggaaacaaacctcgttggaagtgacaaatatgctgcaagatttggggaatctatagttaatcttggcgacattgacaatgatggctttgaagatgttgctatcggagctccacaagaagatgacttgcaaggtgctatttatatttacaatggccgtgcagatgggatctcgtcaaccttctcacagagaattgaaggacttcagatcagcaaatcgttaagtatgtttggacagtctatatcaggacaaattgatgcagataataatggctatgtagatgtagcagttggtgcttttcggtctgattctgctgtcttgctaaggacaagacctgtagtaattgttgacgcttctttaagccaccctgagtcagtaaatagaacgaaatttgactgtgttgaaaatggatggccttctgtgtgcatagatctaacactttgtttctcatataagggcaaggaagttccaggttacattgttttgttttataacatgagtttggatgtgaacagaaaggcagagtctccaccaagattctatttctcttctaatggaacttctgacgtgattacaggaagcatacaggtgtccagcagagaagctaactgtagaacacatcaagcatttatgcggaaagatgtgcgggacatcctcaccccaattcagattgaagctgcttaccaccttggtcctcatgtcatcagtaaacgaagtacagaggaattcccaccacttcagccaattcttcagcagaagaaagaaaaagacataatgaaaaaaacaataaactttgcaaggttttgtgcccatgaaaattgttctgctgatttacaggtttctgcaaagattgggtttttgaagccccatgaaaataaaacatatcttgctgttgggagtatgaagacattgatgttgaatgtgtccttgtttaatgctggagatgatgcatatgaaacgactctacatgtcaaactacccgtgggtctttatttcattaagattttagagctggaagagaagcaaataaactgtgaagtcacagataactctggcgtggtacaacttgactgcagtattggctatatatatgtagatcatctctcaaggatagatattagctttctcctggatgtgagctcactcagcagagcggaagaggacctcagtatcacagtgcatgctacctgtgaaaatgaagaggaaatggacaatctaaagcacagcagagtgactgtagcaatacctttaaaatatgaggttaagctgactgttcatgggtttgtaaacccaacttcatttgtgtatggatcaaatgatgaaaatgagcctgaaacgtgcatggtggagaaaatgaacttaactttccatgttatcaacactggcaatagtatggctcccaatgttagtgtggaaataatggtaccaaattcttttagcccccaaactgataagctgttcaacattttggatgtccagactactactggagaatgccactttgaaaattatcaaagagtgtgtgcattagagcagcaaaagagtgcaatgcagaccttgaaaggcatagtccggttcttgtccaagactgataagaggctattgtactgcataaaagctgatccacattgtttaaatttcttgtgtaattttgggaaaatggaaagtggaaaagaagccagtgttcatatccaactggaaggccggccatccattttagaaatggatgagacttcagcactcaagtttgaaataagagcaacaggttttccagagccaaatccaagagtaattgaactaaacaaggatgagaatgttgcgcatgttctactggaaggactacatcatcaaagacccaaacgttatttcaccatagtgattatttcaagtagcttgctacttggacttattgtacttctgttgatctcatatgttatgtggaaggctggcttctttaaaagacaatacaaatctatcctacaagaagaaaacagaagagacagttggagttatatcaacagtaaaagcaatgatgattaaggacttctttcaaattgagagaatggaaaacagactcaggttgtagtaaagaaatttaaaagacactgtttacaagaaaaaatgaattttgtttggacttcttttactcatgatcttgtgacatattatgtcttcatgcaaggggaaaatctcagcaatgattactctttgagatagaagaactgcaaaggtaataatacagccaaagataatctctcagcttttaaatgggtagagaaacactaaagcattcaatttattcaagaaaagtaagcccttgaagatatcttgaaatgaaagtataactgagttaaattatactggagaagtcttagacttgaaatactacttaccatatgtgcttgcctcagtaaaatgaaccccactgggtgggcagaggttcatttcaaatacatctttgatacttgttcaaaatatgttctttaaaaatataattttttagagagctgttcccaaattttctaacgagtggaccattatcactttaaagccctttatttataatacatttcctacgggctgtgttccaacaaccattttttttcagcagactatgaatattatagtattataggccaaactggcaaacttcagactgaacatgtacactggtttgagcttagtgaaattacttctggataattatttttttataattatggatttcaccatctttctttctgtatatatacatgtgtttttatgtaggtatatatttaccattcttcctatctattcttcctataacacacctttatcaagcatacccaggagtaatcttcaaatcttttgttatattctgaaacaaaagattgtgagtgttgcactttacctgatacacgctgatttagaaaatacagaaaccatacctcactaataactttaaaatcaaagctgtgcaaagactagggggcctatacttcatatgtattatgtactatgtaaaatattgactatcacacaactatttccttggatgtaattctttgttaccctttacaagtataagtgttaccttacatggaaacgaagaaacaaaattcataaatttaaattcataaatttagctgaaagatactgattcaatttgtatacagtgaatataaatgagacgacagcaaaattttcatgaaatgtaaaatatttttatagtttgttcatactatatgaggttctattttaaatgactttctggattttaaaaaatttctttaaatacaatcatttttgtaatatttattttatgcttatgatctagataattgcagaatatcattttatctgactctgccttcataagagagctgtggccgaattttgaacatctgttatagggagtgatcaaattagaaggcaatgtggaaaaacaattctgggaaagatttctttatatgaagtccctgccactagccagccatcctaattgatgaaagttatctgttcacaggcctgcagtgatggtgaggaatgttctgagatttgcgaaggcatttgagtagtgaaatgtaagcacaaaacctcctgaacccagagtgtgtatacacaggaataaactttatgacatttatgtatttttaaaaaactttgtatcgttataaaaaggctagtcattctttcaggagaacatctaggatcatagatgaaaaatcaagccccgatttagaactgtcttctccaggatggtctctaaggaaatttacatttggttctttcctactcagaactactcagaaacaactatatatttcaggttatctgagcacagtgaaagcagagtactatggttgtccaacacaggcctctcagatacaaggggaacacaattacatattgggctagattttgcccagttcaaaatagtatttgttatcaacttactttgttacttgtatcatgaattttaaaaccctaccactttaagaagacagggatgggttattcttttttggcaggtaggctatataactatgtgattttgaaatttaactgctctggattagggagcagtgaatcaaggcagacttatgaaatctgtattatatttgtaacagaatataggaaatttaacataattgatgagctcaaatcctgaaaaatgaaagaatccaaattatttcagaattatctaggttaaatattgatgtattatgatggttgcaaagtttttttgtgtgtccaataaacacattgtaaaaaaaa

SEQ ID NO: 6 - protein, 1032 aa, Homo sapiens [sequence omitted]

SEQ ID NO: 7 - DNA, 2081 bp, Homo sapiens
agcatgagtcagacagcctctggctttctggaagggcaaggactctatatatacagagggagcttcctagctgggatattggagcagcaagaggctgggaagccatcacttaccttgcactgagaaagaagacaaaggccagtatgcacagctttcctccactgctgctgctgctgttctggggtgtggtgtctcacagcttcccagcgactctagaaacacaagagcaagatgtggacttagtccagaaatacctggaaaaatactacaacctgaagaatgatgggaggcaagttgaaaagcggagaaatagtggcccagtggttgaaaaattgaagcaaatgcaggaattctttgggctgaaagtgactgggaaaccagatgctgaaaccctgaaggtgatgaagcagcccagatgtggagtgcctgatgtggctcagtttgtcctcactgaggggaaccctcgctgggagcaaacacatctgacctacaggattgaaaattacacgccagatttgccaagagcagatgtggaccatgccattgagaaagccttccaactctggagtaatgtcacacctctgacattcaccaaggtctctgagggtcaagcagacatcatgatatcttttgtcaggggagatcatcgggacaactctccttttgatggacctggaggaaatcttgctcatgcttttcaaccaggcccaggtattggaggggatgctcattttgatgaagatgaaaggtggaccaacaatttcagagagtacaacttacatcgtgttgcagctcatgaactcggccattctcttggactctcccattctactgatatcggggctttgatgtaccctagctacaccttcagtggtgatgttcagctagctcaggatgacattgatggcatccaagccatatatggacgttcccaaaatcctgtccagcccatcggcccacaaaccccaaaagcgtgtgacagtaagctaacctttgatgctataactacgattcggggagaagtgatgttctttaaagacagattctacatgcgcacaaatcccttctacccggaagttgagctcaatttcatttctgttttctggccacaactgccaaatgggcttgaagctgcttacgaatttgccgacagagatgaagtccggtttttcaaagggaataagtactgggctgttcagggacagaatgtgctacacggataccccaaggacatctacagctcctttggcttccctagaactgtgaagcatatcgatgctgctctttctgaggaaaacactggaaaaacctacttctttgttgctaacaaatactggaggtatgatgaatataaacgatctatggatccaggttatcccaaaatgatagcacatgactttcctggaattggccacaaagttgatgcagttttcatgaaagatggatttttctatttctttcatggaacaagacaatacaaatttgatcctaaaacgaagagaattttgactctccagaaagctaatagctggttcaactgcaggaaaaattgaacattactaatttgaatggaaaacacatggtgtgagtccaaagaaggtgttttcctgaagaactgtctattttctcagtcatttttaacctctagagtcactgatacacagaatataatcttatttatacctcagtttgcatatttttttactatttagaatgtagccctttttgtactgatataatttagttccacaaatggtgggtacaaaaagtcaagtttgtggcttatggattcatataggccagagttgcaaagatcttttccagagtatgcaactctgacgttgatcccagagagcagcttcagtgacaaacatatcctttcaagacagaaagagacaggagacatgagtctttgccggaggaaaagcagctcaagaacacatgtgcagtcactggtgtcaccctggataggcaagggataactcttctaacacaaaataagtgttttatgtttggaataaagtcaaccttgtttctactgttttatacactttcaaaaaaaaaaaaaaaaaaaaaaaaaa

SEQ ID NO: 8 - protein, 469 aa, Homo sapiens
MHSFPPLLLLLFWGVVSHSFPATLETQEQDVDLVQKYLEKYYNLKNDGRQVEKRRNSGPVVEKLKQMQEFFGLKVTGKPDAETLKVMKQPRCGVPDVAQFVLTEGNPRWEQTHLTYRIENYTPDLPRADVDHAIEKAFQLWSNVTPLTFTKVSEGQADIMISFVRGDHRDNSPFDGPGGNLAHAFQPGPGIGGDAHFDEDERWTNNFREYNLHRVAAHELGHSLGLSHSTDIGALMYPSYTFSGDVQLAQDDIDGIQAIYGRSQNPVQPIGPQTPKACDSKLTFDAITTIRGEVMFFKDRFYMRTNPFYPEVELNFISVFWPQLPNGLEAAYEFADRDEVRFFKGNKYWAVQGQNVLHGYPKDIYSSFGFPRTVKHIDAALSEENTGKTYFFVANKYWRYDEYKRSMDPGYPKMIAHDFPGIGHKVDAVFMKDGFFYFFHGTRQYKFDPKTKRILTLQKANSWFNCRKN

SEQ ID NO: 9 - DNA, 1678 bp, Homo sapiens
gagcaaacagagcagcagaaaaggcagttcctcttctccagtgccctccttccctgtctctgcctctccctcccttcctcaggcatcagagcggagacttcagggagaccagagcccagcttgccaggcactgagctagaagccctgccatggcacccctgagaccccttctcatactggccctgctggcatgggttgctctggctgaccaagagtcatgcaagggccgctgcactgagggcttcaacgtggacaagaagtgccagtgtgacgagctctgctcttactaccagagctgctgcacagactatacggctgagtgcaagccccaagtgactcgcggggatgtgttcactatgccggaggatgagtacacggtctatgacgatggcgaggagaaaaacaatgccactgtccatgaacaggtggggggcccctccctgacctctgacctccaggcccagtccaaagggaatcctgagcagacacctgttctgaaacctgaggaagaggcccctgcgcctgaggtgggcgcctctaagcctgaggggatagactcaaggcctgagacccttcatccagggagacctcagcccccagcagaggaggagctgtgcagtgggaagcccttcgacgccttcaccgacctcaagaacggttccctctttgccttccgagggcagtactgctatgaactggacgaaaaggcagtgaggcctgggtaccccaagctcatccgagatgtctggggcatcgagggccccatcgatgccgccttcacccgcatcaactgtcaggggaagacctacctcttcaagggtagtcagtactggcgctttgaggatggtgtcctggaccctgattacccccgaaatatctctgacggcttcgatggcatcccggacaacgtggatgcagccttggccctccctgcccatagctacagtggccgggagcgggtctacttcttcaaggggaaacagtactgggagtaccagttccagcaccagcccagtcaggaggagtgtgaaggcagctccctgtcggctgtgtttgaacactttgccatgatgcagcgggacagctgggaggacatcttcgagcttctcttctggggcagaacctctgctggtaccagacagccccagttcattagccgggactggcacggtgtgccagggcaagtggacgcagccatggctggccgcatctacatctcaggcatggcaccccgcccctccttggccaagaaacaaaggtttaggcatcgcaaccgcaaaggctaccgttcacaacgaggccacagccgtggccgcaaccagaactcccgccggccatcccgcgccacgtggctgtccttgttctccagtgaggagagcaacttgggagccaacaactatgatgactacaggatggactggcttgtgcctgccacctgtgaacccatccagagtgtcttcttcttctctggagacaagtactaccgagtcaatcttcgcacacggcgagtggacactgtggaccctccctacccacgctccatcgctcagtactggctgggctgcccagctcctggccatctgtaggagtcagagcccacatggccgggccctctgtagctccctcctcccatctccttcccccagcccaataaaggtcccttagccccgagtttaaa

SEQ ID NO: 10 - protein, 478 aa, Homo sapiens
MAPLRPLLILALLAWVALADQESCKGRCTEGFNVDKKCQCDELCSYYQSCCTDYTAECKPQVTRGDVFTMPEDEYTVYDDGEEKNNATVHEQVGGPSLTSDLQAQSKGNPEQTPVLKPEEEAPAPEVGASKPEGIDSRPETLHPGRPQPPAEEELCSGKPFDAFTDLKNGSLFAFRGQYCYELDEKAVRPGYPKLIRDVWGIEGPIDAAFTRINCQGKTYLFKGSQYWRFEDGVLDPDYPRNISDGFDGIPDNVDAALALPAHSYSGRERVYFFKGKQYWEYQFQHQPSQEECEGSSLSAVFEHFAMMQRDSWEDIFELLFWGRTSAGTRQPQFISRDWHGVPGQVDAAMAGRIYISGMAPRPSLAKKQRFRHRNRKGYRSQRGHSRGRNQNSRRPSRATWLSLFSSEESNLGANNYDDYRMDWLVPATCEPIQSVFFFSGDKYYRVNLRTRRVDTVDPPYPRSIAQYWLGCPAPGHL

SEQ ID NO: 11 - DNA, 6549 bp, Homo sapiens
gcttggagccgccgcacccgggacggtgcgtagcgctggaagtccggccttccgagagctagctgtccgccgcggcccccgcacgccgggcagccgtccctcgccgcctcgggcgcgccaccatggggccccggctcagcgtctggctgctgctgctgcccgccgcccttctgctccacgaggagcacagccgggccgctgcgaagggtggctgtgctggctctggctgtggcaaatgtgactgccatggagtgaagggacaaaagggtgaaagaggcctcccggggttacaaggtgtcattgggtttcctggaatgcaaggacctgaggggccacagggaccaccaggacaaaagggtgatactggagaaccaggactacctggaacaaaagggacaagaggacctccgggagcatctggctaccctggaaacccaggacttcccggaattcctggccaagacggcccgccaggccccccaggtattccaggatgcaatggcacaaagggggagagagggccgctcgggcctcctggcttgcctggtttcgctggaaatcccggaccaccaggcttaccagggatgaagggtgatccaggtgagatacttggccatgtgcccgggatgctgttgaaaggtgaaagaggatttcccggaatcccagggactccaggcccaccaggactgccagggcttcaaggtcctgttgggcctccaggatttaccggaccaccaggtcccccaggccctcccggccctccaggtgaaaagggacaaatgggcttaagttttcaaggaccaaaaggtgacaagggtgaccaaggggtcagtgggcctccaggagtaccaggacaagctcaagttcaagaaaaaggagacttcgccaccaagggagaaaagggccaaaaaggtgaacctggatttcaggggatgccaggggtcggagagaaaggtgaacccggaaaaccaggacccagaggcaaacccggaaaagatggtgacaaaggggaaaaagggagtcccggttttcctggtgaacccgggtacccaggactcataggccgccagggcccgcagggagaaaagggtgaagcaggtcctcctggcccacctggaattgttataggcacaggacctttgggagaaaaaggagagaggggctaccctggaactccggggccaagaggagagccaggcccaaaaggtttcccaggactaccaggccaacccggacctccaggcctccctgtacctgggcaggctggtgcccctggcttccctggtgaaagaggagaaaaaggtgaccgaggatttcctggtacatctctgccaggaccaagtggaagagatgggctcccgggtcctcctggttcccctgggccccctgggcagcctggctacacaaatggaattgtggaatgtcagcccggacctccaggtgaccagggtcctcctggaattccagggcagccaggatttataggcgaaattggagagaaaggtcaaaaaggagagagttgcctcatctgtgatatagacggatatcgggggcctcccgggccacagggacccccgggagaaataggtttcccagggcagccaggggccaagggcgacagaggtttgcctggcagagatggtgttgcaggagtgccaggccctcaaggtacaccagggctgataggccagccaggagccaagggggagcctggtgagttttatttcgacttgcggctcaaaggtgacaaaggagacccaggctttccaggacagcccggcatgacagggagagcgggttctcctggaagagatggccatccgggtcttcctggccccaagggctcgccgggttctgtaggattgaaaggagagcgtggcccccctggaggagttggattcccaggcagtcgtggtgacaccggcccccctgggcctccaggatatggtcctgctggtcccattggtgacaaaggacaagcaggctttcctggaggccctggatccccaggcctgccaggtccaaagggtgaaccaggaaaaattgttcctttaccaggcccccctggagcagaaggactgccggggtccccaggcttcccaggtccccaaggagaccgaggctttcccggaaccccaggaaggccaggcctgccaggagagaagggcgctgtgggccagccaggcattggatttccagggccccccggccccaaaggtgttgacggcttacctggagacatggggccaccggggactccaggtcgcccgggatttaatggcttacctgggaacccaggtgtgcagggccagaagggagagcctggagttggtctaccgggactcaaaggtttgccaggtcttcccggcattcctggcacacccggggagaaggggagcattggggtaccaggcgttcctggagaacatggagcgatcggaccccctgggcttcaggggatcagaggtgaaccgggacctcctggattgccaggctccgtggggtctccaggagttccaggaataggcccccctggagctaggggtccccctggaggacagggaccaccggggttgtcaggccctcctggaataaaaggagagaagggtttccccggattccctggactggacatgccgggccctaaaggagataaaggggctcaaggactccctggcataacgggacagtcggggctccctggccttcctggacagcagggggctcctgggattcctgggtttccaggttccaagggagaaatgggcgtcatggggacccccgggcagccgggctcaccaggaccagtgggtgctcctggattaccgggtgaaaaaggggaccatggctttccgggctcctcaggacccaggggagaccctggcttgaaaggtgataagggggatgtcggtctccctggcaagcctggctccatggataaggtggacatgggcagcatgaagggccagaaaggagaccaaggagagaaaggacaaattggaccaattggtgagaagggatcccgaggagaccctgggaccccaggagtgcctggaaaggacgggcaggcaggacagcctgggcagccaggacctaaaggtgatccaggtataagtggaaccccaggtgctccaggacttccgggaccaaaaggatctgttggtggaatgggcttgccaggaacacctggagagaaaggtgtgcctggcatccctggcccacaaggttcacctggcttacctggagacaaaggtgcaaaaggagagaaagggcaggcaggcccacctggcataggcatcccagggctgcgaggtgaaaagggagatcaagggatagcgggtttcccaggaagccctggagagaagggagaaaaaggaagcattgggatcccaggaatgccagggtccccaggccttaaagggtctcccgggagtgttggctatccaggaagtcctgggctacctggagaaaaaggtgacaaaggcctcccaggattggatggcatccctggtgtcaaaggagaagcaggtcttcctgggactcctggccccacaggcccagctggccagaaaggggagccaggcagtgatggaatcccggggtcagcaggagagaagggtgaaccaggtctaccaggaagaggattcccagggtttccaggggccaaaggagacaaaggttcaaagggtgaggtgggtttcccaggattagccgggagcccaggaattcctggatccaaaggagagcaaggattcatgggtcctccggggccccagggacagccggggttaccgggatccccaggccatgccacggaggggcccaaaggagaccgcggacctcagggccagcctggcctgccaggacttccgggacccatggggcctccagggcttcctgggattgatggagttaaaggtgacaaaggaaatccaggctggccaggagcacccggtgtcccagggcccaagggagaccctggattccagggcatgcctggtattggtggctctccaggaatcacaggctctaagggtgatatggggcctccaggagttccaggatttcaaggtccaaaaggtcttcctggcctccagggaattaaaggtgatcaaggcgatcaaggcgtcccgggagctaaaggtctcccgggtcctcctggccccccaggtccttacgacatcatcaaaggggagcccgggctccctggtcctgagggccccccagggctgaaagggcttcagggactgccaggcccgaaaggccagcaaggtgttacaggattggtgggtatacctggacctccaggtattcctgggtttgacggtgcccctggccagaaaggagagatgggacctgccgggcctactggtccaagaggatttccaggtccaccaggccccgatgggttgccaggatccatggggcccccaggcaccccatctgttgatcacggcttccttgtgaccaggcatagtcaaacaatagatgacccacagtgtccttctgggaccaaaattctttaccacgggtactctttgctctacgtgcaaggcaatgaacgggcccatggccaggacttgggcacggccggcagctgcctgcgcaagttcagcacaatgcccttcctgttctgcaatattaacaacgtgtgcaactttgcatcacgaaatgactactcgtactggctgtccacccctgagcccatgcccatgtcaatggcacccatcacgggggaaaacataagaccatttattagtaggtgtgctgtgtgtgaggcgcctgccatggtgatggccgtgcacagccagaccattcagatcccaccgtgccccagcgggtggtcctcgctgtggatcggctactcttttgtgatgcacaccagcgctggtgcagaaggctctggccaagccctggcgtcccccggctcctgcctggaggagtttagaagtgcgccattcatcgagtgtcacggccgtgggacctgcaattactacgcaaacgcttacagcttttggctcgccaccatagagaggagcgagatgttcaagaagcctacgccgtccaccttgaaggcaggggagctgcgcacgcacgtcagccgctgccaagtctgtatgagaagaacataatgaagcctgactcagctaatgtcacaacatggtgctacttcttcttctttttgttaacagcaacgaaccctagaaatatatcctgtgtacctcactgtccaatatgaaaaccgtaaagtgccttataggaatttgcgtaactaacacaccctgcttcattgacctctacttgctgaaggagaaaaagacagcgataagctttcaatagtggcataccaaatggcacttttgatgaaataaaatatcaatattttctgcaatccaatgcactgatgtgtgaagtgagaactccatcagaaaaccaaagggtgctaggaggtgtgggtgccttccatactgtttgcccattttcattcttgtattataattaattttctacccccagagataaatgtttgtttatatcactgtctagctgtttcaaaatttaggtcccttggtctgtacaaataatagcaatgtaaaaatggttttttgaacctccaaatggaattacagactcagtagccatatcttccaaccccccagtataaatttctgtctttctgctatgtgtggtactttgcagctgcttttgcagaaatcacaattttcctgtggaataaagatggtccaaaaatagtcaaaaattaaatatatatatatattagtaatttatatagatgtcagcaattaggcagatcaaggtttagtttaacttccactgttaaaataaagcttacatagttttcttcctttgaaagactgtgctgtcctttaacataggtttttaaagactaggatattgaatgtgaaacatccgttttcattgttcacttctaaaccaaaaattatgtgttgccaaaaccaaacccaggttcatgaatatggtgtctattatagtgaaacatgtactttgagcttattgtttttattctgtattaaatattttcagggttttaaacactaatcacaaactgaatgacttgacttcaaaagcaacaaccttaaaggccgtcatttcattagtattcctcattctgcatcctggcttgaaaaacagctctgttgaatcacagtatcagtattttcacacgtaagcacattcgggccatttccgtggtttctcatgagctgtgttcacagacctcagcagggcatcgcatggaccgcaggagggcagattcggaccactaggcctgaaatgacatttcactaaaagtctccaaaacatttctaagactactaaggccttttatgtaatttctttaaatgtgtatttcttaagaattcaaatttgtaataaaactatttgtataaaaattaagcttttattaatttgttgctagtattgccacagacgcattaaaagaaacttactgcacaagctgctaataaatttgtaagctttgcataccttaaaaaaaaaaaaaaaaaa

SEQ ID NO: 12 - protein, 1669 aa, Homo sapiens
MGPRLSVWLLLLPAALLLHEEHSRAAAKGGCAGSGCGKCDCHGVKGQKGERGLPGLQGVIGFPGMQGPEGPQGPPGQKGDTGEPGLPGTKGTRGPPGASGYPGNPGLPGIPGQDGPPGPPGIPGCNGTKGERGPLGPPGLPGFAGNPGPPGLPGMKGDPGEILGHVPGMLLKGERGFPGIPGTPGPPGLPGLQGPVGPPGFTGPPGPPGPPGPPGEKGQMGLSFQGPKGDKGDQGVSGPPGVPGQAQVQEKGDFATKGEKGQKGEPGFQGMPGVGEKGEPGKPGPRGKPGKDGDKGEKGSPGFPGEPGYPGLIGRQGPQGEKGEAGPPGPPGIVIGTGPLGEKGERGYPGTPGPRGEPGPKGFPGLPGQPGPPGLPVPGQAGAPGFPGERGEKGDRGFPGTSLPGPSGRDGLPGPPGSPGPPGQPGYTNGIVECQPGPPGDQGPPGIPGQPGFIGEIGEKGQKGESCLICDIDGYRGPPGPQGPPGEIGFPGQPGAKGDRGLPGRDGVAGVPGPQGTPGLIGQPGAKGEPGEFYFDLRLKGDKGDPGFPGQPGMTGRAGSPGRDGHPGLPGPKGSPGSVGLKGERGPPGGVGFPGSRGDTGPPGPPGYGPAGPIGDKGQAGFPGGPGSPGLPGPKGEPGKIVPLPGPPGAEGLPGSPGFPGPQGDRGFPGTPGRPGLPGEKGAVGQPGIGFPGPPGPKGVDGLPGDMGPPGTPGRPGFNGLPGNPGVQGQKGEPGVGLPGLKGLPGLPGIPGTPGEKGSIGVPGVPGEHGAIGPPGLQGIRGEPGPPGLPGSVGSPGVPGIGPPGARGPPGGQGPPGLSGPPGIKGEKGFPGFPGLDMPGPKGDKGAQGLPGITGQSGLPGLPGQQGAPGIPGFPGSKGEMGVMGTPGQPGSPGPVGAPGLPGEKGDHGFPGSSGPRGDPGLKGDKGDVGLPGKPGSMDKVDMGSMKGQKGDQGEKGQIGPIGEKGSRGDPGTPGVPGKDGQAGQPGQPGPKGDPGISGTPGAPGLPGPKGSVGGMGLPGTPGEKGVPGIPGPQGSPGLPGDKGAKGEKGQAGPPGIGIPGLRGEKGDQGIAGFPGSPGEKGEKGSIGIPGMPGSPGLKGSPGSVGYPGSPGLPGEKGDKGLPGLDGIPGVKGEAGLPGTPGPTGPAGQKGEPGSDGIPGSAGEKGEPGLPGRGFPGFPGAKGDKGSKGEVGFPGLAGSPGIPGSKGEQGFMGPPGPQGQPGLPGSPGHATEGPKGDRGPQGQPGLPGLPGPMGPPGLPGIDGVKGDKGNPGWPGAPGVPGPKGDPGFQGMPGIGGSPGITGSKGDMGPPGVPGFQGPKGLPGLQGIKGDQGDQGVPGAKGLPGPPGPPGPYDIIKGEPGLPGPEGPPGLKGLQGLPGPKGQQGVTGLVGIPGPPGIPGFDGAPGQKGEMGPAGPTGPRGFPGPPGPDGLPGSMGPPGTPSVDHGFLVTRHSQTIDDPQCPSGTKILYHGYSLLYVQGNERAHGQDLGTAGSCLRKFSTMPFLFCNINNVCNFASRNDYSYWLSTPEPMPMSMAPITGENIRPFISRCAVCEAPAMVMAVHSQTIQIPPCPSGWSSLWIGYSFVMHTSAGAEGSGQALASPGSCLEEFRSAPFIECHGRGTCNYYANAYSFWLATIERSEMFKKPTPSTLKAGELRTHVSRCQVCMRRT

SEQ ID NO: 13 - DNA, 8114 bp, Homo sapiens
gggagggacgaaccgcgcgaccgagccctacaaaacccgccccggccgagtggcgaggcgagctttccagccgggctcccagagccgcgctgcgcaggagacgcggtggcctgagagcctgagggtccccggactcgcccaggctctgagcgcgcgcccaccatgagcgcccggaccgcccccaggccgcaggtgctcctgctgccgctcctgctggtgctcctggcggcggcgcccgcagccagcaagggttgtgtctgtaaagacaaaggccagtgcttctgtgacggggccaaaggggagaagggggagaagggctttcctggaccccccggttctcctggccagaaaggattcacaggtcctgaaggcttgcctggaccgcagggacccaagggctttccaggacttccaggactcacgggttccaaaggtgtaaggggaataagtggattgccaggattttctggttctcctggacttccaggcaccccaggcaataccgggccttacggacttgtcggtgtaccaggatgcagtggttctaagggtgagcaggggtttccaggactcccagggacactgggctacccagggatcccgggtgctgctggtttgaaaggacaaaagggtgctcctgctaaagaagaagatatagaacttgatgcaaaaggcgaccccgggttgccaggggctccaggaccccagggtttgccaggccctccaggttttcctgggcctgttggcccacctggtcctccgggattctttggctttccaggagccatgggacctagaggacctaagggtcacatgggtgaaagagtgataggacataaaggagagcggggtgtgaaagggttaacaggacccccgggaccaccaggaacagttattgtgaccctaactggcccagataacagaacggacctcaagggggaaaagggagacaagggagcaatgggcgagcctggacctcctggaccctcaggactgcctggagaatcatatggatctgaaaagggtgctcctggagaccctggcctgcagggaaaacccggaaaagatggtgttcctggcttccctggaagtgagggagtcaagggcaacaggggtttccctgggttaatgggtgaagatggcattaagggacagaaaggggacattggccctccaggatttcgtggtccaacagaatattatgacacataccaggaaaagggagatgaaggcactccaggcccaccagggcccagaggagctcgtggcccacaaggtcccagtggtccccccggagttcctggaagtcctggatcatcaaggcctggcctcagaggagcccctggatggccaggcctgaaaggaagtaaaggggaacgaggccgcccaggaaaggatgccatggggactcctgggtccccaggttgtgctggttcaccaggtcttccaggatcaccgggacctccaggaccgccaggtgacatcgtttttcgcaagggtccacctggagatcacggactgccaggctatctagggtctccaggaatcccaggagttgatgggcccaaaggagaaccaggcctcctgtgtacacagtgcccttatatcccagggcctcccggtctcccaggattgccagggttacatggtgtaaaaggaatcccaggaagacaaggcgcagctggcttgaaaggaagcccagggtccccaggaaatacaggtcttccaggatttccaggtttcccaggtgcccagggtgacccaggacttaaaggagaaaaaggtgaaacacttcagcctgaggggcaagtgggtgtcccaggtgacccggggctcagaggccaacctgggagaaagggcttggatggaattcctggaactccgggagtgaaaggattaccaggacctaaaggcgaactggctctgagtggtgagaaaggggaccaaggtcctccaggggatcctggctcccctgggtccccaggacctgcaggaccagctggaccacctggctacggaccccaaggagaacctggtctccagggcacgcaaggagttcctggagcccccggaccacccggagaagccggccctaggggagagctcagtgtttcaacaccagttccaggcccaccaggacctccagggccccctggccatcctggcccccaaggtccacctggtatccctggatccctggggaaatgtggagatcctggtcttccagggcctgatggtgaaccaggaattccaggaattggatttcctgggcctcctggacctaagggagaccaaggttttccaggtacaaaaggatcactgggttgtcctggaaaaatgggagagcctgggttacctggaaagccaggcctcccaggagccaagggagaaccagcagtagccatgcctggaggaccaggaacaccaggttttccaggagaaagaggcaattctggggaacatggagaaattggactccctggacttccaggtctccctggaactccaggaaatgaagggcttgatggaccacgaggagatccagggcagcctggaccacctggagaacaaggacccccaggaaggtgcatagagggtcccaggggagcccaaggacttccaggcttaaatggattgaaagggcaacaaggcagaagaggtaaaacggggccaaagggagacccaggaattccaggcttggatagatcaggatttcctggagaaactggatcaccaggaattccaggtcatcaaggtgaaatgggaccactgggtcaaagaggatatccaggaaatccgggaattttagggccaccaggtgaagatggagtgattgggatgatgggctttcctggagccattggccctccagggccccctgggaacccaggcacaccagggcagagggggagccctggaattccaggagtaaagggccagagaggaaccccaggagccaagggggaacaaggagataaaggaaatcccgggccttcagagatatcccacgtaataggggacaaaggagaaccaggtctcaaaggattcgcaggaaatccaggtgagaaaggaaacagaggcgttccagggatgccaggtttaaagggcctcaaaggactacccggaccagcaggaccaccaggccccagaggagatttgggcagcactgggaatcctggagaaccaggactgcgtggtataccaggaagcatggggaacatgggcatgccaggttctaaaggaaaaaggggaactttgggattcccaggtcgagcaggaagaccaggcctcccaggtattcatggtctccagggagataagggagagccaggttattcagaaggtacaaggccaggaccaccgggaccaacgggggatccaggactgccgggtgatatgggaaagaaaggagaaatggggcaacctggcccacctggacatttggggcctgctggacctgagggagcccctggaagtcctggaagtcctggcctcccaggaaagccaggtcctcatggtgatttgggttttaaaggaatcaaaggcctcctgggccctccaggaatcagaggccctccaggtcttccaggatttccaggatctcctggaccaatgggtataagaggtgaccaaggacgtgatggaattcctggtccagccggagaaaagggagaaacgggtttattgagggcccctccaggcccaagagggaaccctggtgctcaaggagccaaaggagacaggggagccccaggttttcctggcctcccgggcagaaaaggggccatgggagatgctggacctcgaggacccacaggcatagaaggattcccagggccaccaggtctgcccggtgcaattatccctggccagacaggaaatcgtggtccaccaggctcaagaggaagcccaggtgcgcctggtccccctggacctccagggagtcatgtaataggcataaaaggagacaaagggtctatgggccaccctggcccaaaaggtccacctggaactgcaggagacatgggaccaccaggtcgtctgggagcaccaggtactccaggtcttccaggacccagaggtgatcctggattccaggggtttccaggcgtgaaaggagaaaagggtaatcctggatttctaggatccattggacctccaggaccaattgggccaaaaggaccacctggtgtacgtggagaccctggcacacttaagattatctcccttccaggaagcccagggccacctggcacacctggagaaccagggatgcagggagaacctgggccaccagggccacctggaaacctaggaccctgtgggccaagaggtaagccaggcaaggatggaaaaccaggaactcctggaccagctggagaaaaaggcaacaaaggttctaaaggagagccaggaccagctggatcagatggattgccaggtttgaaaggaaaacgtggagacagtggatcacctgcaacctggacaacgagaggctttgtcttcacccgacacagtcaaaccacagcaattccttcatgtccagaggggacagtgccactctacagtgggttttcttttctttttgtacaaggaaatcaacgagcccacggacaagaccttggaactcttggcagctgcctgcagcgatttaccacaatgccattcttattctgcaatgtcaatgatgtatgtaattttgcatctcgaaatgattattcatactggctgtcaacaccagctctgatgccaatgaacatggctcccattactggcagagcccttgagccttatataagcagatgcactgtttgtgaaggtcctgcgatcgccatagccgttcacagccaaaccactgacattcctccatgtcctcacggctggatttctctctggaaaggattttcattcatcatgttcacaagtgcaggttctgagggcaccgggcaagcactggcctcccctggctcctgcctggaagaattccgagccagcccatttctagaatgtcatggaagaggaacgtgcaactactattcaaattcctacagtttctggctggcttcattaaacccagaaagaatgttcagaaagcctattccatcaactgtgaaagctggggaattagaaaaaataataagtcgctgtcaggtgtgcatgaagaaaagacactgaagctaaaaaagacagcagaactgctatttttcatcctaaagaacaaagtaatgacagaacatgctgttatttaggtatttttctttaaccaaacaatattgctccatgatgacttagtacaaagtttcaatttgtttccccacaaaacaaagcaattctttcaagtcagttctgtgatctgggtctctaatctgtgctgtttcaaagttctctgtggcaaagcagcaactattcacaaaatatcaccaaaaacctattccacttacatccaaggcactgtcactacggtgattgtatgaagtttgaatgctgcaagttatgaaatatttggcccgctggattcccacatttgtcttctttctgtctttaagactcagggaggctaaatcagtgtttgattgccccgccaacccttcctgaaacttcagaccctgggtaggggaagagaagggggcatgtggtatcctggagcattgtgtatagaactggattttcagacctgctgaggaccgtaaggcctgatggaacacagaactgaactgaggttcatggattttccaggactgtttcaaacatgcccattactaacggcaaaagggggattccctgatggaaccataatacccttggaaatactgtatggttttgttttgttttgttggtttttaaagatttttgtttgtttattgaattcatttcactgtagctctaaaatctgcttgtattccaagcatataaaattttcccccttagtgaattagttttaaaatgatattgttatatacatactatgaaatatgtataactttaacttctgttttaccagcatacccacacaaataacaagaatactacttatgaaatgtgcactttatcctcattccataaatgtcggtgcataccttatgtaagggagcagttcaataatccatgaaagaacttaaggcatttgttggtttatcagactcggaatctattttctcattgctctgaatatgtcatcactctaggttttacagatttattcctttgttacttctctaattcttcctttgtaaaaaaaaaaaaaagcaacactttttatgttatatgttgttcttacaaaccatactgaaagagtccattgtttaaaaatcttaatgtatcaaactgtataacttggccgctgtatgtcttaaaacctgcttttcaatgtgttgatacattcccaaggttacttaattcaacttaactatcatcttattcagcaccaagcatgtcccaggcactgtactaacctacagagatgctaagagaaaaaaaagacttgtttctgatctaatatcccagaaaaagtaactcattgctctgttaataatctcacatatacaagtagcttccctcccctctagttttttcttccttttcactgctgttatatttcatcatgataattcagcaggcccaagtaaaggttaaaaataaggtctatgcctagggaaactcagggcttctagtttctcttagaaaagctaagagaagataaggtctgaataatagcagaaaaaccaacatctacaaaacattaaactagtgttatacttgatgataacactatttgatgagtcttagagtccagacacaaagagacaaagctttgaagatgctttttgatctacctaggtggagttggtggtgctgatatttaaattcaggctactgcttcaatctcaattgctttgtaagtgaaaaacatgacccagaggacagcacagactatggccatggctcacatggtttacatccttcactgctcacgtgtttgctgtcaagccatttttacatctaaactaagatgtgcagcatttcacttatttagattcacttaacaaacaaatttttctgctttaaaaatgtcttattgtcccaagtgtactatagcggcatatagagctagctaatctctacaaaccctctgtaggccagtagttctcaaagtgtggtctctggaagagcagtatcagcatcatctgggaacttgtcacagatgcagattctagggaccactccagacctacacaatcagaaactcttgggggagggcccgaaatatctatgttttaccaagcccaccacatgattctgatgtactctaaatactgagaaaacctgttctagacaaatacccaagcaacaactccgcaggcagttaccaagtacggctggctacaactgctccatccgtgcctctttttaaagttcaaactcacaggtgactctaaggttatctacttttactcataagtaaaagccctagactggtgctaatgtcaaaccactggcctccactcaggcctccatcttctcatgccctcttaccagtatttaacttctgaggaagacaagtgatgctaaaacctgaaattccaatgaagccatatgaacagctgttcagttgcacttctaagactttacttagcagtaaattatagctcatgtgcattattttccagataacttagcttatgagtagcttatacaattatgaagatttaatattacagataaaatgtaaactgtttctttaaaattggggcttcaactttggaatttcacagcgtgctaaaataacagatttctcagaagtctttcagcaagataaacattattaagtaacttatttatgaaagtattaaaatgcttacatttgaacttgatggctaacttacaaagattctctatgtatcaaatgtaacttactgcgactaaacttaatttaatatttactctataaccaaatgaaatatatttaaaatatattgaatattttatattgttatatcctgacaagattataatattttaatgtactaatatttctgtaattatatctaaaatattattttattatattgcctaagaataaacatttgttaaattggaaaaaaaaaaaaaaaaaa

SEQ ID NO: 14 - protein, 1670 aa, Homo sapiens
MSARTAPRPQVLLLPLLLVLLAAAPAASKGCVCKDKGQCFCDGAKGEKGEKGFPGPPGSPGQKGFTGPEGLPGPQGPKGFPGLPGLTGSKGVRGISGLPGFSGSPGLPGTPGNTGPYGLVGVPGCSGSKGEQGFPGLPGTLGYPGIPGAAGLKGQKGAPAKEEDIELDAKGDPGLPGAPGPQGLPGPPGFPGPVGPPGPPGFFGFPGAMGPRGPKGHMGERVIGHKGERGVKGLTGPPGPPGTVIVTLTGPDNRTDLKGEKGDKGAMGEPGPPGPSGLPGESYGSEKGAPGDPGLQGKPGKDGVPGFPGSEGVKGNRGFPGLMGEDGIKGQKGDIGPPGFRGPTEYYDTYQEKGDEGTPGPPGPRGARGPQGPSGPPGVPGSPGSSRPGLRGAPGWPGLKGSKGERGRPGKDAMGTPGSPGCAGSPGLPGSPGPPGPPGDIVFRKGPPGDHGLPGYLGSPGIPGVDGPKGEPGLLCTQCPYIPGPPGLPGLPGLHGVKGIPGRQGAAGLKGSPGSPGNTGLPGFPGFPGAQGDPGLKGEKGETLQPEGQVGVPGDPGLRGQPGRKGLDGIPGTPGVKGLPGPKGELALSGEKGDQGPPGDPGSPGSPGPAGPAGPPGYGPQGEPGLQGTQGVPGAPGPPGEAGPRGELSVSTPVPGPPGPPGPPGHPGPQGPPGIPGSLGKCGDPGLPGPDGEPGIPGIGFPGPPGPKGDQGFPGTKGSLGCPGKMGEPGLPGKPGLPGAKGEPAVAMPGGPGTPGFPGERGNSGEHGEIGLPGLPGLPGTPGNEGLDGPRGDPGQPGPPGEQGPPGRCIEGPRGAQGLPGLNGLKGQQGRRGKTGPKGDPGIPGLDRSGFPGETGSPGIPGHQGEMGPLGQRGYPGNPGILGPPGEDGVIGMMGFPGAIGPPGPPGNPGTPGQRGSPGIPGVKGQRGTPGAKGEQGDKGNPGPSEISHVIGDKGEPGLKGFAGNPGEKGNRGVPGMPGLKGLKGLPGPAGPPGPRGDLGSTGNPGEPGLRGIPGSMGNMGMPGSKGKRGTLGFPGRAGRPGLPGIHGLQGDKGEPGYSEGTRPGPPGPTGDPGLPGDMGKKGEMGQPGPPGHLGPAGPEGAPGSPGSPGLPGKPGPHGDLGFKGIKGLLGPPGIRGPPGLPGFPGSPGPMGIRGDQGRDGIPGPAGEKGETGLLRAPPGPRGNPGAQGAKGDRGAPGFPGLPGRKGAMGDAGPRGPTGIEGFPGPPGLPGAIIPGQTGNRGPPGSRGSPGAPGPPGPPGSHVIGIKGDKGSMGHPGPKGPPGTAGDMGPPGRLGAPGTPGLPGPRGDPGFQGFPGVKGEKGNPGFLGSIGPPGPIGPKGPPGVRGDPGTLKIISLPGSPGPPGTPGEPGMQGEPGPPGPPGNLGPCGPRGKPGKDGKPGTPGPAGEKGNKGSKGEPGPAGSDGLPGLKGKRGDSGSPATWTTRGFVFTRHSQTTAIPSCPEGTVPLYSGFSFLFVQGNQRAHGQDLGTLGSCLQRFTTMPFLFCNVNDVCNFASRNDYSYWLSTPALMPMNMAPITGRALEPYISRCTVCEGPAIAIAVHSQTTDIPPCPHGWISLWKGFSFIMFTSAGSEGTGQALASPGSCLEEFRASPFLECHGRGTCNYYSNSYSFWLASLNPERMFRKPIPSTVKAGELEKIISRCQVCMKKRH

SEQ ID NO: 15 - DNA, 6192 bp, Homo sapiens
gcgagtgactgcaccgagcccgagaagtcgccgcgccccgcagccgccccgactggttccccgccttgcccgtgggccccgccgggatggggaaccgccgggacctgggccagccgcgggccggtctctgcctgctcctggccgcgctgcagcttctgccggggacgcaggccgatcctgtggatgtcctgaaggccctgggtgtgcagggaggccaggctggggtccccgaggggcctggcttctgtccccagaggactccagagggtgaccgggcattcagaattggccaggccagcacgctcggcatccccacgtgggaactctttccagaaggccactttcctgagaacttctccttgctgatcaccttgcggggacagccagccaatcagtctgtcctgctgtccatttatgatgaaaggggtgcccggcagttgggcctggcactggggccagcgctgggtctcctaggtgaccccttccgccccctcccccagcaggtcaacctcacagatggcaggtggcaccgtgtggccgtcagcatagatggtgagatggtgaccctggtagctgactgtgaagctcagccccctgttttgggccatggcccccgcttcatcagcatagctggactcactgtgctggggacccaggaccttggggaaaagactttcgagggagacattcaggagctgctgataagcccagatcctcaggctgccttccaggcttgtgagcggtacctccccgactgtgacaacctggcaccggcagccacagtggctccccagggtgaaccagaaacccctcgtcctcggcggaaggggaagggaaaagggaggaagaaagggcgaggtcgcaaggggaagggcaggaaaaagaacaaggaaatttggacctcaagtccacctcctgactccgcagagaaccagacctccactgacatccccaagacagagactccagctccaaatctgcctccgacccccacgcctttggtcgtcacctccactgtgactactggactcaatgccacgatcctagagaggagcttggaccctgacagtggaaccgagctggggaccctggagaccaaggcagccagggaggatgaagaaggagatgattccaccatgggccctgacttccgggcagcagaatatccatctcggactcagttccagatctttcctggtgctggagagaaaggagcaaaaggagagcccgcagtgattgaaaaggggcagcagtttgagggacctccaggagccccaggaccccaaggggtggttggcccctcaggccctcccggccccccaggattccctggcgaccctggtccaccgggccctgctggcctcccaggaatccccggcattgatgggatccgaggcccaccgggcactgtgatcatgatgccgttccagtttgcaggcggctcctttaaaggccccccagtctcattccagcaggcccaggctcaggcagttctgcagcagactcagctctctatgaaaggcccccctggtccagtggggctcactgggcgcccaggccctgtgggtctccccgggcatccaggtctgaaaggagaggagggagcagaagggccacagggtccccgaggcctgcagggacctcatggaccccctggccgagtgggcaagatgggccgccctggagcagatggagctcggggcctcccaggggacactggacctaagggtgatcgtggcttcgatggcctccctgggctgcctggtgagaagggccaaaggggtgactttggccatgtggggcaacccggtcccccaggagaggatggtgagaggggagcagagggacctccagggcccactggccaggctggggagccgggtccacgaggactgcttggccccagaggctctcctggccccacgggtcgcccgggtgtgactggaattgatggtgctcctggtgccaaaggcaatgtgggtcctccaggagaaccaggccctccgggacagcagggaaaccatgggtcccagggactccccggtccccagggactcattggcactcctggggagaagggtccccctggaaacccaggaattccaggcctcccaggatccgatggccctctgggtcacccaggacatgagggccccacgggagagaaaggggctcagggtccaccagggtcggcaggccctccgggctatcctggacctcggggagtgaagggcacttcaggcaaccggggcctccagggggagaaaggcgagaagggagaggacggcttcccaggcttcaagggcgatgtggggctcaaaggtgatcaggggaaacccggagctccaggtccccggggagaggatggtcctgaggggccgaaggggcaggcggggcaggctggcgaggaggggcccccaggctcagctggggagaagggcaagcttggggtgccaggcctcccaggttatccaggacgccctggacctaagggatctattggatttcccggtcccctgggacccataggagagaaagggaagtcgggaaagacagggcagccaggcctggaaggagagcggggaccaccaggttcccgtggagagagggggcaaccgggtgccacagggcaaccaggccccaagggcgatgtgggccaggatggagcccctgggatccctggagaaaagggcctccctggtctgcaaggccctccaggattccctgggccaaagggcccccctggtcaccaaggtaaagatgggcgaccagggcaccctggacagagaggagaactgggcttccaaggtcagacaggcccgcctggaccagctggtgtcttaggccctcagggaaagacaggagaagtgggacctctaggtgaaagggggcctccaggcccccctggacctcctggtgaacaaggtcttcctggcctggaaggcagagagggggccaagggggaactgggaccaccaggaccccttgggaaagaagggccagctggactcaggggctttcccggccccaaagggggccctggggacccgggacctactggcttaaagggtgataagggccccccagggcccgtgggggccaatggctcccctggtgagcgcggtcctttgggcccagcaggaggcattggacttcctggccaaagtggcagcgaaggccccgttggccctgcaggcaagaaggggtcccggggagaacgtggcccccctggccccactggcaaagatgggatcccagggcccctggggcctctgggaccccctggagctgctgggccttctggcgaggaaggggacaagggggatgtgggtgcccccggacacaaggggagtaaaggcgataaaggagacgcgggcccacctggacaaccagggatacggggtcctgcaggacacccaggtcccccgggagcagacggggctcaggggcgccggggacccccaggcctctttgggcagaaaggagatgacggagtcagaggctttgtgggggtgattggccctcctggactgcaggggctgccaggccctccgggagagaaaggggaggtcggagacgtcgggtccatgggtccccatggagctccaggtcctcggggtccccaaggccccactggatcagagggcactccagggctgcctggaggagttggtcagccaggcgccgtgggtgagaagggtgagcgaggggacgctggagacccagggcctccaggagccccaggcatcccggggcccaagggagacattggtgaaaagggggactcaggcccatctggagctgctggacccccaggcaagaaaggtccccctggagaggatggagccaaagggagcgtgggccccacggggctgcccggagatctagggcccccaggagaccctggagtttcaggcatagatggttccccaggggagaagggagaccctggtgatgttgggggaccgggtccgcctggagcttctggggagcccggcgcccccgggccccccggcaagaggggtccttcaggccacatgggtcgagaaggcagagaaggggagaaaggtgccaagggggagccaggtcctgatgggcccccagggaggacgggtccaatgggggctagagggccccctggacgtgtggggcctgagggtcttcgagggatccctggccctgtgggtgaaccaggcctcctgggagcccctggacagatgggccctcctggccccctggggccctctggcctcccagggctgaagggagacactggccccaagggggaaaagggccacattggattgatcggtctcattggccccccgggagaagctggtgagaaaggagatcaggggttgccaggcgtgcagggaccccctggtcccaagggagaccctggtccccctggtcccattggctctctgggccaccctgggcccccaggtgtggcgggccctctaggacagaaaggctcaaaagggtctccggggtccatgggcccccgtggagacactggacctgcaggcccaccaggccccccgggtgcccctgccgagctgcatgggctgcgcaggcgccggcgcttcgtcccagtcccgcttccagtcgtggagggcggcctggaggaggtgctggcctcgctcacatcgctgagcttggagctggagcagctgcggcgtcctcccggcactgcggagcgcccgggcctcgtgtgccacgagctgcaccgcaaccacccgcacctgcctgatggggaatactggattgaccccaaccagggctgcgcgcgggactcgttcagggttttttgcaacttcacggcgggaggagagacctgcctctatcccgacaagaagtttgagatcgtgaaattggcctcctggtccaaggaaaagcctggaggctggtatagcacattccgtcgagggaagaagttctcctacgtggacgccgacgggtccccagtgaatgtcgtgcagctgaacttcctgaaactgctgagtgccacagctcgccagaacttcacctactcctgccagaatgcagctgcctggctggacgaagccacgggtgactacagccactccgcccgcttccttggcaccaatggagaggagctgtctttcaaccagacgacagcagccactgtcagcgtcccccaggatggctgccggctccggaaaggacagacgaagacccttttcgaattcagctcttctcgagcgggatttctgcccctgtgggatgtggcggccactgactttggccagacgaaccaaaagtttgggtttgaactgggccccgtctgcttcagcagctgagagtgtccggggtgggagggaccatgagggagccccagaatggggtgcatttggtgctgaggctttgaagccaccgtatttttcgttacctgtgactatggagccaatgggatgtgacttcgctcatcacggacagtcattccttctcctttccagggtgctgggggctggggttccctggcccaagggtccagcctcctctcaccccattccaggtggcatactgcagtctggctctttctcccctccctccccacccaagcctcacctccccaccccttgaacccccatgcaatgagcttctaactcagagctgatgaacaaaagcccccccacccccaatgcctgcctcctcactcctccgtcgctgcccttcacaccttttggtgctacccctccccagagttaagcactggatgtctcctgatcccaggctgggacccctacccccaccccctttgatcctttctacttccacggtgaaaggactgaggtcggactacagagggaagagggacttcccttgactgggttgtgtttcttttcctgcctcagcccagctctgcaaatcccctccccctgccccccacctccccaggctcaccttgccatgccaggtggtttggggaccaagatgttgggggggtgaatcaggatcctaatggtgctgccctatttatacctgggtctgtattaaaagggaaagtcccccctgttgtagatttcatctgcttcctccttagggaaggctgggatatgatgagagattccagcccaagcctggccccccaccgccaggccatagggcataatttgcatctcaaatctgagaataaactgatgaactgtgaaaaaaaaaaaaaaaaaa

SEQ ID NO: 16 - protein, 1745 aa, Homo sapiens
MGNRRDLGQPRAGLCLLLAALQLLPGTQADPVDVLKALGVQGGQAGVPEGPGFCPQRTPEGDRAFRIGQASTLGIPTWELFPEGHFPENFSLLITLRGQPANQSVLLSIYDERGARQLGLALGPALGLLGDPFRPLPQQVNLTDGRWHRVAVSIDGEMVTLVADCEAQPPVLGHGPRFISIAGLTVLGTQDLGEKTFEGDIQELLISPDPQAAFQACERYLPDCDNLAPAATVAPQGEPETPRPRRKGKGKGRKKGRGRKGKGRKKNKEIWTSSPPPDSAENQTSTDIPKTETPAPNLPPTPTPLVVTSTVTTGLNATILERSLDPDSGTELGTLETKAAREDEEGDDSTMGPDFRAAEYPSRTQFQIFPGAGEKGAKGEPAVIEKGQQFEGPPGAPGPQGVVGPSGPPGPPGFPGDPGPPGPAGLPGIPGIDGIRGPPGTVIMMPFQFAGGSFKGPPVSFQQAQAQAVLQQTQLSMKGPPGPVGLTGRPGPVGLPGHPGLKGEEGAEGPQGPRGLQGPHGPPGRVGKMGRPGADGARGLPGDTGPKGDRGFDGLPGLPGEKGQRGDFGHVGQPGPPGEDGERGAEGPPGPTGQAGEPGPRGLLGPRGSPGPTGRPGVTGIDGAPGAKGNVGPPGEPGPPGQQGNHGSQGLPGPQGLIGTPGEKGPPGNPGIPGLPGSDGPLGHPGHEGPTGEKGAQGPPGSAGPPGYPGPRGVKGTSGNRGLQGEKGEKGEDGFPGFKGDVGLKGDQGKPGAPGPRGEDGPEGPKGQAGQAGEEGPPGSAGEKGKLGVPGLPGYPGRPGPKGSIGFPGPLGPIGEKGKSGKTGQPGLEGERGPPGSRGERGQPGATGQPGPKGDVGQDGAPGIPGEKGLPGLQGPPGFPGPKGPPGHQGKDGRPGHPGQRGELGFQGQTGPPGPAGVLGPQGKTGEVGPLGERGPPGPPGPPGEQGLPGLEGREGAKGELGPPGPLGKEGPAGLRGFPGPKGGPGDPGPTGLKGDKGPPGPVGANGSPGERGPLGPAGGIGLPGQSGSEGPVGPAGKKGSRGERGPPGPTGKDGIPGPLGPLGPPGAAGPSGEEGDKGDVGAPGHKGSKGDKGDAGPPGQPGIRGPAGHPGPPGADGAQGRRGPPGLFGQKGDDGVRGFVGVIGPPGLQGLPGPPGEKGEVGDVGSMGPHGAPGPRGPQGPTGSEGTPGLPGGVGQPGAVGEKGERGDAGDPGPPGAPGIPGPKGDIGEKGDSGPSGAAGPPGKKGPPGEDGAKGSVGPTGLPGDLGPPGDPGVSGIDGSPGEKGDPGDVGGPGPPGASGEPGAPGPPGKRGPSGHMGREGREGEKGAKGEPGPDGPPGRTGPMGARGPPGRVGPEGLRGIPGPVGEPGLLGAPGQMGPPGPLGPSGLPGLKGDTGPKGEKGHIGLIGLIGPPGEAGEKGDQGLPGVQGPPGPKGDPGPPGPIGSLGHPGPPGVAGPLGQKGSKGSPGSMGPRGDTGPAGPPGPPGAPAELHGLRRRRRFVPVPLPVVEGGLEEVLASLTSLSLELEQLRRPPGTAERPGLVCHELHRNHPHLPDGEYWIDPNQGCARDSFRVFCNFTAGGETCLYPDKKFEIVKLASWSKEKPGGWYSTFRRGKKFSYVDADGSPVNVVQLNFLKLLSATARQNFTYSCQNAAAWLDEATGDYSHSARFLGTNGEELSFNQTTAATVSVPQDGCRLRKGQTKTLFEFSSSRAGFLPLWDVAATDFGQTNQKFGFELGPVCFSS

SEQ ID NO: 17 - DNA, 2704 bp, Homo sapiens
ccgaacaggattctttcacccaggcatctcctccagagggatccgccagcccgtccagcagcaccatgtgggtgaccaaactcctgccagccctgctgctgcagcatgtcctcctgcatctcctcctgctccccatcgccatcccctatgcagagggacaaaggaaaagaagaaatacaattcatgaattcaaaaaatcagcaaagactaccctaatcaaaatagatccagcactgaagataaaaaccaaaaaagtgaatactgcagaccaatgtgctaatagatgtactaggaataaaggacttccattcacttgcaaggcttttgtttttgataaagcaagaaaacaatgcctctggttccccttcaatagcatgtcaagtggagtgaaaaaagaatttggccatgaatttgacctctatgaaaacaaagactacattagaaactgcatcattggtaaaggacgcagctacaagggaacagtatctatcactaagagtggcatcaaatgtcagccctggagttccatgataccacacgaacacagctttttgccttcgagctatcggggtaaagacctacaggaaaactactgtcgaaatcctcgaggggaagaagggggaccctggtgtttcacaagcaatccagaggtacgctacgaagtctgtgacattcctcagtgttcagaagttgaatgcatgacctgcaatggggagagttatcgaggtctcatggatcatacagaatcaggcaagatttgtcagcgctgggatcatcagacaccacaccggcacaaattcttgcctgaaagatatcccgacaagggctttgatgataattattgccgcaatcccgatggccagccgaggccatggtgctatactcttgaccctcacacccgctgggagtactgtgcaattaaaacatgcgctgacaatactatgaatgacactgatgttcctttggaaacaactgaatgcatccaaggtcaaggagaaggctacaggggcactgtcaataccatttggaatggaattccatgtcagcgttgggattctcagtatcctcacgagcatgacatgactcctgaaaatttcaagtgcaaggacctacgagaaaattactgccgaaatccagatgggtctgaatcaccctggtgttttaccactgatccaaacatccgagttggctactgctcccaaattccaaactgtgatatgtcacatggacaagattgttatcgtgggaatggcaaaaattatatgggcaacttatcccaaacaagatctggactaacatgttcaatgtgggacaagaacatggaagacttacatcgtcatatcttctgggaaccagatgcaagtaagctgaatgagaattactgccgaaatccagatgatgatgctcatggaccctggtgctacacgggaaatccactcattccttgggattattgccctatttctcgttgtgaaggtgataccacacctacaatagtcaatttagaccatcccgtaatatcttgtgccaaaacgaaacaattgcgagttgtaaatgggattccaacacgaacaaacataggatggatggttagtttgagatacagaaataaacatatctgcggaggatcattgataaaggagagttgggttcttactgcacgacagtgtttcccttctcgagacttgaaagattatgaagcttggcttggaattcatgatgtccacggaagaggagatgagaaatgcaaacaggttctcaatgtttcccagctggtatatggccctgaaggatcagatctggttttaatgaagcttgccaggcctgctgtcctggatgattttgttagtacgattgatttacctaattatggatgcacaattcctgaaaagaccagttgcagtgtttatggctggggctacactggattgatcaactatgatggcctattacgagtggcacatctctatataatgggaaatgagaaatgcagccagcatcatcgagggaaggtgactctgaatgagtctgaaatatgtgctggggctgaaaagattggatcaggaccatgtgagggggattatggtggcccacttgtttgtgagcaacataaaatgagaatggttcttggtgtcattgttcctggtcgtggatgtgccattccaaatcgtcctggtatttttgtccgagtagcatattatgcaaaatggatacacaaaattattttaacatataaggtaccacagtcatagctgaagtaagtgtgtctgaagcacccaccaatacaactgtcttttacatgaagatttcagagaatgtggaatttaaaatgtcacttacaacaatcctaagacaactactggagagtcatgtttgttgaaattctcattaatgtttatgggtgttttctgttgttttgtttgtcagtgttattttgtcaatgttgaagtgaattaaggtacatgcaagtgtaataacatatctcctgaagatacttgaatggattaaaaaaacacacaggtatatttgctggatgataaagatttcatgggaaaaaaaatcaattaatctgtctaagctgctttctgatgttggtttcttaataatgagtaaaccacaaattaaatgttattttaacctcaccaaaacaatttataccttgtgtccctaaattgtagccctatattaaattatattacatttc

SEQ ID NO: 18 - protein, 728 aa, Homo sapiens
MWVTKLLPALLLQHVLLHLLLLPIAIPYAEGQRKRRNTIHEFKKSAKTTLIKIDPALKIKTKKVNTADQCANRCTRNKGLPFTCKAFVFDKARKQCLWFPFNSMSSGVKKEFGHEFDLYENKDYIRNCIIGKGRSYKGTVSITKSGIKCQPWSSMIPHEHSFLPSSYRGKDLQENYCRNPRGEEGGPWCFTSNPEVRYEVCDIPQCSEVECMTCNGESYRGLMDHTESGKICQRWDHQTPHRHKFLPERYPDKGFDDNYCRNPDGQPRPWCYTLDPHTRWEYCAIKTCADNTMNDTDVPLETTECIQGQGEGYRGTVNTIWNGIPCQRWDSQYPHEHDMTPENFKCKDLRENYCRNPDGSESPWCFTTDPNIRVGYCSQIPNCDMSHGQDCYRGNGKNYMGNLSQTRSGLTCSMWDKNMEDLHRHIFWEPDASKLNENYCRNPDDDAHGPWCYTGNPLIPWDYCPISRCEGDTTPTIVNLDHPVISCAKTKQLRVVNGIPTRTNIGWMVSLRYRNKHICGGSLIKESWVLTARQCFPSRDLKDYEAWLGIHDVHGRGDEKCKQVLNVSQLVYGPEGSDLVLMKLARPAVLDDFVSTIDLPNYGCTIPEKTSCSVYGWGYTGLINYDGLLRVAHLYIMGNEKCSQHHRGKVTLNESEICAGAEKIGSGPCEGDYGGPLVCEQHKMRMVLGVIVPGRGCAIPNRPGIFVRVAYYAKWIHKIILTYKVPQS

SEQ ID NO: 19 - DNA, 6194 bp, Homo sapiens
actaactcgcggctgcaggatcagcgtctggaagcagacgtttcggctacagacccagagaggaggagctggagatcaggaggcgtgagccgccaagagtttgcagaatctgtggtgtgaatgaactgggggcacctgggcgcacagatcgccccccttcccccgccccgggccacagttgagtagtggtacatttttttcaccctcttgtgaagaatttctttttattattatttgtcgtaaggtcttttgcacaatcacgcccacatttggggttggaaagccctaattaccgccgtcgctgatggacgttggaaacggagcgcctctccgtggaacagttgcctgcgcgccctcgccggaccggcggctccctagttgcgccccgaccaggccctgcccttgctgccggctcgcgcgcgtccgcgccccctccattcctgggcgcatcccagctctgccccaactcgggagtccaggcccgggcgccagtgcccgcttcagctccggttcactgcgcccgccggacgcgcgccggaggactccgcagccctgctcctgaccgtccccccaggcttaacccggtcgctccgctcggattcctcggctgcgctcgctcgggtggcgacttcctccccgcgccccctccccctcgccatgaagaagtccattggaatattaagcccaggagttgctttggggatggctggaagtgcaatgtcttccaagttcttcctagtggctttggccatatttttctccttcgcccaggttgtaattgaagccaattcttggtggtcgctaggtatgaataaccctgttcagatgtcagaagtatatattataggagcacagcctctctgcagccaactggcaggactttctcaaggacagaagaaactgtgccacttgtatcaggaccacatgcagtacatcggagaaggcgcgaagacaggcatcaaagaatgccagtatcaattccgacatcgaaggtggaactgcagcactgtggataacacctctgtttttggcagggtgatgcagataggcagccgcgagacggccttcacatacgcggtgagcgcagcaggggtggtgaacgccatgagccgggcgtgccgcgagggcgagctgtccacctgcggctgcagccgcgccgcgcgccccaaggacctgccgcgggactggctctggggcggctgcggcgacaacatcgactatggctaccgctttgccaaggagttcgtggacgcccgcgagcgggagcgcatccacgccaagggctcctacgagagtgctcgcatcctcatgaacctgcacaacaacgaggccggccgcaggacggtgtacaacctggctgatgtggcctgcaagtgccatggggtgtccggctcatgtagcctgaagacatgctggctgcagctggcagacttccgcaaggtgggtgatgccctgaaggagaagtacgacagcgcggcggccatgcggctcaacagccggggcaagttggtacaggtcaacagccgcttcaactcgcccaccacacaagacctggtctacatcgaccccagccctgactactgcgtgcgcaatgagagcaccggctcgctgggcacgcagggccgcctgtgcaacaagacgtcggagggcatggatggctgcgagctcatgtgctgcggccgtggctacgaccagttcaagaccgtgcagacggagcgctgccactgcaagttccactggtgctgctacgtcaagtgcaagaagtgcacggagatcgtggaccagtttgtgtgcaagtagtgggtgccacccagcactcagccccgctcccaggacccgcttatttatagaaagtacagtgattctggtttttggtttttagaaatattttttatttttccccaagaattgcaaccggaaccattttttttcctgttaccatctaagaactctgtggtttattattaatattataattattatttggcaataatgggggtgggaaccaagaaaaatatttattttgtggatctttgaaaaggtaatacaagacttcttttgatagtatagaatgaaggggaaataacacataccctaacttagctgtgtggacatggtacacatccagaaggtaaagaaatacattttctttttctcaaatatgccatcatatgggatgggtaggttccagttgaaagagggtggtagaaatctattcacaattcagcttctatgaccaaaatgagttgtaaattctctggtgcaagataaaaggtcttgggaaaacaaaacaaaacaaaacaaacctcccttccccagcagggctgctagcttgctttctgcattttcaaaatgataatttacaatggaaggacaagaatgtcatattctcaaggaaaaaaggtatatcacatgtctcattctcctcaaatattccatttgcagacagaccgtcatattctaatagctcatgaaatttgggcagcagggaggaaagtccccagaaattaaaaaatttaaaactcttatgtcaagatgttgatttgaagctgttataagaattaggattccagattgtaaaaagatccccaaatgattctggacactagatttttttgtttggggaggttggcttgaacataaatgaaaatatcctgttattttcttagggatacttggttagtaaattataatagtaaaaataatacatgaatcccattcacaggttctcagcccaagcaacaaggtaattgcgtgccattcagcactgcaccagagcagacaacctatttgaggaaaaacagtgaaatccaccttcctcttcacactgagccctctctgattcctccgtgttgtgatgtgatgctggccacgtttccaaacggcagctccactgggtcccctttggttgtaggacaggaaatgaaacattaggagctctgcttggaaaacagttcactacttagggatttttgtttcctaaaacttttattttgaggagcagtagttttctatgttttaatgacagaacttggctaatggaattcacagaggtgttgcagcgtatcactgttatgatcctgtgtttagattatccactcatgcttctcctattgtactgcaggtgtaccttaaaactgttcccagtgtacttgaacagttgcatttataaggggggaaatgtggtttaatggtgcctgatatctcaaagtcttttgtacataacatatatatatatatacatatatataaatataaatataaatatatctcattgcagccagtgatttagatttacagtttactctggggttatttctctgtctagagcattgttgtccttcactgcagtccagttgggattattccaaaagttttttgagtcttgagcttgggctgtggccctgctgtgatcataccttgagcacgacgaagcaaccttgtttctgaggaagcttgagttctgactcactgaaatgcgtgttgggttgaagatatcttttttcttttctgcctcacccctttgtctccaacctccatttctgttcactttgtggagagggcattacttgttcgttatagacatggacgttaagagatattcaaaactcagaagcatcagcaatgtttctcttttcttagttcattctgcagaatggaaacccatgcctattagaaatgacagtacttattaattgagtccctaaggaatattcagcccactacatagatagcttttttttttttttttttaataaggacacctctttccaaacagtgccatcaaatatgttcttatctcagacttacgttgttttaaaagtttggaaagatacacatctttcataccccccttaggcaggttggctttcatatcacctcagccaactgtggctcttaatttattgcataatgatattcacatcccctcagttgcagtgaattgtgagcaaaagatcttgaaagcaaaaagcactaattagtttaaaatgtcacttttttggtttttattatacaaaaaccatgaagtactttttttatttgctaaatcagattgttcctttttagtgactcatgtttatgaagagagttgagtttaacaatcctagcttttaaaagaaactatttaatgtaaaatattctacatgtcattcagatattatgtatatcttctagcctttattctgtacttttaatgtacatatttctgtcttgcgtgatttgtatatttcactggtttaaaaaacaaacatcgaaaggcttatgccaaatggaagatagaatataaaataaaacgttacttgtatattggtaagtggtttcaattgtccttcagataattcatgtggagatttttggagaaaccatgacggatagtttaggatgactacatgtcaaagtaataaaagagtggtgaattttaccaaaaccaagctatttggaagcttcaaaaggtttctatatgtaatggaacaaaaggggaattctcttttcctatatatgttccttacaaaaaaaaaaaaaaaagaaatcaagcagatggcttaaagctggttataggattgctcacattcttttagcattatgcatgtaacttaattgttttagagcgtgttgctgttgtaacatcccagagaagaatgaaaaggcacatgcttttatccgtgaccagatttttagtccaaaaaaatgtatttttttgtgtgtttaccactgcaactattgcacctctctatttgaatttactgtggaccatgtgtggtgtctctatgccctttgaaagcagtttttataaaaagaaagcccgggtctgcagagaatgaaaactggttggaaactaaaggttcattgtgttaagtgcaattaatacaagttattgtgcttttcaaaaatgtacacggaaatctggacagtgctccacagattgatacattagcctttgctttttctctttccggataaccttgtaacatattgaaaccttttaaggatgccaagaatgcattattccacaaaaaaacagcagaccaacatatagagtgtttaaaatagcatttctgggcaaattcaaactcttgtggttctaggactcacatctgtttcagtttttcctcagttgtatattgaccagtgttctttattgcaaaaacatatacccgatttagcagtgtcagcgtattttttcttctcatcctggagcgtattcaagatcttcccaatacaagaaaattaataaaaaatttatatataggcagcagcaaaagagccatgttcaaaatagtcattatgggctcaaatagaaagaagacttttaagttttaatccagtttatctgttgagttctgtgagctactgacctcctgagactggcactgtgtaagttttagttgcctaccctagctcttttctcgtacaattttgccaataccaagtttcaatttgtttttacaaaacattattcaagccactagaattatcaaatatgacgctatagcagagtaaatactctgaataagagaccggtactagctaactccaagagatcgttagcagcatcagtccacaaacacttagtggcccacaatatatagagagatagaaaaggtagttataacttgaagcatgtatttaatgcaaataggcacgaaggcacaggtctaaaatactacattgtcactgtaagctatacttttaaaatatttattttttttaaagtattttctagtcttttctctctctgtggaatggtgaaagagagatgccgtgttttgaaagtaagatgatgaaatgaatttttaattcaagaaacattcagaaacataggaattaaaacttagagaaatgatctaatttccctgttcacacaaactttacactttaatctgatgattggatattttattttagtgaaacatcatcttgttagctaactttaaaaaatggatgtagaatgattaaaggttggtatgatttttttttaatgtatcagtttgaacctagaatattgaattaaaatgctgtctcagtattttaaaagcaaaaaaggaatggaggaaaattgcatcttagaccatttttatatgcagtgtacaatttgctgggctagaaatgagataaagattatttatttttgttcatatcttgtacttttctattaaaatcattttatgaaatccaaaaaaaaaaaaaaaaaa

SEQ ID NO: 20 - protein, 380 aa, Homo sapiens
MKKSIGILSPGVALGMAGSAMSSKFFLVALAIFFSFAQVVIEANSWWSLGMNNPVQMSEVYIIGAQPLCSQLAGLSQGQKKLCHLYQDHMQYIGEGAKTGIKECQYQFRHRRWNCSTVDNTSVFGRVMQIGSRETAFTYAVSAAGVVNAMSRACREGELSTCGCSRAARPKDLPRDWLWGGCGDNIDYGYRFAKEFVDARERERIHAKGSYESARILMNLHNNEAGRRTVYNLADVACKCHGVSGSCSLKTCWLQLADFRKVGDALKEKYDSAAAMRLNSRGKLVQVNSRFNSPTTQDLVYIDPSPDYCVRNESTGSLGTQGRLCNKTSEGMDGCELMCCGRGYDQFKTVQTERCHCKFHWCCYVKCKKCTEIVDQFVCK

SEQ ID NO: 21 - DNA, 760 bp, Homo sapiens
gaggaaccgagaggctgagactaacccagaaacatccaattctcaaactgaagctcgcactctcgcctccagcatgaaagtctctgccgcccttctgtgcctgctgctcatagcagccaccttcattccccaagggctcgctcagccagatgcaatcaatgccccagtcacctgctgttataacttcaccaataggaagatctcagtgcagaggctcgcgagctatagaagaatcaccagcagcaagtgtcccaaagaagctgtgatcttcaagaccattgtggccaaggagatctgtgctgaccccaagcagaagtgggttcaggattccatggaccacctggacaagcaaacccaaactccgaagacttgaacactcactccacaacccaagaatctgcagctaacttattttcccctagctttccccagacaccctgttttattttattataatgaattttgtttgttgatgtgaaacattatgccttaagtaatgttaattcttatttaagttattgatgttttaagtttatctttcatggtactagtgttttttagatacagagacttggggaaattgcttttcctcttgaaccacagttctacccctgggatgttttgagggtctttgcaagaatcattaatacaaagaattttttttaacattccaatgcattgctaaaatattattgtggaaatgaatattttgtaactattacaccaaataaatatatttttgtacaaaaaaaaaaaaaaa

SEQ ID NO: 22 - protein, 99 aa, Homo sapiens
MKVSAALLCLLLIAATFIPQGLAQPDAINAPVTCCYNFTNRKISVQRLASYRRITSSKCPKEAVIFKTIVAKEICADPKQKWVQDSMDHLDKQTQTPKT

SEQ ID NO: 23 - DNA, 800 bp, Homo sapiens
acacagagagaaaggctaaagttctctggaggatgtggctgcagagcctgctgctcttgggcactgtggcctgcagcatctctgcacccgcccgctcgcccagccccagcacgcagccctgggagcatgtgaatgccatccaggaggcccggcgtctcctgaacctgagtagagacactgctgctgagatgaatgaaacagtagaagtcatctcagaaatgtttgacctccaggagccgacctgcctacagacccgcctggagctgtacaagcagggcctgcggggcagcctcaccaagctcaagggccccttgaccatgatggccagccactacaagcagcactgccctccaaccccggaaacttcctgtgcaacccagattatcacctttgaaagtttcaaagagaacctgaaggactttctgcttgtcatcccctttgactgctgggagccagtccaggagtgagaccggccagatgaggctggccaagccggggagctgctctctcatgaaacaagagctagaaactcaggatggtcatcttggagggaccaaggggtgggccacagccatggtgggagtggcctggacctgccctgggccacactgaccctgatacaggcatggcagaagaatgggaatattttatactgacagaaatcagtaatatttatatatttatatttttaaaatatttatttatttatttatttaagttcatattccatatttattcaagatgttttaccgtaataattattattaaaaatatgcttctacttgaaaaaaaaaaaaaaa

SEQ ID NO: 24 - protein, 144 aa, Homo sapiens
MWLQSLLLLGTVACSISAPARSPSPSTQPWEHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQE

SEQ ID NO: 25 - DNA, 2358 bp, Homo sapiens
aaactcacacaacaactcttccccgctgagaggagacagccagtgcgactccaccctccagctcgacggcagccgccccggccgacagccccgagacgacagcccggcgcgtcccggtccccacctccgaccaccgccagcgctccaggccccgccgctccccgctcgccgccaccgcgccctccgctccgcccgcagtgccaaccatgaccgccgccagtatgggccccgtccgcgtcgccttcgtggtcctcctcgccctctgcagccggccggccgtcggccagaactgcagcgggccgtgccggtgcccggacgagccggcgccgcgctgcccggcgggcgtgagcctcgtgctggacggctgcggctgctgccgcgtctgcgccaagcagctgggcgagctgtgcaccgagcgcgacccctgcgacccgcacaagggcctcttctgtgacttcggctccccggccaaccgcaagatcggcgtgtgcaccgccaaagatggtgctccctgcatcttcggtggtacggtgtaccgcagcggagagtccttccagagcagctgcaagtaccagtgcacgtgcctggacggggcggtgggctgcatgcccctgtgcagcatggacgttcgtctgcccagccctgactgccccttcccgaggagggtcaagctgcccgggaaatgctgcgaggagtgggtgtgtgacgagcccaaggaccaaaccgtggttgggcctgccctcgcggcttaccgactggaagacacgtttggcccagacccaactatgattagagccaactgcctggtccagaccacagagtggagcgcctgttccaagacctgtgggatgggcatctccacccgggttaccaatgacaacgcctcctgcaggctagagaagcagagccgcctgtgcatggtcaggccttgcgaagctgacctggaagagaacattaagaagggcaaaaagtgcatccgtactcccaaaatctccaagcctatcaagtttgagctttctggctgcaccagcatgaagacataccgagctaaattctgtggagtatgtaccgacggccgatgctgcaccccccacagaaccaccaccctgccggtggagttcaagtgccctgacggcgaggtcatgaagaagaacatgatgttcatcaagacctgtgcctgccattacaactgtcccggagacaatgacatctttgaatcgctgtactacaggaagatgtacggagacatggcatgaagccagagagtgagagacattaactcattagactggaacttgaactgattcacatctcatttttccgtaaaaatgatttcagtagcacaagttatttaaatctgtttttctaactgggggaaaagattcccacccaattcaaaacattgtgccatgtcaaacaaatagtctatcaaccccagacactggtttgaagaatgttaagacttgacagtggaactacattagtacacagcaccagaatgtatattaaggtgtggctttaggagcagtgggagggtaccagcagaaaggttagtatcatcagatagcatcttatacgagtaatatgcctgctatttgaagtgtaattgagaaggaaaattttagcgtgctcactgacctgcctgtagccccagtgacagctaggatgtgcattctccagccatcaagagactgagtcaagttgttccttaagtcagaacagcagactcagctctgacattctgattcgaatgacactgttcaggaatcggaatcctgtcgattagactggacagcttgtggcaagtgaatttgcctgtaacaagccagattttttaaaatttatattgtaaatattgtgtgtgtgtgtgtgtgtgtatatatatatatatgtacagttatctaagttaatttaaagttgtttgtgcctttttatttttgtttttaatgctttgatatttcaatgttagcctcaatttctgaacaccataggtagaatgtaaagcttgtctgatcgttcaaagcatgaaatggatacttatatggaaattctgctcagatagaatgacagtccgtcaaaacagattgtttgcaaaggggaggcatcagtgtccttggcaggctgatttctaggtaggaaatgtggtagcctcacttttaatgaacaaatggcctttattaaaaactgagtgactctatatagctgatcagttttttcacctggaagcatttgtttctactttgatatgactgtttttcggacagtttatttgttgagagtgtgaccaaaagttacatgtttgcacctttctagttgaaaataaagtgtatattttttctataaaaaaaaaaaaaaaaa

SEQ ID NO: 26 - protein, 349 aa, Homo sapiens
MTAASMGPVRVAFVVLLALCSRPAVGQNCSGPCRCPDEPAPRCPAGVSLVLDGCGCCRVCAKQLGELCTERDPCDPHKGLFCDFGSPANRKIGVCTAKDGAPCIFGGTVYRSGESFQSSCKYQCTCLDGAVGCMPLCSMDVRLPSPDCPFPRRVKLPGKCCEEWVCDEPKDQTVVGPALAAYRLEDTFGPDPTMIRANCLVQTTEWSACSKTCGMGISTRVTNDNASCRLEKQSRLCMVRPCEADLEENIKKGKKCIRTPKISKPIKFELSGCTSMKTYRAKFCGVCTDGRCCTPHRTTTLPVEFKCPDGEVMKKNMMFIKTCACHYNCPGDNDIFESLYYRKMYGDMA

SEQ ID NO: 27 - DNA, 1574 bp, Homo sapiens
tcaccacggcggcagccctttaaacccctcacccagccagcgccccatcctgtctgtccgaacccagacacaagtcttcactccttcctgcgagccctgaggaagccttgtgagtgcattggctggggcttggagggaagttgggctggagctggacaggagcagtgggtgcatttcaggcaggctctcctgaggtcccaggcgccagctccagctccctggctagggaaacccaccctctcagtcagcatgggggcccaagctccaggcagggtgggctggatcactagcgtcctggatctctctcagactgggcagccccgggctcattgaaatgccccggatgacttggctagtgcagaggaattgatggaaaccaccggggtgagagggaggctccccatctcagccagccacatccacaaggtgtgtgtaagggtgcaggcgccggccggttaggccaaggctctactgtctgttgcccctccaggagaacttccaaggagctttccccagacatggccaacaagggtccttcctatggcatgagccgcgaagtgcagtccaaaatcgagaagaagtatgacgaggagctggaggagcggctggtggagtggatcatagtgcagtgtggccctgatgtgggccgcccagaccgtgggcgcttgggcttccaggtctggctgaagaatggcgtgattctgagcaagctggtgaacagcctgtaccctgatggctccaagccggtgaaggtgcccgagaacccaccctccatggtcttcaagcagatggagcaggtggctcagttcctgaaggcggctgaggactatggggtcatcaagactgacatgttccagactgttgacctctttgaaggcaaagacatggcagcagtgcagaggaccctgatggctttgggcagcttggcagtgaccaagaatgatgggcactaccgtggagatcccaactggtttatgaagaaagcgcaggagcataagagggaattcacagagagccagctgcaggagggaaagcatgtcattggccttcagatgggcagcaacagaggggcctcccaggccggcatgacaggctacggacgacctcggcagatcatcagttagagcggagagggctagccctgagcccggccctcccccagctccttggctgcagccatcccgcttagcctgcctcacccacacccgtgtggtaccttcagccctggccaagctttgaggctctgtcactgagcaatggtaactgcacctgggcagctcctccctgtgcccccagcctcagcccaacttcttacccgaaagcatcactgccttggcccctccctcccggctgcccccatcacctctactgtctcctccctgggctaagcaggggagaagcgggctgggggtagcctggatgtgggccaagtccactgtcctccttggcggcaaaagcccattgaagaagaaccagcccagcctgccccctatcttgtcctggaatatttttggggttggaactcaaaaaaaaaaaaaaaaaatcaatcttttctcaaaaaaaaaaaaaaaaaa

SEQ ID NO: 28 - protein, 199 aa, Homo sapiens
MANKGPSYGMSREVQSKIEKKYDEELEERLVEWIIVQCGPDVGRPDRGRLGFQVWLKNGVILSKLVNSLYPDGSKPVKVPENPPSMVFKQMEQVAQFLKAAEDYGVIKTDMFQTVDLFEGKDMAAVQRTLMALGSLAVTKNDGHYRGDPNWFMKKAQEHKREFTESQLQEGKHVIGLQMGRGASQAGMTGYGRPRQIIS

(19) United States
(12) Patent Application Publication
(10) Pub. No.: US 2023/0092052 A1
(43) Pub. Date: Mar. 23, 2023

(54) INTERPENETRATING NETWORK HYDROGELS WITH INDEPENDENTLY TUNABLE STIFFNESS

(71) Applicant: President and Fellows of Harvard College, Cambridge, MA (US)

(72) Inventors: Cristiana Branco da Cunha, Cambridge, MA (US); Ovijit Chaudhuri, San Mateo, CA (US); David J. Mooney, Sudbury, MA (US)

(21) Appl. No.: 17/748,330

(22) Filed: May 19, 2022

Related U.S. Application Data
(63) Continuation of application No. 15/313,316, filed on Nov. 22, 2016, now abandoned.
(60) Provisional application No. 62/011,517, filed on Jun. 12, 2014.

(51) Int. Cl. A61L 26/00; A61L 15/42; A61L 15/22; A61L 15/44; A61L 15/60; A61L 15/46

(52) U.S. Cl. CPC A61L 26/0052; A61L 26/0066; A61L 26/008; A61L 26/0085; A61L 15/425; A61L 15/225; A61L 15/44; A61L 15/60; A61L 15/46; A61L 26/0057; A61L 2300/64; A61L 2300/414; A61L 2400/12; A61L 2300/41; A61L 2300/406

(57) ABSTRACT

Interpenetrating network hydrogels with independently tunable stiffness enhance tissue regeneration and wound healing.

### RELATED APPLICATIONS

This application is a continuation of U.S. application Ser. No. 15/313,316, filed on Nov. 22, 2016; which is a 35 U.S.C. § 371 national stage filing of International Application No. PCT/US2015/035580, filed on Jun. 12, 2015; which claims priority to U.S. Provisional Application No. 62/011,517, filed on Jun. 12, 2014. The entire contents of each of the foregoing applications are hereby incorporated herein by reference.

### FIELD OF THE INVENTION

The present invention relates to hydrogels for tissue regeneration and wound healing.

### SEQUENCE LISTING

The instant application contains a Sequence Listing which has been submitted in ASCII format via EFS-Web and is hereby incorporated by reference in its entirety. Said ASCII copy, created on May 18, 2022, is named 117820-09403_SL.txt and is 153,324 bytes in size.

### BACKGROUND OF THE INVENTION

Wound healing is a complex physiological process orchestrated by multiple cell types, soluble factors and extracellular matrix components. Many cutaneous injuries heal rapidly within a week or two, though often leading to the formation of a mass of fibrotic tissue which is neither aesthetical nor functional. However, several pathogenic abnormalities, ranging from diabetic ulcers to infection or continued trauma, contribute to failure to heal. Chronic nonhealing wounds are a cause of significant morbidity and mortality, and constitute a huge burden in public health care with estimated costs of more than $3 billion per year. The goal of wound care therapies is to regenerate tissues such that the structural and functional properties are restored to the levels before injury.

The wound dressing market is expanding rapidly and is estimated to be valued at $21.6 billion by 2018. Wound dressing materials have been engineered to aid and enhance healing once they are deposited on the wounds. In the current wound dressing market, no single dressing is suitable for all wounds. Wound healing biomaterials are increasingly being designed to incorporate bioactive molecules to promote healing. Current developments in the field include more sophisticated wound dressing materials that often incorporate antimicrobial, antibacterial, and anti-inflammatory agents. However, the importance of mechanical forces in the context of wound dressing design, e.g., the impact of the wound dressing physical properties on the biology of cells orchestrating wound healing, has been often overlooked. For example, there is a lack of wound healing materials that mimic the stiffness and physiological environment of natural tissues at the wound site. There is also a need for wound healing biomaterials that are cost-effectively manufactured and easily customizable depending on the type of injury/wound, without the need for exogenous cytokines, growth factors, or bioactive drugs.

### SUMMARY OF THE INVENTION

The invention addresses these needs and features a universal platform—a hydrogel material—useful for aiding the healing process of a tissue. The hydrogel contains collagen, which provides sites for cell attachment and mimics the natural physiological environment of a cell. Moreover, the invention provides a clean way to tune the stiffness of the hydrogel independently of other mechanical/structural variables. As such, the hydrogel is customizable to mimic the natural stiffness of the tissue at a target site, e.g., at a site that requires healing. For example, the stiffness of the hydrogel is tuned specifically to match that of a normal, healthy tissue.

Accordingly, this invention provides a composition and method to aid and enhance wound healing, e.g., for the treatment of chronic non-healing wounds. Diabetic ulcers, ischemia, infection, and continued trauma, contribute to the failure to heal and demand sophisticated wound care therapies. Hydrogels comprising interpenetrating networks (IPNs) of collagen (e.g., collagen-I) and alginate permit the control of cell behavior, e.g., dermal fibroblast behavior, simply by tuning or altering the storage moduli of the hydrogel, e.g., in a dermal dressing material. The storage modulus of a material, such as a hydrogel, is a measure of the stored energy, which represents the elastic portion of a viscoelastic material. In accordance with the methods of the invention, fully interpenetrating networks of collagen and alginate were fabricated in which gel stiffness was tuned independently of scaffold architecture, polymer concentration or adhesion ligand density. Different storage moduli promoted dramatically different morphologies of encapsulated dermal fibroblasts, and enhanced stiffness resulted in up-regulation of key-mediators of inflammation including interleukin 10 (IL10) and prostaglandin-endoperoxide synthase 2 (PTGS2) also known as COX2. The findings presented herein show that simply modulating the storage modulus of a cutaneous dressing biomaterial deposited at a wound site, without the addition of any soluble factors, augments the progression of wound healing.

The invention provides a 3-dimensional hydrogel comprising an interpenetrating network of alginate and collagen, wherein the hydrogel comprises a storage modulus of 20 Pa or greater, e.g., 20, 30, 40, 50, 60, 70, 80, 90, 100, 150, 200, 250, 300, 400, 500, 600, or 800 Pa, 1, 2, 3, 4, 5, 10, 50, 100, 500 kPa, 1, 2, 3, 4, 5, 10, 50, 100, or 500 MPa, or greater. In some cases, the storage modulus is between 50 kPa and 50 MPa. In some examples, the storage modulus is between 30 Pa and 1200 Pa For example, the storage modulus is between 30 Pa and 400 Pa, (e.g., 400, 300, 250, 200, 150, 100, 75, 60, 55, 50, 45, 40, 35, or 30 Pa) or between 30 Pa and 300 Pa.

For example, the collagen comprises fibrillar collagen, e.g., collagen type I, II, III, V, XI, XXIV, or XXVII. Other types of collagen are also included in the invention. In one embodiment, the collagen comprises type I collagen, also called collagen-I.

In some cases, the alginate does not contain any molecules to which cells adhere. For example, the alginate is not modified by a cell adhesion molecule, i.e., the alginate lacks a cell adhesion molecule, e.g., a polypeptide comprising the amino acid sequence, arginine-glycine-aspartate (RGD).

In the hydrogel, alginate is crosslinked to form a mesh structure. The hydrogels of the invention do not comprise any covalent crosslinks. In particular, the alginate is not covalently cross-linked. The alginate is non-covalently or ionically cross-linked. In some embodiments, the alginate is ionically crosslinked, e.g., by divalent or trivalent cations. Exemplary divalent cations include Ca²⁺, Mg²⁺, Sr²⁺, Ba²⁺, and Be²⁺. Exemplary trivalent cations include Al³⁺ and Fe³⁺. In one embodiment, the divalent cation comprises Ca²⁺. For example, the alginate is crosslinked by a concentration of 2 mM-10 mM Ca²⁺, e.g., at least about 5 mM, e.g., at least about 9 mM Ca²⁺.

In some examples, the alginate comprises a molecular weight of at least about 30 kDa, e.g., at least about 30, 40, 50, 60, 70, 80, 90, 100, 120, 140, 160, 180, 190, 200, 210, 220, 230, 240, 250, 260, 270, 280, 290, 300 kDa, or greater. For example, the molecular weight of the alginate is at least about 100 kDa, e.g., at least about 100, 120, 140, 160, 180, 190, 200, 210, 220, 230, 240, 250, 260, 270, 280, 290, 300 kDa, or greater. For example, the molecular weight of the alginate is about 200 kDa, 250 kDa, or 280 kDa.

In some embodiments, the hydrogel comprises multidirectional collagen fibrils (e.g., collagen-I fibrils), e.g., the hydrogel comprises collagen (e.g., collagen-I) fibrils that are not aligned/parallel. For example, the alginate mesh is intercalated by the collagen (e.g., collagen-I) fibrils. In other words, the collagen-I fibril(s) are reversibly included/inserted within the alginate mesh or are layered together with the alginate mesh. In some examples, the collagen protein comprises full length collagen subunits. In other examples, the collagen protein comprises fragments of collagen subunits, e.g., containing less than 100% of the amino acid length of a full length subunit polypeptide (e.g., less than 100, 99, 98, 97, 96, 95, 90, 85, 80, 75, 70, 65, 60, 55, 50, 40, 30, 20, or 10%).

In some cases, the hydrogel comprises a collagen (e.g., collagen-I) concentration of about 1.5 mg/mL, e.g., 1-2 mg/mL. In some examples, the hydrogel comprises an alginate concentration of about 5 mg/mL, e.g., 2-10 mg/mL. For example, the weight ratio of alginate to collagen in the hydrogel is about 2.5-5 (e.g., about 2.5, 3, 3.3, 3.5, 4, 4.5, or 5).

In some embodiments, the hydrogel comprises interconnected pores, e.g., comprising nanopores. For example, the hydrogel contains nanopores, micropores, macropores, or a combination thereof. The size of the pores permits cell migration or movement (e.g., fibroblast migration into and/or egress out of the delivery vehicle) through the pores. For example, the hydrogel comprises pores that are characterized by a diameter of 20-500 μm (e.g., 50-500 μm, or 20-300 μm). In other examples, the hydrogel comprises nanopores, e.g., pores with a diameter of about 10 nm to 20 μm. For example, the hydrogel comprises a dextran diffusion coefficient of 2.5×10⁻⁷ to 1×10⁻⁶ cm²/s.

The hydrogel of the invention comprises various relative concentrations of elements, such as carbon, oxygen, potassium, and calcium. For example, the hydrogel comprises a relative concentration of carbon of 10-50% weight/weight (e.g., 10, 20, 30, 40, or 50%), a relative concentration of oxygen of 50-70% weight/weight (e.g., 50, 55, 60, 65, or 70%), a relative concentration of potassium of 0.5-2% weight/weight (e.g., 0.5, 1, 1.5, or 2%), and/or a relative concentration of calcium of 0.5-10% weight/weight (e.g., 0.5, 1, 2, 5, 7, or 10%).

In some cases, the hydrogel further comprises a mammalian cell, such as a fibroblast. For example, the fibroblast includes a dermal fibroblast. In some examples, the cell, e.g., fibroblast, is a healthy cell (e.g., healthy fibroblast), e.g., derived/isolated from a non-injured and non-diseased tissue, such as a non-diabetic tissue. Contact of the cell with the hydrogel causes the cell to adopt or maintain an elongated or spindle-likecell shape, e.g., where the cell forms stress fiber(s). For example, contact of the cell with the hydrogel causes the cell to adopt or maintain the ability to contract and/or expand in surface area and/or volume. For example, such an ability permits the cell, e.g., fibroblast, to cover a wound and allow wound closure. In other examples, the mammalian cell comprises a stem cell, e.g., a hematopoietic stem cell, a mesenchymal stem cell, an embryonic stem cell, or an adult stem cell. For example, contact of a stem cell with the hydrogel causes the cell to adopt or maintain a spherical cell shape, e.g., where the cell does not form stress fiber(s).

In some embodiments, the mammalian cell comprises an autologous cell, allogeneic cell, or a xenogeneic cell. In some embodiments, the fibroblasts comprises an autologous fibroblast (e.g., a population of at least 10%, 20%, 30%, 40%, 50%, 60%, 70%, 80%, 90%, 95%, 98%, or more autologous fibroblasts). Alternatively or in addition, the fibroblast comprises an allogeneic or xenogeneic fibroblast. For example, the fibroblasts comprises a population of at least 10% (e.g., at least 20%, 30%, 40%, 50%, 60%, 70%, 80%, 90%, 95%, 98%, or more) allogeneic fibroblasts. For example, the fibroblast comprises a population of at least 10% (e.g., at least 20%, 30%, 40%, 50%, 60%, 70%, 80%, 90%, 95%, 98%, or more) xenogeneic fibroblasts. The fibroblasts preferably elicit a minimal adverse host response (e.g., minimal harmful inflammation and/or minimal host immune rejection of the transplanted fibroblasts).

For example, the hydrogels of the invention are used as a wound dressing materials. For example, the hydrogels of the invention are coated onto/into a wound dressing material. For example, the stiffness of the dressing materials are designed to match the stiffness of structurally intact/healthy tissue (e.g., at the site of the wound prior to injury), which can vary depending on the type of injured tissue, site of injury, natural person-to-person variations, and/or age.

The hydrogels described herein are useful for enhancing wound healing of an injured tissue, e.g., cutaneous, bony, cartilaginous, soft, vascular, or mucosal tissue.

Thus, the invention provides a wound dressing material comprising a hydrogel described herein. In some cases, the wound dressing material/hydrogel does not contain any active agents, such as anti-microbial or anti-inflammatory agents.

In other cases, the wound dressing material/hydrogel further contains a bioactive composition. Exemplary bioactive compositions include cell growth and/or cell differentiation factors. For example, a bioactive composition includes a growth factor, morphogen, differentiation factor, and/or chemoattractant. For example, the hydrogel includes vascular endothelial growth factor (VEGF), hepatocyte growth factor (HGF), or fibroblast growth factor 2 (FGF2) or a combination thereof. Other bioactive compositions include hormones, neurotransmitters, neurotransmitter or growth factor receptors, interferons, interleukins, chemokines, MMP-sensitive substrate, cytokines, colony stimulating factors and phosphatase inhibitors. Growth factors used to promote angiogenesis, wound healing, and/or tissue regeneration can be included in the hydrogel.

For example, the wound dressing materials/hydrogel further contains an anti-microbial (e.g., anti-bacterial) or anti-inflammatory agent. Exemplary anti-microbial agents include erythromycin, streptomycin, zithromycin, platensimycin, iodophor, 2% mupirocin, triple antibiotic ointment (TAO, bacitracin zinc+polymyxin B sulfate+neomycin sulfate) and others, as well as peptide anti-microbial agents. Exemplary anti-inflammatory agents include corticosteroid anti-inflammatory drugs (e.g., beclomethasone, beclometasone, budesonide, flunisolide, fluticasone propionate, triamcinolone, methylprednisolone, prednisolone, or prednisone); or non-steroidal anti-inflammatory drugs (NSAIDs) (e.g., acetylsalicylic acid, diflunisal, salsalate, choline magnesium trisalicylate, ibuprofen, dexibuprofen, naproxen, fenoprofen, ketoprofen, dexketoprofen, fluribiprofen, oxaprozin, loxoprofen, indomethacin, tolmetin, sulindac, etodolac, ketorolac, diclofenac, aceclofenac, nabumetone, piroxicam, meloxicam, tenoxicam, droxicam, lornoxicam, isoxicam, mefenamic acid, meclofenamic acid, flufenamic acid, tolfenamic acid, celecoxib, rofecoxib, valdecoxib, parecoxib, lumiracoxib, etoricoxib, firocoxib, nimesulide, licofelone, H-harpaide, or lysine clonixinate).

The invention also provides a method of promoting tissue repair, tissue regeneration, or wound healing comprising administering a hydrogel described herein to a subject in need thereof. For example, the subject contains an injured tissue, e.g., an injured cutaneous, bony, cartilaginous, soft, vascular, or mucosal tissue. In some examples, the subject has a chronic, non-healing wound, e.g., a diabetic wound or ulcer. In other embodiments, the subject has an ischemic wound, infected wound, or a wound caused by continued trauma, e.g., blunt force trauma, cuts, or scrapes.

In accordance with the methods of the invention, the hydrogel is optionally seeded with mammalian cells prior to administration, e.g., the hydrogel is encapsulated with mammalian cells prior to administration. In some cases, the mammalian cells are encapsulated within the hydrogel during the crosslinking of alginate. In other examples, the hydrogel contacts a mammalian cell after administration, e.g., the mammalian cell migrates onto and/or into the hydrogel after administration.

The hydrogels/wound dressing materials of the invention modulate the expression of various proteins in cells (e.g., fibroblasts) at or surrounding the site of administration or the site of the injured tissue. For example, the hydrogel downregulates the expression of an inflammation associated protein, e.g., IL-10 and/or COX-2, a cell adhesion or extracellular matrix protein, e.g., integrin a4 (ITGA4), metallopeptidase 1 (MMP1), or vitronectin (VTN), a collagen protein, e.g., Type IV (e.g., COL4A1 or COL4A3) or Type V (e.g., COL5A3) protein, or hepatocyte growth factor (HGF) or a member of the WNT gene family (WNTSA). For example, the expression is downregulated at the polypeptide or mRNA level. The polypeptide or mRNA level of the protein is decreased by at least 1.5-fold (e.g., at least 1.5, 2, 3, 4, 5, 6, 7, 8, 9, 10-fold, or greater) in tissues at or surrounding (e.g., within 5 cm, e.g., within 5, 4, 3, 2, 1, 0.5 cm or less of a border/perimeter of the hydrogel) the site of hydrogel administration compared to the level in the tissues prior to administration of the hydrogel.

In some embodiments, the IL-10 polypeptide or mRNA level is decreased by at least 2-fold (e.g., at least 2, 3, 4, 5, 6, 7, 8, 9, 10-fold, or greater) in tissues at or surrounding (e.g., within 5 cm, e.g., within 5, 4, 3, 2, 1, 0.5 cm or less of a border/perimeter of the hydrogel) the site of hydrogel administration compared to the level in the tissues prior to administration of the hydrogel. In some cases, the COX-2 polypeptide or mRNA level is decreased by at least 2-fold (e.g., at least 2, 3, 4, 5, 6, 7, 8, 9, 10, 12, 14, 18, 20-fold, or greater) in tissues at or surrounding (e.g., within 5 cm, e.g., within 5, 4, 3, 2, 1, 0.5 cm or less of a border/perimeter of the hydrogel) the site of hydrogel administration compared to the level in the tissues prior to administration of the hydrogel. For example, administration of the hydrogel reduces the level of inflammatory factors at a site of a wound.

In other embodiments, the hydrogel upregulates the expression of an inflammation associated protein, e.g., CCL2, colony stimulating factor 2 (CSF2), connective tissue growth factor (CTGF), and/or transgelin (TAGLN) protein. The protein is upregulated at the polypeptide or mRNA level, e.g., by at least 1.5-fold (e.g., at least 1.5, 2, 3, 4, 5, 6, 7, 8, 9, 10-fold, or greater) in tissues at or surrounding (e.g., within 5 cm, e.g., within 5, 4, 3, 2, 1, 0.5 cm or less of a border/perimeter of the hydrogel) the site of hydrogel administration compared to the level in the tissues prior to administration of the hydrogel.

For example, the subject is a mammal, e.g., a human, dog, cat, pig, cow, sheep, or horse. Preferably, the subject is a human. For example, the patient suffers from diabetes. For example, the patient suffers from a wound that is resistant to healing. In some cases, the wound is located in an extremity of the patient (e.g., an arm, leg, foot, hand, toe, or finger). For example, the patient suffers from an ulcer, e.g., in an extremity such as an arm, leg, foot, hand, toe, or finger. Exemplary ulcers have a diameter of at least about 25 mm, 50 mm, 1 cm, 2 cm, 3 cm, 4 cm, 5 cm, 6 cm, 7 cm, 8 cm, 9 cm, 10 cm, or greater.

Routes of administration of the hydrogel include injection or implantation, e.g., subcutaneously, intramuscularly, or intravenously. Alternate routes of hydrogel administration, e.g., in the case of a wound dressing, include topical application, e.g., applying the hydrogel in the form of a coating, covering, dressing, or bandage contacting a wound. Other routes of administration comprise spraying the hydrogel onto a wound, e.g., as a fluid or aerosol, followed by solidification of the hydrogel once in contact with the wound. For example, the hydrogel is applied on/in an injured tissue, e.g., on, around, or in a wound.

The hydrogels of the invention have certain advantages. For most material systems available before the invention, bulk stiffness could be controlled by increasing or decreasing the polymer concentration, but this also changes the scaffold architecture and porosity. Thus, stiffness could not be controlled independently of architecture or porosity. Other previously available material systems allowed for independent control of stiffness but lacked a naturally occurring extracellular matrix element that is required to closely mimic the biological tissue microenvironment.

In contrast, the hydrogels described herein comprise an interpenetrating network (IPN) of two polymers (e.g., collagen-I and alginate) that are not covalently bonded but fully interconnected. This physical property permits the decoupling of the effects of gel stiffness from gel architecture, porosity, and adhesion ligand density. The ability to decouple these variables in gel structure allow for ease of manufacture and customizability. The ability to tune only stiffness of a hydrogel without at the same time changing gel architecture, porosity, and/or adhesion ligand density allows for the determination of aspects of cellular behavior caused solely by changes in stiffness. Also, both polymers, collagen-I and alginate, are biocompatible, biodegradable and widely used in the tissue engineering field. Moreover, the ability for the hydrogels described herein to promote the healing of tissues without the addition of drugs, e.g., soluble factors such as anti-inflammatory agents, in or on the hydrogels, allows for the hydrogels to be used as medical devices instead of drugs. By not including drugs, e.g., soluble factors, in/on the hydrogels, the desired biological/medical effect of the hydrogel is focused on a local area, e.g., on a local population of cells, as opposed to systemic release. By localizing the effect to a target site and not causing systemic effects through the body, the hydrogels result in limited adverse side effects. For example, the changes in the mechanical properties of a given wound dressing would be localized, exclusively sensed by cells in/on or recruited to the wound site and optionally infiltrating the wound dressing, therefore having minimal adverse effects to other tissues/cells in the body. In some cases, the hydrogels can be incorporated into/onto existing wound dressings that are FDA approved or commercialized but that lack the advantageous properties that the hydrogels provide.

The hydrogels described herein can be used in concert with biomaterial-based spatiotemporal control over the presentation of bioactive molecules, growth factor or cells. However, unlike previously available systems, solely tuning the stiffness of the hydrogel, e.g., in a wound dressing material, is sufficient to significantly enhance the wound healing response.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and are not intended to be limiting.

Other features and advantages of the invention will be apparent from the following detailed description and claims.

### BRIEF DESCRIPTION OF THE DRAWINGS

FIG. 1, Panels A and B show an analysis of microarchitecture of interpenetrating networks of alginate and collagen-I reveals intercalation of the polymer networks. FIG. 1, Panel A shows a scanning electron micrograph (SEM) of a hydrogel composed of alginate only, a hydrogel composed of collagen-I only and an interpenetrating network of alginate and collagen-I at the same polymer concentrations as hydrogels containing only one of the polymers. Scale bar is 2 μm. FIG. 1, Panel B shows that, using C, O, and K as internal standards, energy dispersive spectroscopy (EDS) was used to qualitatively detect different degrees of Ca incorporation within alginate/collagen-I IPNs at three different levels of calcium crosslinking. A composite EDS spectra is included as an inset.

FIG. 2, Panels A-D show that interpenetrating networks of alginate and collagen-I demonstrate no microscale phase separation nor differences in gel porosity as calcium crosslinking is varied. FIG. 2, Panel A shows a histogram of fluorescently labeled alginate intensity per pixel taken from 2 independent images of hydrogels at two different levels of calcium crosslinking. FIG. 2, Panel B shows a histogram of fast green staining intensity per pixel taken from 4 independent images of hydrogels at two different levels of calcium crosslinking. The presence of a single peak in both histograms demonstrates that there is no micro-scale phase separation in the interpenetrating networks. FIG. 2, Panel C shows a representative micrograph of confocal immunofluorescence imaging of collagen-I antibody staining of a cross-section of alginate/collagen-I interpenetrating network. Scale bars are 100 μm. FIG. 2, Panel D shows the diffusion coefficient of fluorescently labeled 70 kDa dextran as a function of calcium crosslinking in interpenetrating networks. Differences are not statistically significant (n.s.) (One-Way Anova test, p>0.05). Data is shown as mean and standard deviation of three independent experiments.

FIG. 3, Panels A-B show the storage modulus of interpenetrating networks of alginate and collagen-I can be modulated by the extent of calcium crosslinking. FIG. 3, Panel A shows frequency dependent rheology of interpenetrating networks at the indicated concentrations of calcium crosslinker, after gelation was completed. Data is representative of at least three measurements for each condition. FIG. 3, Panel B shows storage modulus at 1 Hz as a function of extent of calcium crosslinking in interpenetrating networks. Data is shown as mean and standard deviation (n=3-5).

FIG. 4, Panels A-C show that different storage moduli lead to dramatic changes in cell morphology, without affecting cell viability or collagen-I integrin receptor expression. FIG. 4, Panel A shows representative micrographs of confocal immunofluorescence imaging of the cell cytoskeleton, as shown by fluorescent F-actin staining, in cross-sections of alginate/collagen-I interpenetrating networks with storage modulus of 50 and 1200 Pa. DAPI staining is shown in blue. Scale bar is 100 μm. FIG. 4, Panel B shows a flow cytometry analysis of viability of cells recovered from interpenetrating networks crosslinked at varying calcium concentrations (n=7-10). FIG. 4, Panel C shows a flow cytometry analysis of β1-integrin antibody staining of cells recovered from interpenetrating networks crosslinked with varying concentrations of calcium (n=3). Differences are not statistically significant (n.s.) (Student's t test, p>0.05). Data is shown as mean and standard deviation in all plots. All data was collected after cells were encapsulated for 48 hours.

FIG. 5, Panels A-C show that different storage moduli promotes different wound healing genetic programs, leading to up-regulation of inflammation mediators IL10 and COX2. FIG. 5, Panel A shows the up- or down-regulation of mRNA expression of fifteen genes involved in the wound healing response by cells encapsulated in interpenetrating networks with storage modulus of 50 or 1200 Pa. Data is shown as fold-change in stiff versus soft matrices (n=3) (Student's t test, *p<0.05). FIG. 5, Panel B shows IL10 production by cells encapsulated in interpenetrating networks with storage modulus of 50 or 1200 Pa. Data is shown as fold-change in stiff versus soft matrices (n=4-6) (Student's t test, ***p<0.01). FIG. 5, Panel C shows COX2 antibody staining of cells recovered from interpenetrating networks with storage modulus of 50 and 1200 Pa (n=3) (Student's t test, *p<0.05). Data is shown as mean and standard deviation. All data was collected after cells were encapsulated for 48 hours.

FIG. 6, Panels A-B show that no microscale phase separation was observed between both polymeric meshes within the interpenetrating networks of alginate and collagen-I. FIG. 6, Panel A shows representative micrographs of confocal fluorescence imaging of FITC-labeled alginate in interpenetrating networks crosslinked with 2.44 mM (a) and 9.76 mM (b) of calcium. FIG. 6, Panel B shows representative micrographs of confocal fluorescence imaging of fast green staining of protein content in interpenetrating networks crosslinked with 2.44 mM (a) and 9.76 mM (b) of calcium.

FIG. 7 shows the gelation time course for interpenetrating networks at the indicated concentrations of calcium crosslinker. Rheology measurements showed that gelation of the interpenetrating network was completed within 40 to 50 minutes at 37° C. Storage modulus at 1 Hz is shown.

FIG. 8, Panels A-E show that cell spreading inside interpenetrating networks is not dependent on calcium concentration or number of cell adhesion ligands. FIG. 8, Panel A shows representative micrograph of fluorescence imaging of cell viability as shown by fluorescent calcein green staining of cells encapsulated in an interpenetrating network with storage modulus of 50 Pa, after 5 days of culture. Cells are able to contract and collapse the matrix. FIG. 8, Panel B shows representative brightfield image of cells encapsulated within a hydrogel composed of collagen-I only, but with 9.76 mM of CaSO4 incorporated within the matrix. Cells fully spread demonstrating that it is not the presence of calcium that inhibits cell spreading once encapsulated within the stiffer interpenetrating networks. FIG. 8, Panel C shows number of cells recovered from interpenetrating networks crosslinked with calcium at different extents. Differences are not statistically significant (n.s.) (Student's t test, p>0.05), suggesting that cells proliferate at similar rates independent of the matrix storage modulus (n=7-10). Data is shown as mean and standard deviation. Data was collected after cells were encapsulated for 48 hours. FIG. 8, Panel D shows representative histograms of flow cytometry analysis of cells recovered from interpenetrating networks crosslinked with calcium to different extents and stained for β1-integrin. Gate shown represent <1% of positive signal for the isotype control. FIG. 8, Panel E shows representative brightfield image of cells encapsulated within an interpenetrating network with storage modulus of 1200 Pa decorated with RGD binding peptides. Cells remain spherical demonstrating that the number of adhesion sites is not a limiting factor for cells to spread once encapsulated within the stiffer interpenetrating networks. Scale bars are 100 μm.

FIG. 9, Panels A-B show that enhanced matrix stiffness promotes up-regulation of inflammation mediator COX2. FIG. 9, Panel A shows representative histograms of indirect intracellular flow cytometry analysis of cells recovered from interpenetrating networks crosslinked with calcium to different extents and stained for COX2. Gate shown represent <1% of positive signal for the unstained control. FIG. 9, Panel B shows COX2 antibody staining of cells recovered from interpenetrating networks with storage modulus of 50 and 1200 Pa. (n=3) (Student's t test, ***p<0.01). Data is shown as mean and standard deviation. All data was collected after cells were encapsulated for 48 hours.

FIG. 10 is a schematic illustrating the varying stiffnesses of substrates that lead to mesenchymal stem cell differentiation into various tissue types.

### DETAILED DESCRIPTION OF THE INVENTION

Biologically inert polymer hydrogels have been developed that are composed of alginate (Huebsch et al. Nature materials. 2010; 9:518-26), hyaluronic acid (Khetan et al. Nature materials. 2013; 12:458-65), and polyethylene glycol (Peyton et al. Biomaterials. 2006; 27:4881-93), which allow one to present adhesion ligands while independently tuning matrix stiffness. However, these systems lack a naturally occurring extracellular matrix element that may be required to closely mimic the biological tissue microenvironment. To better understand the mechanisms of cellular mechanosensing, new material systems that combine the complex physical features of natural matrices with the tunability of synthetic matrices (for independent control of mechanical and adhesive properties) have been emerging in the field (Trappmann et al. Current Opinion in Biotechnology. 2013; 24:948-53). IPNs of two different polymers where one is responsible for tuning mechanical properties, and other presents extracellular matrix signals, have been described (Park et al. Biomaterials. 2003; 24:893-900; Schmidt et al. Acta Biomaterialia. 2009; 5:2385-97; Akpalo et al. Acta Biomaterialia. 2011; 7:2418-27; Sun et al. Soft matter. 2012; 8:2398-404; Tong et al. Biomaterials. 2014; 35:1807-15).

In these material systems, increasing or decreasing the polymer concentration tunes the bulk stiffness, but also changes the scaffold architecture and porosity. For example, the mechanical properties of collagen-I containing IPNs have been tuned by adding various quantities of agarose (Ulrich et al. Biomaterials. 2010; 31:1875-84). Thus, in these previously described systems, stiffness cannot be tuned independently of scaffold architecture and porosity.

In another approach, a gelatin network was crosslinked by transglutaminase and an intercalated alginate network crosslinked by calcium ions (Wen et al. Macromolecular Materials and Engineering. 2013). However, the impact of solely changing the extent of calcium crosslinking in that system was not investigated.

The invention features a biomaterial system, e.g., hydrogel, made up of interpenetrating networks (IPNs) of alginate and collagen (e.g., collagen-I) that decouple the effects of gel stiffness from gel architecture, porosity and adhesion ligand density. As described in detail in the Examples, characterization of the microarchitecture of the alginate/collagen IPNs revealed that the degree of Ca⁺² crosslinking did not change gel porosity or architecture, when the polymer concentration in the system remained constant. The alginate/collagen IPNs had viscoelastic behavior similar to skin, which adapts its internal collagen meshwork structure when stretched in order to minimize strain (Edwards et al. Clinics in Dermatology. 1995; 13:375-80). The storage modulus of the IPNs was tuned from 50 to 1200 Pascal (Pa) by controlling the extent of crosslinking with calcium divalent cations (Ca⁺²), within ranges that are compatible with cell viability. Macromolecular transport studies demonstrated that diffusion of small metabolites was not affected by the extent of crosslinking of the alginate component, consistent with previous studies on alginate gels (Huebsch et al. Nature Materials. 2010; 9:518-26).

Thus, included in the invention is a 3-dimensional hydrogel comprising an interpenetrating network of alginate and collagen, wherein the hydrogel comprises a storage modulus of 20 Pa or greater, e.g., 20, 30, 40, 50, 60, 70, 80, 90, 100, 150, 200, 250, 300, 400, 500, 600, or 800 Pa, 1, 2, 3, 4, 5, 10, 50, 100, 500 kPa, 1, 2, 3, 4, 5, 10, 50, 100, or 500 MPa, or greater. In some cases, the storage modulus is between 50 kPa and 50 MPa. In some examples, the storage modulus is between 30 Pa and 1200 Pa For example, the storage modulus is between 30 Pa and 400 Pa, (e.g., 400, 300, 250, 200, 150, 100, 75, 60, 55, 50, 45, 40, 35, or 30 Pa) or between 30 Pa and 300 Pa.

Also included in the invention is a 3-dimensional hydrogel comprising an interpenetrating network of alginate and MATRIGEL™, wherein the hydrogel comprises a storage modulus of 20 Pa or greater, e.g., 20, 30, 40, 50, 60, 70, 80, 90, 100, 150, 200, 250, 300, 400, 500, 600, or 800 Pa, 1, 2, 3, 4, 5, 10, 50, 100, 500 kPa, 1, 2, 3, 4, 5, 10, 50, 100, or 500 MPa, or greater. In some cases, the storage modulus is between 50 kPa and 50 MPa. In some examples, the storage modulus is between 30 Pa and 1200 Pa For example, the storage modulus is between 30 Pa and 400 Pa, (e.g., 400, 300, 250, 200, 150, 100, 75, 60, 55, 50, 45, 40, 35, or 30 Pa) or between 30 Pa and 300 Pa.

For example, MATRIGEL™ comprises a mixture of extracellular matrix proteins, e.g., laminin 111 and collagen IV. Laminin 111 binds to α6β4 integrin. See, e.g., Niessen et al. Exp. Cell Res. 211(1994):360-367. For example, the IPNs are made of a concentration of about 3-6 mg/mL (e.g., about 4, or about 4.4 mg/mL) MATRIGEL™ (available from BD Biosciences) and about 3-7 mg/mL (e.g., about 5 mg/mL) alginate.

In some cases, the IPNs described herein present a constant number of adhesion sites, since the alginate backbone presents no binding motifs to which cells can adhere and the concentration of collagen (e.g., collagen-I) remains constant. In some examples, these IPNs are prone to cellular-mediated matrix cleavage and remodel across time. The data presented herein described the first 48 hours of cell culture.

The hydrogels of the invention have certain effects on the biology and behavior of cells. For example, adult dermal fibroblasts showed dramatic differences in cell morphology once encapsulated in alginate/collagen IPNs of various moduli. The cells spread extensively in soft substrates, but remained round in IPNs of higher stiffness. Cells probe mechanical properties as they adhere and pull on their surroundings, but also dynamically reorganize their cytoskeleton in response to the resistance that they feel (Discher et al. Science 2005; 310:1139-43). Fibroblasts sense and respond to the compliance of their substrate (Jerome et al. Biophysical Journal. 2007; 93:4453-61). Most studies, however, have been performed in two-dimensional substrates, and there is increasing evidence that adhesions between fibroblasts and extracellular matrix are considerably different in three-dimensional cultures (Cukierman et al. Science 2001; 294:1708-12). In the three-dimensional alginate/collagen IPN, fibroblasts failed to form stress fibers on stiffer matrices, likely because the resistance to deformation was higher than cellular traction forces. The failure of the cells to spread even as the alginate polymeric backbone was further decorated with RGD binding sites in stiffer matrices shows that, in some cases, the ability of fibroblasts to elongate and deform the surrounding matrix is controlled by their cell traction forces and not by cell binding site density. The results presented herein show that the morphology and contractility of fibroblasts infiltrating a wound dressing can be modulated simply by controlling the storage modulus of the biomaterial itself.

Tuning the storage modulus of the alginate/collagen interpenetrating network also induced different wound healing-related genetic profiles in dermal fibroblasts, with differential expression of genes related to inflammatory cascades, collagen synthesis, surface adhesion receptors and extracellular matrix molecules. For example, CCL2 is downregulated in fibroblasts encapsulated in stiffer matrices. Fibroblasts activate intracellular focal adhesion kinases (FAK) following cutaneous injury, and FAK acts through extracellular-related kinase (ERK) to trigger the secretion of CCL2 (Victor et al. Nature Medicine. 2011; 18:148-52). The failure of fibroblasts to spread in stiffer alginate/collagen IPNs is consistent with the down-regulated expression of CCL2. Also, COX2 and IL10 are up-regulated in fibroblasts on stiffer matrices. COX2 is responsible for the elevated production of prostanoids in sites of disease and inflammation (Warner et al. FASEB Journal. 2004; 18:790-804). IL10 has a central role in regulating the cytokine network behind inflammation, and also regulates COX2 during acute inflammatory responses (Berg et al. Journal of Immunology. 2001; 166:2674-80). As inflammation is a key aspect of wound healing (Eming et al. J Invest Dermatol. 2007; 127:514-25), the ability of a wound dressing material to induce or suppress the expression of key orchestrators of inflammation such as IL10 and COX2 is useful to guide the outcome of the healing cascade.

GenBank Accession Nos. of proteins and nucleic acid molecules described herein are presented below.

The mRNA sequence of human interleukin 10 (IL10) is provided by GenBank Accession No. NM_000572.2, incorporated herein by reference, which is shown below (SEQ ID NO: 1). The start and stop codons are shown in bold and underlined font.

| (SEQ ID NO: 1) | |
|---|---|
| 1 | acacatcagg ggcttgctct tgcaaaacca aaccacaaga cagacttgca aaagaaggca |
| 61 | tgcacaqctc agcactgctc tgttgcctgg tcctcctgac tggggtgagg gccagcccag |
| 121 | gccagggcac ccagtctgag aacagctgca cccacttccc aggcaacctg cctaacatgc |
| 181 | ttcgagatct ccgagatgcc ttcagcagag tgaagacttt ctttcaaatg aaggatcagc |
| 241 | tggacaactt gttgttaaag gagtccttgc tggaggactt taagggttac ctgggttgcc |
| 301 | aagccttgtc tgagatgatc cagttttacc tggaggaggt gatgccccaa gctgagaacc |
| 361 | aagacccaga catcaaggcg catgtgaact ccctggggga gaacctgaag accctcaggc |
| 421 | tgaggctacg gcgctgtcat cgatttcttc cctgtgaaaa caagagcaag gccgtggagc |
| 481 | aggtgaagaa tgcctttaat aagctccaag agaaaggcat ctacaaagcc atgagtgagt |
| 541 | ttgacatctt catcaactac atagaagcct acatgacaat gaagatacga aactgagaca |
| 601 | tcagggtggc gactctatag actctaggac ataaattaga ggtctccaaa atcggatctg |
| 661 | gggctctggg atagctgacc cagccccttg agaaacctta ttgtacctct cttatagaat |
| 721 | atttattacc tctgatacct caacccccat ttctatttat ttactgagct tctctgtgaa |
| 781 | cgatttagaa agaagcccaa tattataatt tttttcaata tttattattt tcacctgttt |
| 841 | ttaagctgtt tccatagggt gacacactat ggtatttgag tgttttaaga taaattataa |
| 901 | gttacataag ggaggaaaaa aaatgttctt tggggagcca acagaagctt ccattccaag |
| 961 | cctgaccacg ctttctagct gttgagctgt tttccctgac ctccctctaa tttatcttgt |
| 1021 | ctctgggctt ggggcttcct aactgctaca aatactctta ggaagagaaa ccagggagcc |
| 1081 | cctttgatga ttaattcacc ttccagtgtc tcggagggat tcccctaacc tcattcccca |
| 1141 | accacttcat tcttgaaagc tgtggccagc ttgttattta taacaaccta aatttggttc |
| 1201 | taggccgggc gcggtggctc acgcctgtaa tcccagcact ttgggaggct gaggcgggtg |
| 1261 | gatcacttga ggtcaggagt tcctaaccag cctggtcaac atggtgaaac cccgtctcta |
| 1321 | ctaaaaatac aaaaattagc cgggcatggt ggcgcgcacc tgtaatccca gctacttggg |
| 1381 | aggctgaggc aagagaattg cttgaaccca ggagatggaa gttgcagtga gctgatatca |
| 1441 | tgcccctgta ctccagcctg ggtgacagag caagactctg tctcaaaaaa taaaaataaa |
| 1501 | aataaatttg gttctaatag aactcagttt taactagaat ttattcaatt cctctgggaa |
| 1561 | tgttacattg tttgtctgtc ttcatagcag attttaattt tgaataaata aatgtatctt |
| 1621 | attcacatc |

The amino acid sequence of human IL-10 is provided by GenBank Accession No. NP_000563.1, incorporated herein by reference, which is shown below (SEQ ID NO: 2). The signal peptide is shown in underlined font, and the mature peptide is shown in italicized font.

| (SEQ ID NO: 2) | |
|---|---|
| 1 | mhssallccl vlltgvrasp gqgtqsensc thfpgnlpnm lrdlrdafsr vktffqmkdq |
| 61 | ldnlllkesl ledfkgylgc qalsemiqfy leevmpqaen qdpdikahvn slgenlktlr |
| 121 | lrlrrchrfl pcenkskave qvknafnklq ekgiykamse fdifinyiea ymtmkirn |

The mRNA sequence of human prostaglandin-endoperoxide synthase 2 (PTGS2) (also known as COX2) is provided by GenBank Accession No. NM_000963.3, incorporated herein by reference, which is shown below (SEQ ID NO: 3). The start and stop codons are shown in bold and underlined font.

| (SEQ ID NO: 3) | |
|---|---|
| 1 | gaccaattgt catacgactt gcagtgagcg tcaggagcac gtccaggaac tcctcagcag |
| 61 | cgcctccttc agctccacag ccagacgccc tcagacagca aagcctaccc ccgcgccgcg |
| 121 | ccctgcccgc cgctgcgatg ctcgcccgcg ccctgctgct gtgcgcggtc ctggcgctca |
| 181 | gccatacagc aaatccttgc tgttcccacc catgtcaaaa ccgaggtgta tgtatgagtg |
| 241 | tgggatttga ccagtataag tgcgattgta cccggacagg attctatgga gaaaactgct |
| 301 | caacaccgga atttttgaca agaataaaat tatttctgaa acccactcca aacacagtgc |
| 361 | actacatact tacccacttc aagggatttt ggaacgttgt gaataacatt cccttccttc |
| 421 | gaaatgcaat tatgagttat gtgttgacat ccagatcaca tttgattgac agtccaccaa |
| 481 | cttacaatgc tgactatggc tacaaaagct gggaagcctt ctctaacctc tcctattata |
| 541 | ctagagccct tcctcctgtg cctgatgatt gcccgactcc cttgggtgtc aaaggtaaaa |
| 601 | agcagcttcc tgattcaaat gagattgtgg aaaaattgct tctaagaaga aagttcatcc |
| 661 | ctgatcccca gggctcaaac atgatgtttg cattctttgc ccagcacttc acgcatcagt |
| 721 | ttttcaagac agatcataag cgagggccag ctttcaccaa cgggctgggc catggggtgg |
| 781 | acttaaatca tatttacggt gaaactctgg ctagacagcg taaactgcgc cttttcaagg |
| 841 | atggaaaaat gaaatatcag ataattgatg gagagatgta tcctcccaca gtcaaagata |
| 901 | ctcaggcaga gatgatctac cctcctcaag tccctgagca tctacggttt gctgtggggc |
| 961 | aggaggtctt tggtctggtg cctggtctga tgatgtatgc cacaatctgg ctgcgggaac |
| 1021 | acaacagagt atgcgatgtg cttaaacagg agcatcctga atggggtgat gagcagttgt |
| 1081 | tccagacaag caggctaata etgataggag agactattaa gattgtgatt gaagattatg |
| 1141 | tgcaacactt gagtggctat cacttcaaac tgaaatttga cccagaacta cttttcaaca |
| 1201 | aacaattcca gtaccaaaat cgtattgctg ctgaatttaa caccctctat cactggcatc |
| 1261 | cccttctgcc tgacaccttt caaattcatg accagaaata caactatcaa cagtttatct |
| 1321 | acaacaactc tatattgctg gaacatggaa ttacccagtt tgttgaatca ttcaccaggc |
| 1381 | aaattgctgg cagggttgct ggtggtagga atgttccacc cgcagtacag aaagtatcac |
| 1441 | aggcttccat tgaccagagc aggcagatga aataccagtc ttttaatgag taccgcaaac |
| 1501 | gctttatgct gaagccctat gaatcatttg aagaacttac aggagaaaag gaaatgtctg |
| 1561 | cagagttgga agcactctat ggtgacatcg atgctgtgga gctgtatcct gcccttctgg |
| 1621 | tagaaaagcc tcggccagat gccatctttg gtgaaaccat ggtagaagtt ggagcaccat |
| 1681 | tctccttgaa aggacttatg ggtaatgtta tatgttctcc tgcctactgg aagccaagca |
| 1741 | cttttggtgg agaagtgggt tttcaaatca tcaacactgc ctcaattcag tctctcatct |
| 1801 | gcaataacgt gaagggctgt ccctttactt cattcagtgt tccagatcca gagctcatta |
| 1861 | aaacagtcac catcaatgca agttcttccc gctccggact agatgatatc aatcccacag |
| 1921 | tactactaaa agaacgttcg actgaactgt agaagtctaa tgatcatatt tatttattta |
| 1981 | tatgaaccat gtctattaat ttaattattt aataatattt atattaaact ccttatgtta |
| 2041 | cttaacatct tctgtaacag aagtcagtac tcctgttgcg gagaaaggag tcatacttgt |
| 2101 | gaagactttt atgtcactac tctaaagatt ttgctgttgc tgttaagttt ggaaaacagt |
| 2161 | ttttattctg ttttataaac cagagagaaa tgagttttga cgtcttttta cttgaatttc |
| 2221 | aacttatatt ataagaacga aagtaaagat gtttgaatac ttaaacactg tcacaagatg |
| 2281 | gcaaaatgct gaaagttttt acactgtcga tgtttccaat gcatcttcca tgatgcatta |
| 2341 | gaagtaacta atgtttgaaa ttttaaagta cttttggtta tttttctgtc atcaaacaaa |
| 2401 | aacaggtatc agtgcattat taaatgaata tttaaattag acattaccag taatttcatg |
| 2461 | tctacttttt aaaatcagca atgaaacaat aatttgaaat ttctaaattc atagggtaga |
| 2521 | atcacctgta aaagcttgtt tgatttctta aagttattaa acttgtacat ataccaaaaa |
| 2581 | gaagctgtct tggatttaaa tctgtaaaat cagtagaaat tttactacaa ttgcttgtta |
| 2641 | aaatatttta taagtgatgt tcctttttca ccaagagtat aaaccttttt agtgtgactg |
| 2701 | ttaaaacttc cttttaaatc aaaatgccaa atttattaag gtggtggagc cactgcagtg |
| 2761 | ttatcttaaa ataagaatat tttgttgaga tattccagaa tttgtttata tggctggtaa |
| 2821 | catgtaaaat ctatatcagc aaaagggtct acctttaaaa taagcaataa caaagaagaa |
| 2881 | aaccaaatta ttgttcaaat ttaggtttaa acttttgaag caaacttttt tttatccttg |
| 2941 | tgcactgcag gcctggtact cagattttgc tatgaggtta atgaagtacc aagctgtgct |
| 3001 | tgaataatga tatgttttct cagattttct gttgtacagt ttaatttagc agtccatatc |
| 3061 | acattgcaaa agtagcaatg acctcataaa atacctcttc aaaatgctta aattcatttc |
| 3121 | acacattaat tttatctcag tcttgaagcc aattcagtag gtgcattgga atcaagcctg |
| 3181 | gctacctgca tgctgttcct tttcttttct tcttttagcc attttgctaa gagacacagt |
| 3241 | cttctcatca cttcgtttct cctattttgt tttactagtt ttaagatcag agttcacttt |
| 3301 | ctttggactc tgcctatatt ttcttacctg aacttttgca agttttcagg taaacctcag |
| 3361 | ctcaggactg ctatttagct cctcttaaga agattaaaag agaaaaaaaa aggccctttt |
| 3421 | aaaaatagta tacacttatt ttaagtgaaa agcagagaat tttatttata gctaatttta |
| 3481 | gctatctgta accaagatgg atgcaaagag gctagtgcct cagagagaac tgtacggggt |
| 3541 | ttgtgactgg aaaaagttac gttcccattc taattaatgc cctttcttat ttaaaaacaa |
| 3601 | aaccaaatga tatctaagta gttctcagca ataataataa tgacgataat acttcttttc |
| 3661 | cacatctcat tgtcactgac atttaatggt actgtatatt acttaattta ttgaagatta |
| 3721 | ttatttatgt ettattagga cactatggtt ataaactgtg tttaagccta caatcattga |
| 3781 | tttttttttg ttatgtcaca atcagtatat tttctttggg gttacctctc tgaatattat |
| 3841 | gtaaacaatc caaagaaatg attgtattaa gatttgtgaa taaattttta gaaatctgat |
| 3901 | tggcatattg agatatttaa ggttgaatgt ttgtccttag gataggccta tgtgctagcc |
| 3961 | cacaaagaat attgtctcat tagcctgaat gtgccataag actgaccttt taaaatgttt |
| 4021 | tgagggatct gtggatgctt cgttaatttg ttcagccaca atttattgag aaaatattct |
| 4081 | gtgtcaagca ctgtgggttt taatattttt aaatcaaacg ctgattacag ataatagtat |
| 4141 | ttatataaat aattgaaaaa aattttcttt tgggaagagg gagaaaatga aataaatatc |
| 4201 | attaaagata actcaggaga atcttcttta caattttacg tttagaatgt ttaaggttaa |
| 4261 | gaaagaaata gtcaatatgc ttgtataaaa cactgttcac tgtttttttt aaaaaaaaaa |
| 4321 | cttgatttgt tattaacatt gatctgctga caaaacctgg gaatttgggt tgtgtatgcg |
| 4381 | aatgtttcag tgcctcagac aaatgtgtat ttaacttatg taaaagataa gtctggaaat |
| 4441 | aaatgtctgt ttatttttgt actatttaaa aattgacaga tcttttctga agaaaaaaaa |
| 4501 | aaaaaaa |

The amino acid sequence of human prostaglandin-endoperoxide synthase 2 (PTGS2) (also known as COX2) is provided by GenBank Accession No. NP_000954.1, incorporated herein by reference, which is shown below (SEQ ID NO: 4). The predicted signal peptide is shown in underlined font.

| (SEO ID NO: 4) | |
|---|---|
| 1 | mlaralllca vlalshtanp ccshpcqnrg vemsvgfdqy kcdctrtgfy gencstpefl |
| 61 | triklflkpt pntvhyilth fkgfwnvvnn ipflrnaims yvltsrshli dspptynady |
| 121 | gyksweafsn lsyytralpp vpddcptplg vkgkkqlpds neiveklllr rkfipdpqgs |
| 181 | nmmfaffaqh fthqffktdh krgpaftngl ghgvdlnhiy getlarqrkl rlfkdgkmky |
| 241 | qiidgemypp tvkdtqaemi yppqvpehlr favgqevfgl vpglmmyati wlrehnrvcd |
| 301 | vlkqehpewg deqlfqtsrl iligetikiv iedyvqhlsg yhfklkfdpe llfnkqfqyq |
| 361 | nriaaefntl yhwhpllpdt fqihdqkyny qqfiynnsil lehgitqfve sftrqiagrv |
| 421 | aggrnvppav qkvsqasidq srqmkyqsfn eyrkrfmlkp yesfeeltge kemsaeleal |
| 481 | ygdidavely pallvekprp daifgetmve vgapfslkgl mgnvicspay wkpstfggev |
| 541 | gfqiintasi qslicnnvkg cpftsfsvpd peliktvtin asssrsgldd inptvllker |
| 601 | stel |

The mRNA sequence of human integrin a4 (ITGA4) is provided by GenBank Accession No. NM_000885.4 and is shown below (SEQ ID NO: 5). The start and stop codons are bolded and underlined.

| (SEQ ID NO: 5) | |
|---|---|
| 1 | ataacgtctt tgtcactaaa atgttcccca ggggccttcg gcgagtcttt ttgtttggtt |
| 61 | ttttgttttt aatctgtggc tcttgataat ttatctagtg gttgcctaca cctgaaaaac |
| 121 | aagacacagt gtttaactat caacgaaaga actggacggc tccccgccgc agtcccactc |
| 181 | cccgagtttg tggctggcat ttgggccacg ccgggctggg cggtcacagc gaggggcgcg |
| 241 | cagtttgggg tcacacagct ccgcttctag gccccaacca ccgttaaaag gggaagcccg |
| 301 | tgccccatca ggtccgctct tgctgagccc agagccatcc cgcgctctgc gggctgggag |
| 361 | gcccgggcca ggacgcgagt cctgcgcagc cgaggttccc cagcgccccc tgcagccgcg |
| 421 | cgtaggcaga gacggagccc ggccctgcgc ctccgcacca cgcccgggac cccacccagc |
| 481 | ggcccgtacc cggagaagca gcgcgagcac ccgaagctcc cggctggcgg cagaaaccgg |
| 541 | gagtggggcc gggcgagtgc gcggcatccc aggccggccc gaacgctccg cccgcggtgg |
| 601 | gccgacttcc cctcctcttc cctctctcct tcctttagcc cgctggcgcc ggacacgctg |
| 661 | cgcctcatct cttggggcgt tcttccccgt tggccaaccg tcgcatcccg tgcaactttg |
| 721 | gggtagtggc cgtttagtgt tgaatgttcc ccaccgagag cgcatggctt gggaagcgag |
| 781 | gcgcgaaccc ggcccccgaa gggccgccgt ccgggagacg gtgatgctgt tgctgtgcct |
| 841 | gggggtcccg accggccgcc cctacaacgt ggacactgag agcgcgctgc tttaccaggg |
| 901 | cccccacaac acgctgttcg gctactcggt cgtgctgcac agccacgggg cgaaccgatg |
| 961 | gctcctagtg ggtgcgccca ctgccaactg gctcgccaac gcttcagtga tcaatcccgg |
| 1021 | ggcgatttac agatgcagga tcggaaagaa tcccggccag acgtgcgaac agctccagct |
| 1081 | gggtagccct aatggagaac cttgtggaaa gacttgtttg gaagagagag acaatcagtg |
| 1141 | gttgggggtc acactttcca gacagccagg agaaaatgga tccatcgtga cttgtgggca |
| 1201 | tagatggaaa aatatatttt acataaagaa tgaaaataag ctccccactg gtggttgcta |
| 1261 | tggagtgccc cctgatttac gaacagaact gagtaaaaga atagctccgt gttatcaaga |
| 1321 | ttatgtgaaa aaatttggag aaaattttgc atcatgtcaa gctggaatat ccagttttta |
| 1381 | cacaaaggat ttaattgtga tgggggcccc aggatcatct tactggactg gctctctttt |
| 1441 | tgtctacaat ataactacaa ataaatacaa ggctttttta gacaaacaaa atcaagtaaa |
| 1501 | atttggaagt tatttaggat attcagtcgg agctggtcat tttcggagcc agcatactac |
| 1561 | cgaagtagtc ggaggagctc ctcaacatga gcagattggt aaggcatata tattcagcat |
| 1621 | tgatgaaaaa gaactaaata tcttacatga aatgaaaggt aaaaagcttg gatcgtactt |
| 1681 | tggagcttct gtctgtgctg tggacctcaa tgcagatggc ttctcagatc tgctcgtggg |
| 1741 | agcacccatg cagagcacca tcagagagga aggaagagtg tttgtgtaca tcaactctgg |
| 1801 | ctcgggagca gtaatgaatg caatggaaac aaacctcgtt ggaagtgaca aatatgctgc |
| 1861 | aagatttggg gaatctatag ttaatcttgg cgacattgac aatgatggct ttgaagatgt |
| 1921 | tgctatcgga gctccacaag aagatgactt gcaaggtgct atttatattt acaatggccg |
| 1981 | tgcagatggg atctcgtcaa ccttctcaca gagaattgaa ggacttcaga tcagcaaatc |
| 2041 | gttaagtatg tttggacagt ctatatcagg acaaattgat gcagataata atggctatgt |
| 2101 | agatgtagca gttggtgctt ttcggtctga ttctgctgtc ttgctaagga caagacctgt |
| 2161 | agtaattgtt gacgcttctt taagccaccc tgagtcagta aatagaacga aatttgactg |
| 2221 | tgttgaaaat ggatggcctt ctgtgtgcat agatctaaca ctttgtttct catataaggg |
| 2281 | caaggaagtt ccaggttaca ttgttttgtt ttataacatg agtttggatg tgaacagaaa |
| 2341 | ggcagagtct ccaccaagat tctatttctc ttctaatgga acttctgacg tgattacagg |
| 2401 | aagcatacag gtgtccagca gagaagctaa ctgtagaaca catcaagcat ttatgcggaa |
| 2461 | agatgtgcgg gacatcctca ccccaattca gattgaagct gcttaccacc ttggtcctca |
| 2521 | tgtcatcagt aaacgaagta cagaggaatt cccaccactt cagccaattc ttcagcagaa |
| 2581 | gaaagaaaaa gacataatga aaaaaacaat aaactttgca aggttttgtg cccatgaaaa |
| 2641 | ttgttctgct gatttacagg tttctgcaaa gattgggttt ttgaagcccc atgaaaataa |
| 2701 | aacatatctt gctgttggga gtatgaagac attgatgttg aatgtgtcct tgtttaatgc |
| 2761 | tggagatgat gcatatgaaa cgactctaca tgtcaaacta cccgtgggtc tttatttcat |
| 2821 | taagatttta gagctggaag agaagcaaat aaactgtgaa gtcacagata actctggcgt |
| 2881 | ggtacaactt gactgcagta ttggctatat atatgtagat catctctcaa ggatagatat |
| 2941 | tagctttctc ctggatgtga gctcactcag cagagcggaa gaggacctca gtatcacagt |
| 3001 | gcatgctacc tgtgaaaatg aagaggaaat ggacaatcta aagcacagca gagtgactgt |
| 3061 | agcaatacct ttaaaatatg aggttaagct gactgttcat gggtttgtaa acccaacttc |
| 3121 | atttgtgtat ggatcaaatg atgaaaatga gcctgaaacg tgcatggtgg agaaaatgaa |
| 3181 | cttaactttc catgttatca acactggcaa tagtatggct cccaatgtta gtgtggaaat |
| 3241 | aatggtacca aattctttta gcccccaaac tgataagctg ttcaacattt tggatgtcca |
| 3301 | gactactact ggagaatgcc actttgaaaa ttatcaaaga gtgtgtgcat tagagcagca |
| 3361 | aaagagtgca atgcagacct tgaaaggcat agtccggttc ttgtccaaga ctgataagag |
| 3421 | gctattgtac tgcataaaag ctgatccaca ttgtttaaat ttcttgtgta attttgggaa |
| 3481 | aatggaaagt ggaaaagaag ccagtgttca tatccaactg gaaggccggc catccatttt |
| 3541 | agaaatggat gagacttcag cactcaagtt tgaaataaga gcaacaggtt ttccagagcc |
| 3601 | aaatccaaga gtaattgaac taaacaagga tgagaatgtt gcgcatgttc tactggaagg |
| 3661 | actacatcat caaagaccca aacgttattt caccatagtg attatttcaa gtagcttgct |
| 3721 | acttggactt attgtacttc tgttgatctc atatgttatg tggaaggctg gcttctttaa |
| 3781 | aagacaatac aaatctatcc tacaagaaga aaacagaaga gacagttgga gttatatcaa |
| 3841 | cagtaaaagc aatgatgatt aaggacttct ttcaaattga gagaatggaa aacagactca |
| 3901 | ggttgtagta aagaaattta aaagacactg tttacaagaa aaaatgaatt ttgtttggac |
| 3961 | ttcttttact catgatcttg tgacatatta tgtcttcatg caaggggaaa atctcagcaa |
| 4021 | tgattactct ttgagataga agaactgcaa aggtaataat acagccaaag ataatctctc |
| 4081 | agcttttaaa tgggtagaga aacactaaag cattcaattt attcaagaaa agtaagccct |
| 4141 | tgaagatatc ttgaaatgaa agtataactg agttaaatta tactggagaa gtcttagact |
| 4201 | tgaaatacta cttaccatat gtgcttgcct cagtaaaatg aaccccactg ggtgggcaga |
| 4261 | ggttcatttc aaatacatct ttgatacttg ttcaaaatat gttctttaaa aatataattt |
| 4321 | tttagagagc tgttcccaaa ttttctaacg agtggaccat tatcacttta aagcccttta |
| 4381 | tttataatac atttcctacg ggctgtgttc caacaaccat tttttttcag cagactatga |
| 4441 | atattatagt attataggcc aaactggcaa acttcagact gaacatgtac actggtttga |
| 4501 | gcttagtgaa attacttctg gataattatt tttttataat tatggatttc accatctttc |
| 4561 | tttctgtata tatacatgtg tttttatgta ggtatatatt taccattctt cctatctatt |
| 4621 | cttcctataa cacaccttta tcaagcatac ccaggagtaa tcttcaaatc ttttgttata |
| 4681 | ttctgaaaca aaagattgtg agtgttgcac tttacctgat acacgctgat ttagaaaata |
| 4741 | cagaaaccat acctcactaa taactttaaa atcaaagctg tgcaaagact agggggccta |
| 4801 | tacttcatat gtattatgta ctatgtaaaa tattgactat cacacaacta tttccttgga |
| 4861 | tgtaattctt tgttaccctt tacaagtata agtgttacct tacatggaaa cgaagaaaca |
| 4921 | aaattcataa atttaaattc ataaatttag ctgaaagata ctgattcaat ttgtatacag |
| 4981 | tgaatataaa tgagacgaca gcaaaatttt catgaaatgt aaaatatttt tatagtttgt |
| 5041 | tcatactata tgaggttcta ttttaaatga ctttctggat tttaaaaaat ttctttaaat |
| 5101 | acaatcattt ttgtaatatt tattttatgc ttatgatcta gataattgca gaatatcatt |
| 5161 | ttatctgact ctgccttcat aagagagctg tggccgaatt ttgaacatct gttataggga |
| 5221 | gtgatcaaat tagaaggcaa tgtggaaaaa caattctggg aaagatttct ttatatgaag |
| 5281 | tccctgccac tagccagcca tcctaattga tgaaagttat ctgttcacag gcctgcagtg |
| 5341 | atggtgagga atgttctgag atttgcgaag gcatttgagt agtgaaatgt aagcacaaaa |
| 5401 | cctcctgaac ccagagtgtg tatacacagg aataaacttt atgacattta tgtattttta |
| 5461 | aaaaactttg tatcgttata aaaaggctag tcattctttc aggagaacat ctaggatcat |
| 5521 | agatgaaaaa tcaagccccg atttagaact gtcttctcca ggatggtctc taaggaaatt |
| 5581 | tacatttggt tctttcctac tcagaactac tcagaaacaa ctatatattt caggttatct |
| 5641 | gagcacagtg aaagcagagt actatggttg tccaacacag gcctctcaga tacaagggga |
| 5701 | acacaattac atattgggct agattttgcc cagttcaaaa tagtatttgt tatcaactta |
| 5761 | ctttgttact tgtatcatga attttaaaac cctaccactt taagaagaca gggatgggtt |
| 5821 | attctttttt ggcaggtagg ctatataact atgtgatttt gaaatttaac tgctctggat |
| 5881 | tagggagcag tgaatcaagg cagacttatg aaatctgtat tatatttgta acagaatata |
| 5941 | ggaaatttaa cataattgat gagctcaaat cctgaaaaat gaaagaatcc aaattatttc |
| 6001 | agaattatct aggttaaata ttgatgtatt atgatggttg caaagttttt ttgtgtgtcc |
| 6061 | aataaacaca ttgtaaaaaa aa |

The amino acid sequence of human ITGA4 is provided by GenBank Accession No. NP_000876.3 and is shown below (SEQ ID NO: 6). The predicted signal peptide is underlined.

| (SEQ ID NO: 6) | |
|---|---|
| 1 | mawearrepg prraavretv mlllclgvpt grpynvdtes allyqgphnt lfgysvvlhs |
| 61 | hganrwllvg aptanwlana svinpgaiyr crigknpgqt ceqlqlgspn gepcgktcle |
| 121 | erdnqwlgvt lsrqpgengs ivtcghrwkn ifyiknenkl ptggcygvpp dlrtelskri |
| 181 | apcyqdyvkk fgenfascqa gissfytkdl ivmgapgssy wtgslfvyni ttnkykafld |
| 241 | kqnqvkfgsy lgysvgaghf rsqhttevvg gapqheqigk ayifsideke lnilhemkgk |
| 301 | klgsyfgasv cavdlnadgf sdllvgapmq stireegrvf vyinsqsqav mnametnlvg |
| 361 | sdkyaarfge sivnlgdidn dgfedvaiga pqeddlqqai yiyngradgi sstfsqrieg |
| 421 | lqiskslsmf gqsisgqida dnngyvdvav gafrsdsavl lrtrpvvivd aslshpesvn |
| 481 | rtkfdcveng wpsvcidltl cfsykgkevp gyivlfynms ldvnrkaesp prfyfssngt |
| 541 | sdvitgsiqv ssreancrth qafmrkdvrd iltpiqieaa yhlgphvisk rsteefpplq |
| 601 | pilqqkkekd imkktinfar fcahencsad lqvsakigfl kphenktyla vgsmktlmln |
| 661 | vslfnagdda yettlhvklp vglyfikile leekqincev tdnsgvvqld csigyiyvdh |
| 721 | lsridisfll dvsslsraee dlsitvhatc eneeemdnlk hsrvtvaipl kyevkltvhg |
| 781 | fvnptsfvyg sndenepetc mvekmnltfh vintgnsmap nvsveimvpn sfspqtdklf |
| 841 | nildvqtttg echfenyqrv caleqqksam qtlkgivrfl sktdkrllyc ikadphclnf |
| 901 | lcnfgkmesg keasvhiqle grpsilemde tsalkfeira tgfpepnprv ielnkdenva |
| 961 | hvlleglhhq rpkryftivi issslllgli vlllisyvmw kagffkrqyk silqeenrrd |
| 1021 | swsyinsksn dd |

The mRNA sequence of human metallopeptidase 1 (MMP1) is provided by GenBank Accession No. NM_002421.3 and is shown below (SEQ ID NO: 7). The start and stop codons are underlined and bolded.

| (SEQ ID NO: 7) | |
|---|---|
| 1 | agcatgagtc agacagcctc tggctttctg gaagggcaag gactctatat atacagaggg |
| 61 | agcttcctag ctgggatatt ggagcagcaa gaggctggga agccatcact taccttgcac |
| 121 | tgagaaagaa gacaaaggcc agtatgcaca gctttcctcc actgctgctg ctgctgttct |
| 181 | ggggtgtggt gtctcacagc ttcccagcga ctctagaaac acaagagcaa gatgtggact |
| 241 | tagtccagaa atacctggaa aaatactaca acctgaagaa tgatgggagg caagttgaaa |
| 301 | agcggagaaa tagtggccca gtggttgaaa aattgaagca aatgcaggaa ttctttgggc |
| 361 | tgaaagtgac tgggaaacca gatgctgaaa ccctgaaggt gatgaagcag cccagatgtg |
| 421 | gagtgcctga tgtggctcag tttgtcctca ctgaggggaa ccctcgctgg gagcaaacac |
| 481 | atctgaccta caggattgaa aattacacgc cagatttgcc aagagcagat gtggaccatg |
| 541 | ccattgagaa agccttccaa ctctggagta atgtcacacc tctgacattc accaaggtct |
| 601 | ctgagggtca agcagacatc atgatatctt ttgtcagggg agatcatcgg gacaactctc |
| 661 | cttttgatgg acctggagga aatcttgctc atgcttttca accaggccca ggtattggag |
| 721 | gggatgctca ttttgatgaa gatgaaaggt ggaccaacaa tttcagagag tacaacttac |
| 781 | atcgtgttgc agctcatgaa ctcggccatt ctcttggact ctcccattct actgatatcg |
| 841 | gggctttgat gtaccctagc tacaccttca gtggtgatgt tcagctagct caggatgaca |
| 901 | ttgatggcat ccaagccata tatggacgtt cccaaaatcc tgtccagccc atcggcccac |
| 961 | aaaccccaaa agcgtgtgac agtaagctaa cctttgatgc tataactacg attcggggag |
| 1021 | aagtgatgtt ctttaaagac agattctaca tgcgcacaaa tcccttctac ccggaagttg |
| 1081 | agctcaattt catttctgtt ttctggccac aactgccaaa tgggcttgaa gctgcttacg |
| 1141 | aatttgccga cagagatgaa gtccggtttt tcaaagggaa taagtactgg gctgttcagg |
| 1201 | gacagaatgt gctacacgga taccccaagg acatctacag ctcctttggc ttccctagaa |
| 1261 | ctgtgaagca tatcgatgct gctctttctg aggaaaacac tggaaaaacc tacttctttg |
| 1321 | ttgctaacaa atactggagg tatgatgaat ataaacgatc tatggatcca ggttatccca |
| 1381 | aaatgatagc acatgacttt cctggaattg gccacaaagt tgatgcagtt ttcatgaaag |
| 1441 | atggattttt ctatttcttt catggaacaa gacaatacaa atttgatcct aaaacgaaga |
| 1501 | gaattttgac tctccagaaa gctaatagct ggttcaactg caggaaaaat tgaacattac |
| 1561 | taatttgaat ggaaaacaca tggtgtgagt ccaaagaagg tgttttcctg aagaactgtc |
| 1621 | tattttctca gtcattttta acctctagag tcactgatac acagaatata atcttattta |
| 1681 | tacctcagtt tgcatatttt tttactattt agaatgtagc cctttttgta ctgatataat |
| 1741 | ttagttccac aaatggtggg tacaaaaagt caagtttgtg gcttatggat tcatataggc |
| 1801 | cagagttgca aagatctttt ccagagtatg caactctgac gttgatccca gagagcagct |
| 1861 | tcagtgacaa acatatcctt tcaagacaga aagagacagg agacatgagt ctttgccgga |
| 1921 | ggaaaagcag ctcaagaaca catgtgcagt cactggtgtc accctggata ggcaagggat |
| 1981 | aactcttcta acacaaaata agtgttttat gtttggaata aagtcaacct tgtttctact |
| 2041 | gttttataca ctttcaaaaa aaaaaaaaaa aaaaaaaaaa a |

The amino acid sequence of human MMP1 is provided by GenBank Accession No. NP_002412.1 and is shown below (SEQ ID NO: 8). The signal peptide is underlined.

| (SEQ ID NO: 8) | |
|---|---|
| 1 | mhsfppllll lfwgvvshsf patletqeqd vdlvqkylek yynlkndqrq vekrrnsgpv |
| 61 | veklkqmqef fglkvtgkpd aetlkvmkqp rcgvpdvaqf vltegnprwe qthltyrien |
| 121 | ytpdlpradv dhaiekafql wsnvtpltft kvsegqadim isfvrgdhrd nspfdgpggn |
| 181 | lahafqpgpg iggdahfded erwtnnfrey nlhrvaahel ghslglshst digalmypsy |
| 241 | tfsqdvqlaq ddidgiqaiy qrsqnpvapi gpqtpkacds kltfdaitti rgevmffkdr |
| 301 | fymrtnpfyp evelnfisvf wpqlpnglea ayefadrdev rffkgnkywa vqgqnvlhgy |
| 361 | pkdiyssfgf prtvkhidaa lseentgkty ffvankywry deykrsmdpg ypkmiahdfp |
| 421 | gighkvdavf mkdgffyffh gtrqykfdpk tkriltlqka nswfncrkn |

The mRNA sequence of human vitronectin (VTN) is provided by GenBank Accession No. NM_000638.3 and is shown below (SEQ ID NO: 9).

| (SEQ ID NO: 9) | |
|---|---|
| 1 | gagcaaacag agcagcagaa aaggcagttc ctcttctcca gtgccctcct tccctgtctc |
| 61 | tgcctctccc tcccttcctc aggcatcaga gcggagactt cagggagacc agagcccagc |
| 121 | ttgccaggca ctgagctaga agccctgcca tggcacccct gagacccctt ctcatactgg |
| 181 | ccctgctggc atgggttgct ctggctgacc aagagtcatg caagggccgc tgcactgagg |
| 241 | gcttcaacgt ggacaagaag tgccagtgtg acgagctctg ctcttactac cagagctgct |
| 301 | gcacagacta tacggctgag tgcaagcccc aagtgactcg cggggatgtg ttcactatgc |
| 361 | eggaggatga gtacacggtc tatgacgatg gcgaggagaa aaacaatgcc actgtccatg |
| 421 | aacaggtggg gggcccctcc ctgacctctg acctccaggc ccagtccaaa gggaatcctg |
| 481 | agcagacacc tgttctgaaa cctgaggaag aggcccctgc gcctgaggtg ggcgcctcta |
| 541 | agcctgaggg gatagactca aggcctgaga cccttcatcc agggagacct cagcccccag |
| 601 | cagaggagga gctgtgcagt gggaagccct tcgacgcctt caccgacctc aagaacggtt |
| 661 | ccctctttgc cttccgaggg cagtactgct atgaactgga cgaaaaggca gtgaggcctg |
| 721 | ggtaccccaa gctcatccga gatgtctggg gcatcgaggg ccccatcgat gccgccttca |
| 781 | cccgcatcaa ctgtcagggg aagacctacc tcttcaaggg tagtcagtac tggcgctttg |
| 841 | aggatggtgt cctggaccct gattaccccc gaaatatctc tgacggcttc gatggcatcc |
| 901 | cggacaacgt ggatgcagcc ttggccctcc ctgcccatag ctacagtggc cgggagcggg |
| 961 | tctacttctt caaggggaaa cagtactggg agtaccagtt ccagcaccag cccagtcagg |
| 1021 | aggagtgtga aggcagctcc ctgtcggctg tgtttgaaca ctttgccatg atgcagcggg |
| 1081 | acagctggga ggacatcttc gagcttctct tctggggcag aacctctgct ggtaccagac |
| 1141 | agccccagtt cattagccgg gactggcacg gtgtgccagg gcaagtggac gcagccatgg |
| 1201 | ctggccgcat ctacatctca ggcatggcac cccgcccctc cttggccaag aaacaaaggt |
| 1261 | ttaggcatcg caaccgcaaa ggctaccgtt cacaacgagg ccacagccgt ggccgcaacc |
| 1321 | agaactcccg ccggccatcc cgcgccacgt ggctgtcctt gttctccagt gaggagagca |
| 1381 | acttgggagc caacaactat gatgactaca ggatggactg gcttgtgcct gccacctgtg |
| 1441 | aacccatcca gagtgtcttc ttcttctctg gagacaagta ctaccgagtc aatcttcgca |
| 1501 | cacggcgagt ggacactgtg gaccctccct acccacgctc catcgctcag tactggctgg |
| 1561 | gctgcccagc tcctggccat ctgtaggagt cagagcccac atggccgggc cctctgtagc |
| 1621 | tccctcctcc catctccttc ccccagccca ataaaggtcc cttagccccg agtttaaa |

The amino acid sequence of human VTN is provided by GenBank Accession No. NP_000629.3 and is shown below (SEQ ID NO: 10). The predicted signal peptide is underlined.

| (SEQ ID NO: 10) | |
|---|---|
| 1 | maplrpllil allawvalad qesckgrcte gfnvdkkcqc delcsyyqsc ctdytaeckp |
| 61 | qvtrgdvftm pedeytvydd geeknnatvh eqvggpslts dlqaqskgnp eqtpvlkpee |
| 121 | eapapevgas kpegidsrpe tlhpgrpqpp aeeelcsgkp fdaftdlkng slfafrgqyc |
| 181 | yeldekavrp gypklirdvw giegpidaaf trincqgkty lfkgsqywrf edgvldpdyp |
| 241 | rnisdgfdgi pdnvdaalal pahsysgrer vyffkgkqyw eyqfqhqpsq eecegsslsa |
| 301 | vfehfammqr dswedifell fwgrtsagtr qpqfisrdwh gvpgqvdaam agriyisgma |
| 361 | prpslakkqr frhrnrkgyr sqrghsrgrn qnsrrpsrat wlslfssees nlgannyddy |
| 421 | rmdwlvpatc epiqsvfffs gdkyyrvnlr trrvdtvdpp yprsiaqywl gcpapghl |

The mRNA sequence of human COL4A1 is provided by GenBank Accession No. NM_001845.4 and is shown below (SEQ ID NO: 11). The start and stop codons are bolded and underlined.

| (SEQ ID NO: 11) | |
|---|---|
| 1 | gcttggagcc gccgcacccg ggacggtgcg tagcgctgga agtccggcct tccgagagct |
| 61 | agctgtccgc cgcggccccc gcacgccggg cagccgtccc tcgccgcctc gggcgcgcca |
| 121 | ccatggggcc ccggctcagc gtctggctgc tgctgctgcc cgccgccctt ctgctccacg |
| 181 | aggagcacag ccgggccgct gcgaagggtg gctgtgctgg ctctggctgt ggcaaatgtg |
| 241 | actgccatgg agtgaaggga caaaagggtg aaagaggcct cccggggtta caaggtgtca |
| 301 | ttgggtttcc tggaatgcaa ggacctgagg ggccacaggg accaccagga caaaagggtg |
| 361 | atactggaga accaggacta cctggaacaa aagggacaag aggacctccg ggagcatctg |
| 421 | gctaccctgg aaacccagga cttcccggaa ttcctggcca agacggcccg ccaggccccc |
| 481 | caggtattcc aggatgcaat ggcacaaagg gggagagagg gccgctcggg cctcctggct |
| 541 | tgcctggttt cgctggaaat cccggaccac caggcttacc agggatgaag ggtgatccag |
| 601 | gtgagatact tggccatgtg cccgggatgc tgttgaaagg tgaaagagga tttcccggaa |
| 661 | tcccagggac tccaggccca ccaggactgc cagggcttca aggtcctgtt gggcctccag |
| 721 | gatttaccgg accaccaggt cccccaggcc ctcccggccc tccaggtgaa aagggacaaa |
| 781 | tgggcttaag ttttcaagga ccaaaaggtg acaagggtga ccaaggggtc agtgggcctc |
| 841 | caggagtacc aggacaagct caagttcaag aaaaaggaga cttcgccacc aagggagaaa |
| 901 | agggccaaaa aggtgaacct ggatttcagg ggatgccagg ggtcggagag aaaggtgaac |
| 961 | ccggaaaacc aggacccaga ggcaaacccg gaaaagatgg tgacaaaggg gaaaaaggga |
| 1021 | gtcccggttt tcctggtgaa cccgggtacc caggactcat aggccgccag ggcccgcagg |
| 1081 | gagaaaaggg tgaagcaggt cctcctggcc cacctggaat tgttataggc acaggacctt |
| 1141 | tgggagaaaa aggagagagg ggctaccctg gaactccggg gccaagagga gagccaggcc |
| 1201 | caaaaggttt cccaggacta ccaggccaac ccggacctcc aggcctccct gtacctgggc |
| 1261 | aggctggtgc ccctggcttc cctggtgaaa gaggagaaaa aggtgaccga ggatttcctg |
| 1321 | gtacatctct gccaggacca agtggaagag atgggctccc gggtcctcct ggttcccctg |
| 1381 | ggccccctgg gcagcctggc tacacaaatg gaattgtgga atgtcagccc ggacctccag |
| 1441 | gtgaccaggg tcctcctgga attccagggc agccaggatt tataggcgaa attggagaga |
| 1501 | aaggtcaaaa aggagagagt tgcctcatct gtgatataga cggatatcgg gggcctcccg |
| 1561 | ggccacaggg acccccggga gaaataggtt tcccagggca gccaggggcc aagggcgaca |
| 1621 | gaggtttgcc tggcagagat ggtgttgcag gagtgccagg ccctcaaggt acaccagggc |
| 1681 | tgataggcca gccaggagcc aagggggagc ctggtgagtt ttatttcgac ttgcggctca |
| 1741 | aaggtgacaa aggagaccca ggctttccag gacagcccgg catgacaggg agagcgggtt |
| 1801 | ctcctggaag agatggccat ccgggtcttc ctggccccaa gggctcgccg ggttctgtag |
| 1861 | gattgaaagg agagcgtggc ccccctggag gagttggatt cccaggcagt cgtggtgaca |
| 1921 | ccggcccccc tgggcctcca ggatatggtc ctgctggtcc cattggtgac aaaggacaag |
| 1981 | caggctttcc tggaggccct ggatccccag gcctgccagg tccaaagggt gaaccaggaa |
| 2041 | aaattgttcc tttaccaggc ccccctggag cagaaggact gccggggtcc ccaggcttcc |
| 2101 | caggtcccca aggagaccga ggctttcccg gaaccccagg aaggccaggc ctgccaggag |
| 2161 | agaagggcgc tgtgggccag ccaggcattg gatttccagg gccccccggc cccaaaggtg |
| 2221 | ttgacggctt acctggagac atggggccac cggggactcc aggtcgcccg ggatttaatg |
| 2281 | gcttacctgg gaacccaggt gtgcagggcc agaagggaga gcctggagtt ggtctaccgg |
| 2341 | gactcaaagg tttgccaggt cttcccggca ttcctggcac acccggggag aaggggagca |
| 2401 | ttggggtacc aggcgttcct ggagaacatg gagcgatcgg accccctggg cttcagggga |
| 2461 | tcagaggtga accgggacct cctggattgc caggctccgt ggggtctcca ggagttccag |
| 2521 | gaataggccc ccctggagct aggggtcccc ctggaggaca gggaccaccg gggttgtcag |
| 2581 | gccctcctgg aataaaagga gagaagggtt tccccggatt ccctggactg gacatgccgg |
| 2641 | gccctaaagg agataaaggg gctcaaggac tccctggcat aacgggacag tcggggctcc |
| 2701 | ctggccttcc tggacagcag ggggctcctg ggattcctgg gtttccaggt tccaagggag |
| 2761 | aaatgggcgt catggggacc cccgggcagc cgggctcacc aggaccagtg ggtgctcctg |
| 2821 | gattaccggg tgaaaaaggg gaccatggct ttccgggctc ctcaggaccc aggggagacc |
| 2881 | ctggcttgaa aggtgataag ggggatgtcg gtctccctgg caagcctggc tccatggata |
| 2941 | aggtggacat gggcagcatg aagggccaga aaggagacca aggagagaaa ggacaaattg |
| 3001 | gaccaattgg tgagaaggga tcccgaggag accctgggac cccaggagtg cctggaaagg |
| 3061 | acgggcaggc aggacagcct gggcagccag gacctaaagg tgatccaggt ataagtggaa |
| 3121 | ccccaggtgc tccaggactt ccgggaccaa aaggatctgt tggtggaatg ggcttgccag |
| 3181 | gaacacctgg agagaaaggt gtgcctggca tccctggccc acaaggttca cctggcttac |
| 3241 | ctggagacaa aggtgcaaaa ggagagaaag ggcaggcagg cccacctggc ataggcatcc |
| 3301 | cagggctgcg aggtgaaaag ggagatcaag ggatagcggg tttcccagga agccctggag |
| 3361 | agaagggaga aaaaggaagc attgggatcc caggaatgcc agggtcccca ggccttaaag |
| 3421 | ggtctcccgg gagtgttggc tatccaggaa gtcctgggct acctggagaa aaaggtgaca |
| 3481 | aaggcctccc aggattggat ggcatccctg gtgtcaaagg agaagcaggt cttcctggga |
| 3541 | ctcctggccc cacaggccca gctggccaga aaggggagcc aggcagtgat ggaatcccgg |
| 3601 | ggtcagcagg agagaagggt gaaccaggtc taccaggaag aggattccca gggtttccag |
| 3661 | gggccaaagg agacaaaggt tcaaagggtg aggtgggttt cccaggatta gccgggagcc |
| 3721 | caggaattcc tggatccaaa ggagagcaag gattcatggg tcctccgggg ccccagggac |
| 3781 | agccggggtt accgggatcc ccaggccatg ccacggaggg gcccaaagga gaccgcggac |
| 3841 | ctcagggcca gcctggcctg ccaggacttc cgggacccat ggggcctcca gggcttcctg |
| 3901 | ggattgatgg agttaaaggt gacaaaggaa atccaggctg gccaggagca cccggtgtcc |
| 3961 | cagggcccaa gggagaccct ggattccagg gcatgcctgg tattggtggc tctccaggaa |
| 4021 | tcacaggctc taagggtgat atggggcctc caggagttcc aggatttcaa ggtccaaaag |
| 4081 | gtcttcctgg cctccaggga attaaaggtg atcaaggcga tcaaggcgtc ccgggagcta |
| 4141 | aaggtctccc gggtcctcct ggccccccag gtccttacga catcatcaaa ggggagcccg |
| 4201 | ggctccctgg tcctgagggc cccccagggc tgaaagggct tcagggactg ccaggcccga |
| 4261 | aaggccagca aggtgttaca ggattggtgg gtatacctgg acctccaggt attcctgggt |
| 4321 | ttgacggtgc ccctggccag aaaggagaga tgggacctgc cgggcctact ggtccaagag |
| 4381 | gatttccagg tccaccaggc cccgatgggt tgccaggatc catggggccc ccaggcaccc |
| 4441 | catctgttga tcacggcttc cttgtgacca ggcatagtca aacaatagat gacccacagt |
| 4501 | gtccttctgg gaccaaaatt ctttaccacg ggtactcttt gctctacgtg caaggcaatg |
| 4561 | aacgggccca tggccaggac ttgggcacgg ccggcagctg cctgcgcaag ttcagcacaa |
| 4621 | tgcccttcct gttctgcaat attaacaacg tgtgcaactt tgcatcacga aatgactact |
| 4681 | cgtactggct gtccacccct gagcccatgc ccatgtcaat ggcacccatc acgggggaaa |
| 4741 | acataagacc atttattagt aggtgtgctg tgtgtgaggc gcctgccatg gtgatggccg |
| 4801 | tgcacagcca gaccattcag atcccaccgt gccccagcgg gtggtcctcg ctgtggatcg |
| 4861 | gctactcttt tgtgatgcac accagcgctg gtgcagaagg ctctggccaa gccctggcgt |
| 4921 | cccccggctc ctgcctggag gagtttagaa gtgcgccatt catcgagtgt cacggccgtg |
| 4981 | ggacctgcaa ttactacgca aacgcttaca gcttttggct cgccaccata gagaggagcg |
| 5041 | agatgttcaa gaagcctacg ccgtccacct tgaaggcagg ggagctgcgc acgcacgtca |
| 5101 | gccgctgcca agtctgtatg agaagaacat aatgaagcct gactcagcta atgtcacaac |
| 5161 | atggtgctac ttcttcttct ttttgttaac agcaacgaac cctagaaata tatcctgtgt |
| 5221 | acctcactgt ccaatatgaa aaccgtaaag tgccttatag gaatttgcgt aactaacaca |
| 5281 | ccctgcttca ttgacctcta cttgctgaag gagaaaaaga cagcgataag ctttcaatag |
| 5341 | tggcatacca aatggcactt ttgatgaaat aaaatatcaa tattttctgc aatccaatgc |
| 5401 | actgatgtgt gaagtgagaa ctccatcaga aaaccaaagg gtgctaggag gtgtgggtgc |
| 5461 | cttccatact gtttgcccat tttcattctt gtattataat taattttcta cccccagaga |
| 5521 | taaatgtttg tttatatcac tgtctagctg tttcaaaatt taggtccctt ggtctgtaca |
| 5581 | aataatagca atgtaaaaat ggttttttga acctccaaat ggaattacag actcagtagc |
| 5641 | catatcttcc aaccccccag tataaatttc tgtctttctg ctatgtgtgg tactttgcag |
| 5701 | ctgcttttgc agaaatcaca attttcctgt ggaataaaga tggtccaaaa atagtcaaaa |
| 5761 | attaaatata tatatatatt agtaatttat atagatgtca gcaattaggc agatcaaggt |
| 5821 | ttagtttaac ttccactgtt aaaataaagc ttacatagtt ttcttccttt gaaagactgt |
| 5881 | gctgtccttt aacataggtt tttaaagact aggatattga atgtgaaaca tccgttttca |
| 5941 | ttgttcactt ctaaaccaaa aattatgtgt tgccaaaacc aaacccaggt tcatgaatat |
| 6001 | ggtgtctatt atagtgaaac atgtactttg agcttattgt ttttattctg tattaaatat |
| 6061 | tttcagggtt ttaaacacta atcacaaact gaatgacttg acttcaaaag caacaacctt |
| 6121 | aaaggccgtc atttcattag tattcctcat tctgcatcct ggcttgaaaa acagctctgt |
| 6181 | tgaatcacag tatcagtatt ttcacacgta agcacattcg ggccatttcc gtggtttctc |
| 6241 | atgagctgtg ttcacagacc tcagcagggc atcgcatgga ccgcaggagg gcagattcgg |
| 6301 | accactaggc ctgaaatgac atttcactaa aagtctccaa aacatttcta agactactaa |
| 6361 | ggccttttat gtaatttctt taaatgtgta tttcttaaga attcaaattt gtaataaaac |
| 6421 | tatttgtata aaaattaagc ttttattaat ttgttgctag tattgccaca gacgcattaa |
| 6481 | aagaaactta ctgcacaagc tgctaataaa tttgtaagct ttgcatacct taaaaaaaaa |
| 6541 | aaaaaaaaa |

The amino acid sequence of human COL4A1 is provided by GenBank Accession No. NP_001836.2 and is shown below (SEQ ID NO: 12). The signal peptide is underlined.

| (SEQ ID NO: 12) | |
|---|---|
| 1 | mgprlsvwll llpaalllhe ehsraaakgg cagsgcgkcd chgvkgqkge rglpglqgvi |
| 61 | gfpgmqgpeg pqgppgqkgd tgepglpgtk gtrgppgasg ypgnpglpgi pgqdgppgpp |
| 121 | gipgengtkg ergplgppgl pgfagnpgpp glpgmkgdpg eilghvpgml lkgergfpgi |
| 181 | pgtpgppglp glqgpvgppg ftgppgppgp pgppgekgqm glsfqgpkgd kgdqgvsgpp |
| 241 | gvpgqaqvqe kgdfatkgek gqkgepgfqg mpgvgekgep gkpgprgkpg kdgdkgekgs |
| 301 | pgfpgepgyp gligrqgpqg ekgeagppgp pgivigtgpl gekgergypg tpgprgepgp |
| 361 | kgfpglpgqp gppglpvpgq agapgfpger gekgdrgfpg tslpgpsgrd glpgppgspg |
| 421 | ppgqpgytng ivecqpgppg dqgppgipgq pgfigeigek gqkgesclic didgyrgppg |
| 481 | pqgppgeigf pgqpgakgdr glpgrdgvag vpgpqgtpgl igqpgakgep gefyfdlrlk |
| 541 | gdkgdpgfpg qpgmtgrags pgrdghpglp gpkgspgsvg lkgergppgg vgfpgsrgdt |
| 601 | gppgppgygp agpigdkgqa gfpggpgspg lpgpkgepgk ivplpgppga eglpgspgfp |
| 661 | gpqgdrgfpg tpgrpglpge kgavgqpgig fpgppgpkgv dglpgdmgpp gtpgrpgfng |
| 721 | lpgnpgvqgq kgepgvglpg lkglpglpgi pgtpgekgsi gvpgvpgehg aigppglqgi |
| 781 | rgepgppglp gsvgspgvpg igppgargpp ggqgppglsg ppgikgekgf pgfpgldmpg |
| 841 | pkgdkgaqgl pgitgqsglp glpgqqgapg ipgfpgskge mgvmgtpgqp gspgpvgapg |
| 901 | lpgekgdhgf pgssgprgdp glkgdkgdvg lpgkpgsmdk vdmgsmkgqk gdqgekgqig |
| 961 | pigekgsrgd pgtpgvpgkd gqagqpgqpg pkgdpgisgt pgapglpgpk gsvggmglpg |
| 1021 | tpgekgvpgi pgpqgspglp gdkgakgekg qagppgigip glrgekgdqg iagfpgspge |
| 1081 | kgekgsigip gmpgspglkg spgsvgypgs pglpgekgdk glpgldgipg vkgeaglpgt |
| 1141 | pgptgpagqk gepgsdgipg sagekgepgl pgrgfpgfpg akgdkgskge vgfpglagsp |
| 1201 | gipgskgeqg fmgppgpqgq pglpgspgha tegpkgdrgp qgqpglpglp gpmgppglpg |
| 1261 | idgvkgdkgn pgwpgapgvp gpkgdpgfqg mpgiggspgi tgskgdmgpp gvpgfqgpkg |
| 1321 | lpglqgikgd qgdqgvpgak glpgppgppg pydiikgepg lpgpegppgl kglqglpgpk |
| 1381 | gqqgvtglvg ipgppgipgf dgapgqkgem gpagptgprg fpgppgpdgl pgsmgppgtp |
| 1441 | svdhgflvtr hsqtiddpqc psgtkilyhg ysllyvqgne rahgqdlgta gsclrkfstm |
| 1501 | pflfcninnv cnfasrndys ywlstpepmp msmapitgen irpfisrcav ceapamvmav |
| 1561 | hsqtiqippc psgwsslwig ysfvmhtsag aegsgqalas pgscleefrs apfiechgrg |
| 1621 | tcnyyanays fwlatierse mfkkptpstl kagelrthvs rcqvemrrt |

The mRNA sequence of human COL4A3 is provided by GenBank Accession No. NM_000091.4 and is shown below (SEQ ID NO: 13). The start and stop codons are bolded and underlined.

| (SEQ ID NO: 13) | |
|---|---|
| 1 | gggagggacg aaccgcgcga ccgagcccta caaaacccgc cccggccgag tggcgaggcg |
| 61 | agctttccag ccgggctccc agagccgcgc tgcgcaggag acgcggtggc ctgagagcct |
| 121 | gagggtcccc ggactcgccc aggctctgag cgcgcgccca ccatgagcgc ccggaccgcc |
| 181 | cccaggccgc aggtgctcct gctgccgctc ctgctggtgc tcctggcggc ggcgcccgca |
| 241 | gccagcaagg gttgtgtctg taaagacaaa ggccagtgct tctgtgacgg ggccaaaggg |
| 301 | gagaaggggg agaagggctt tcctggaccc cccggttctc ctggccagaa aggattcaca |
| 361 | ggtcctgaag gcttgcctgg accgcaggga cccaagggct ttccaggact tccaggactc |
| 421 | acgggttcca aaggtgtaag gggaataagt ggattgccag gattttctgg ttctcctgga |
| 481 | cttccaggca ccccaggcaa taccgggcct tacggacttg tcggtgtacc aggatgcagt |
| 541 | ggttctaagg gtgagcaggg gtttccagga ctcccaggga cactgggcta cccagggatc |
| 601 | ccgggtgctg ctggtttgaa aggacaaaag ggtgctcctg ctaaagaaga agatatagaa |
| 661 | cttgatgcaa aaggcgaccc cgggttgcca ggggctccag gaccccaggg tttgccaggc |
| 721 | cctccaggtt ttcctgggcc tgttggccca cctggtcctc cgggattctt tggctttcca |
| 781 | ggagccatgg gacctagagg acctaagggt cacatgggtg aaagagtgat aggacataaa |
| 841 | ggagagcggg gtgtgaaagg gttaacagga cccccgggac caccaggaac agttattgtg |
| 901 | accctaactg gcccagataa cagaacggac ctcaaggggg aaaagggaga caagggagca |
| 961 | atgggcgagc ctggacctcc tggaccctca ggactgcctg gagaatcata tggatctgaa |
| 1021 | aagggtgctc ctggagaccc tggcctgcag ggaaaacccg gaaaagatgg tgttcctggc |
| 1081 | ttccctggaa gtgagggagt caagggcaac aggggtttcc ctgggttaat gggtgaagat |
| 1141 | ggcattaagg gacagaaagg ggacattggc cctccaggat ttcgtggtcc aacagaatat |
| 1201 | tatgacacat accaggaaaa gggagatgaa ggcactccag gcccaccagg gcccagagga |
| 1261 | gctcgtggcc cacaaggtcc cagtggtccc cccggagttc ctggaagtcc tggatcatca |
| 1321 | aggcctggcc tcagaggagc ccctggatgg ccaggcctga aaggaagtaa aggggaacga |
| 1381 | ggccgcccag gaaaggatgc catggggact cctgggtccc caggttgtgc tggttcacca |
| 1441 | ggtcttccag gatcaccggg acctccagga ccgccaggtg acatcgtttt tcgcaagggt |
| 1501 | ccacctggag atcacggact gccaggctat ctagggtctc caggaatccc aggagttgat |
| 1561 | gggcccaaag gagaaccagg cctcctgtgt acacagtgcc cttatatccc agggcctccc |
| 1621 | ggtctcccag gattgccagg gttacatggt gtaaaaggaa tcccaggaag acaaggcgca |
| 1681 | gctggcttga aaggaagccc agggtcccca ggaaatacag gtcttccagg atttccaggt |
| 1741 | ttcccaggtg cccagggtga cccaggactt aaaggagaaa aaggtgaaac acttcagcct |
| 1801 | gaggggcaag tgggtgtccc aggtgacccg gggctcagag gccaacctgg gagaaagggc |
| 1861 | ttggatggaa ttcctggaac tccgggagtg aaaggattac caggacctaa aggcgaactg |
| 1921 | gctctgagtg gtgagaaagg ggaccaaggt cctccagggg atcctggctc ccctgggtcc |
| 1981 | ccaggacctg caggaccagc tggaccacct ggctacggac cccaaggaga acctggtctc |
| 2041 | cagggcacgc aaggagttcc tggagccccc ggaccacccg gagaagccgg ccctagggga |
| 2101 | gagctcagtg tttcaacacc agttccaggc ccaccaggac ctccagggcc ccctggccat |
| 2161 | cctggccccc aaggtccacc tggtatccct ggatccctgg ggaaatgtgg agatcctggt |
| 2221 | cttccagggc ctgatggtga accaggaatt ccaggaattg gatttcctgg gcctcctgga |
| 2281 | cctaagggag accaaggttt tccaggtaca aaaggatcac tgggttgtcc tggaaaaatg |
| 2341 | ggagagcctg ggttacctgg aaagccaggc ctcccaggag ccaagggaga accagcagta |
| 2401 | gccatgcctg gaggaccagg aacaccaggt tttccaggag aaagaggcaa ttctggggaa |
| 2461 | catggagaaa ttggactccc tggacttcca ggtctccctg gaactccagg aaatgaaggg |
| 2521 | cttgatggac cacgaggaga tccagggcag cctggaccac ctggagaaca aggaccccca |
| 2581 | ggaaggtgca tagagggtcc caggggagcc caaggacttc caggcttaaa tggattgaaa |
| 2641 | gggcaacaag gcagaagagg taaaacgggg ccaaagggag acccaggaat tccaggcttg |
| 2701 | gatagatcag gatttcctgg agaaactgga tcaccaggaa ttccaggtca tcaaggtgaa |
| 2761 | atgggaccac tgggtcaaag aggatatcca ggaaatccgg gaattttagg gccaccaggt |
| 2821 | gaagatggag tgattgggat gatgggcttt cctggagcca ttggccctcc agggccccct |
| 2881 | gggaacccag gcacaccagg gcagaggggg agccctggaa ttccaggagt aaagggccag |
| 2941 | agaggaaccc caggagccaa gggggaacaa ggagataaag gaaatcccgg gccttcagag |
| 3001 | atatcccacg taatagggga caaaggagaa ccaggtctca aaggattcgc aggaaatcca |
| 3061 | ggtgagaaag gaaacagagg cgttccaggg atgccaggtt taaagggcct caaaggacta |
| 3121 | cccggaccag caggaccacc aggccccaga ggagatttgg gcagcactgg gaatcctgga |
| 3181 | gaaccaggac tgcgtggtat accaggaagc atggggaaca tgggcatgcc aggttctaaa |
| 3241 | ggaaaaaggg gaactttggg attcccaggt cgagcaggaa gaccaggcct cccaggtatt |
| 3301 | catggtctcc agggagataa gggagagcca ggttattcag aaggtacaag gccaggacca |
| 3361 | ccgggaccaa cgggggatcc aggactgccg ggtgatatgg gaaagaaagg agaaatgggg |
| 3421 | caacctggcc cacctggaca tttggggcct gctggacctg agggagcccc tggaagtcct |
| 3481 | ggaagtcctg gcctcccagg aaagccaggt cctcatggtg atttgggttt taaaggaatc |
| 3541 | aaaggcctcc tgggccctcc aggaatcaga ggccctccag gtcttccagg atttccagga |
| 3601 | tctcctggac caatgggtat aagaggtgac caaggacgtg atggaattcc tggtccagcc |
| 3661 | ggagaaaagg gagaaacggg tttattgagg gcccctccag gcccaagagg gaaccctggt |
| 3721 | gctcaaggag ccaaaggaga caggggagcc ccaggttttc ctggcctccc gggcagaaaa |
| 3781 | ggggccatgg gagatgctgg acctcgagga cccacaggca tagaaggatt cccagggcca |
| 3841 | ccaggtctgc ccggtgcaat tatccctggc cagacaggaa atcgtggtcc accaggctca |
| 3901 | agaggaagcc caggtgcgcc tggtccccct ggacctccag ggagtcatgt aataggcata |
| 3961 | aaaggagaca aagggtctat gggccaccct ggcccaaaag gtccacctgg aactgcagga |
| 4021 | gacatgggac caccaggtcg tctgggagca ccaggtactc caggtcttcc aggacccaga |
| 4081 | ggtgatcctg gattccaggg gtttccaggc gtgaaaggag aaaagggtaa tcctggattt |
| 4141 | ctaggatcca ttggacctcc aggaccaatt gggccaaaag gaccacctgg tgtacgtgga |
| 4201 | gaccctggca cacttaagat tatctccctt ccaggaagcc cagggccacc tggcacacct |
| 4261 | ggagaaccag ggatgcaggg agaacctggg ccaccagggc cacctggaaa cctaggaccc |
| 4321 | tgtgggccaa gaggtaagcc aggcaaggat ggaaaaccag gaactcctgg accagctgga |
| 4381 | gaaaaaggca acaaaggttc taaaggagag ccaggaccag ctggatcaga tggattgcca |
| 4441 | ggtttgaaag gaaaacgtgg agacagtgga tcacctgcaa cctggacaac gagaggcttt |
| 4501 | gtcttcaccc gacacagtca aaccacagca attccttcat gtccagaggg gacagtgcca |
| 4561 | ctctacagtg ggttttcttt tctttttgta caaggaaatc aacgagccca cggacaagac |
| 4621 | cttggaactc ttggcagctg cctgcagcga tttaccacaa tgccattctt attctgcaat |
| 4681 | gtcaatgatg tatgtaattt tgcatctcga aatgattatt catactggct gtcaacacca |
| 4741 | gctctgatgc caatgaacat ggctcccatt actggcagag cccttgagcc ttatataagc |
| 4801 | agatgcactg tttgtgaagg tcctgcgatc gccatagccg ttcacagcca aaccactgac |
| 4861 | attcctccat gtcctcacgg ctggatttct ctctggaaag gattttcatt catcatgttc |
| 4921 | acaagtgcag gttctgaggg caccgggcaa gcactggcct cccctggctc ctgcctggaa |
| 4981 | gaattccgag ccagcccatt tctagaatgt catggaagag gaacgtgcaa ctactattca |
| 5041 | aattcctaca gtttctggct ggcttcatta aacccagaaa gaatgttcag aaagcctatt |
| 5101 | ccatcaactg tgaaagctgg ggaattagaa aaaataataa gtcgctgtca ggtgtgcatg |
| 5161 | aagaaaagac actgaagcta aaaaagacag cagaactgct atttttcatc ctaaagaaca |
| 5221 | aagtaatgac agaacatgct gttatttagg tatttttctt taaccaaaca atattgctcc |
| 5281 | atgatgactt agtacaaagt ttcaatttgt ttccccacaa aacaaagcaa ttctttcaag |
| 5341 | tcagttctgt gatctgggtc tctaatctgt gctgtttcaa agttctctgt ggcaaagcag |
| 5401 | caactattca caaaatatca ccaaaaacct attccactta catccaaggc actgtcacta |
| 5461 | cggtgattgt atgaagtttg aatgctgcaa gttatgaaat atttggcccg ctggattccc |
| 5521 | acatttgtct tctttctgtc tttaagactc agggaggeta aatcagtgtt tgattgcccc |
| 5581 | gccaaccctt cctgaaactt cagaccctgg gtaggggaag agaagggggc atgtggtatc |
| 5641 | ctggagcatt gtgtatagaa ctggattttc agacctgctg aggaccgtaa ggcctgatgg |
| 5701 | aacacagaac tgaactgagg ttcatggatt ttccaggact gtttcaaaca tgcccattac |
| 5761 | taacggcaaa agggggattc cctgatggaa ccataatacc ettggaaata ctgtatggtt |
| 5821 | ttgttttgtt ttgttggttt ttaaagattt ttgtttgttt attgaattca tttcactgta |
| 5881 | gctctaaaat ctgcttgtat tccaagcata taaaattttc ccccttagtg aattagtttt |
| 5941 | aaaatgatat tgttatatac atactatgaa atatgtataa ctttaacttc tgttttacca |
| 6001 | gcatacccac acaaataaca agaatactac ttatgaaatg tgcactttat cctcattcca |
| 6061 | taaatgtcgg tgcatacctt atgtaaggga gcagttcaat aatccatgaa agaacttaag |
| 6121 | gcatttgttg gtttatcaga ctcggaatct attttctcat tgctctgaat atgtcatcac |
| 6181 | tctaggtttt acagatttat tcctttgtta cttctctaat tcttcctttg taaaaaaaaa |
| 6241 | aaaaagcaac actttttatg ttatatgttg ttcttacaaa ccatactgaa agagtccatt |
| 6301 | gtttaaaaat cttaatgtat caaactgtat aacttggccg ctgtatgtct taaaacctgc |
| 6361 | ttttcaatgt gttgatacat tcccaaggtt acttaattca acttaactat catcttattc |
| 6421 | agcaccaagc atgtcccagg cactgtacta acctacagag atgctaagag aaaaaaaaga |
| 6481 | cttgtttctg atctaatatc ccagaaaaag taactcattg ctctgttaat aatctcacat |
| 6541 | atacaagtag cttccctccc ctctagtttt ttcttccttt tcactgctgt tatatttcat |
| 6601 | catgataatt cagcaggccc aagtaaaggt taaaaataag gtctatgcct agggaaactc |
| 6661 | agggcttcta gtttctctta gaaaagctaa gagaagataa ggtctgaata atagcagaaa |
| 6721 | aaccaacatc tacaaaacat taaactagtg ttatacttga tgataacact atttgatgag |
| 6781 | tcttagagtc cagacacaaa gagacaaagc tttgaagatg ctttttgatc tacctaggtg |
| 6841 | gagttggtgg tgctgatatt taaattcagg ctactgcttc aatctcaatt gctttgtaag |
| 6901 | tgaaaaacat gacccagagg acagcacaga ctatggccat ggctcacatg gtttacatcc |
| 6961 | ttcactgctc acgtgtttgc tgtcaagcca tttttacatc taaactaaga tgtgcagcat |
| 7021 | ttcacttatt tagattcact taacaaacaa atttttctgc tttaaaaatg tcttattgtc |
| 7081 | ccaagtgtac tatagcggca tatagagcta gctaatctct acaaaccctc tgtaggccag |
| 7141 | tagttctcaa agtgtggtct ctggaagagc agtatcagca tcatctggga acttgtcaca |
| 7201 | gatgeagatt ctagggacca ctccagacct acacaatcag aaactcttgg gggagggccc |
| 7261 | gaaatatcta tgttttacca agcccaccac atgattctga tgtactctaa atactgagaa |
| 7321 | aacctgttct agacaaatac ccaagcaaca actccgcagg cagttaccaa gtacggctgg |
| 7381 | ctacaactgc tccatccgtg cctcttttta aagttcaaac tcacaggtga ctctaaggtt |
| 7441 | atctactttt actcataagt aaaagcccta gactggtgct aatgtcaaac cactggcctc |
| 7501 | cactcaggcc tccatcttct catgccctct taccagtatt taacttctga ggaagacaag |
| 7561 | tgatgctaaa acctgaaatt ccaatgaagc catatgaaca gctgttcagt tgcacttcta |
| 7621 | agactttact tagcagtaaa ttatagctca tgtgcattat tttccagata acttagctta |
| 7681 | tgagtagctt atacaattat gaagatttaa tattacagat aaaatgtaaa ctgtttcttt |
| 7741 | aaaattgggg cttcaacttt ggaatttcac agcgtgctaa aataacagat ttctcagaag |
| 7801 | tctttcagca agataaacat tattaagtaa cttatttatg aaagtattaa aatgcttaca |
| 7861 | tttgaacttg atggctaact tacaaagatt ctctatgtat caaatgtaac ttactgcgac |
| 7921 | taaacttaat ttaatattta ctctataacc aaatgaaata tatttaaaat atattgaata |
| 7981 | ttttatattg ttatatcctg acaagattat aatattttaa tgtactaata tttctgtaat |
| 8041 | tatatctaaa atattatttt attatattgc ctaagaataa acatttgtta aattggaaaa |
| 8101 | aaaaaaaaaa aaaa |

The protein sequence of human COL4A3 is provided by GenBank Accession No. NP_000082.2 and is shown below (SEQ ID NO: 14). The predicted signal peptide is underlined.

| (SEQ ID NO: 14) | |
|---|---|
| 1 | msartaprpq vlllplllvl laaapaaskg cvckdkgqcf cdgakgekge kgfpgppgsp |
| 61 | gqkgftgpeg ipgpqgpkgf pglpgltgsk gvrgisglpg fsgspglpgt pgntgpyglv |
| 121 | gvpgcsgskg eqgfpglpgt Igypgipgaa glkgqkgapa keedieldak gdpglpgapg |
| 181 | pqglpgppgf pgpvgppgpp gffgfpgamg prgpkghmge rvighkgerg vkgltgppgp |
| 241 | pgtvivtltg pdnrtdlkge kgdkgamgep gppgpsgipg esygsekgap gdpglqgkpg |
| 301 | kdgvpgfpgs egvkgnrgfp glmgedgikg qkgdigppgf rgpteyydty qekgdegtpg |
| 361 | ppgprgargp qgpsgppgvp gspgssrpgl rgapgwpglk gskgergrpg kdamgtpgsp |
| 421 | gcagspglpg spgppgppgd ivfrkgppgd hglpgylgsp gipgvdgpkg epgllctqcp |
| 481 | yipgppglpg lpglhgvkgi pgrqgaaglk gspgspgntg ipgfpgfpga qgdpglkgek |
| 541 | getlqpegqv gvpgdpglrg qpgrkgldgi pgtpgvkglp gpkgelalsg ekgdqgppgd |
| 601 | pgspgspgpa gpagppgygp qgepglqgtq gvpgapgppg eagprgelsv stpvpgppgp |
| 661 | pgppghpgpq gppgipgslg kcgdpglpgp dgepgipgig fpgppgpkgd qgfpgtkgsl |
| 721 | gcpgkmgepg lpgkpglpga kgepavampg gpgtpgfpge rgnsgehgei glpglpglpg |
| 781 | tpgnegldgp rgdpgqpgpp geqgppgrci egprgaqglp glnglkgqqg rrgktgpkgd |
| 841 | pgipgldrsg fpgetgspgi pghqgemgpl gqrgypgnpg ilgppgedgv igmmgfpgai |
| 901 | gppgppgnpg tpgqrgspgi pgvkgqrgtp gakgeqgdkg npgpseishv igdkgepglk |
| 961 | gfagnpgekg nrgvpgmpgl kglkglpgpa gppgprgdlg stgnpgepgl rgipgsmgnm |
| 1021 | gmpgskgkrg tigfpgragr pglpgihglq gdkgepgyse gtrpgppgpt gdpglpgdmg |
| 1081 | kkgemgqpgp pghlgpagpe gapgspgspg ipgkpgphgd lgfkgikgll gppgirgppg |
| 1141 | lpgfpgspgp mgirgdqgrd gipgpagekg etgllrappg prgnpgaqga kgdrgapgfp |
| 1201 | glpgrkgamg dagprgptgi egfpgppglp gaiipgqtgn rgppgsrgsp gapgppgppg |
| 1261 | shvigikgdk gsmghpgpkg ppgtagdmgp pgrigapgtp glpgprgdpg fqgfpgvkge |
| 1321 | kgnpgflgsi gppgpigpkg ppgvrgdpgt ikiislpgsp gppgtpgepg mqgepgppgp |
| 1381 | pgnigpcgpr gkpgkdgkpg tpgpagekgn kgskgepgpa gsdglpglkg krgdsgspat |
| 1441 | wttrgfvftr hsqttaipsc pegtvplysg fsfifvqgnq rahgqdlgtl gsclqrfttm |
| 1501 | pflfcnvndv cnfasrndys ywlstpalmp mnmapitgra lepyisrctv cegpaiaiav |
| 1561 | hsqttdippc phgwislwkg fsfimftsag segtgqalas pgscleefra spflechgrg |
| 1621 | tcnyysnsys fwlaslnper mfrkpipstv kagelekiis rcqvcmkkrh |

The mRNA sequence of human COL5A3 is provided by GenBank Accession No. NM_015719.3 and is shown below (SEQ ID NO: 15). The start and stop codons are bolded and underlined.

| (SEQ ID NO: 15) | |
|---|---|
| 1 | gcgagtgact gcaccgagcc cgagaagtcg ccgcgccccg cagccgcccc gactggttcc |
| 61 | ccgccttgcc cgtgggcccc gccgggatgg ggaaccgccg ggacctgggc cagccgcggg |
| 121 | ccggtctctg cctgctcctg gccgcgctgc agcttctgcc ggggacgcag gccgatcctg |
| 181 | tggatgtcct gaaggccctg ggtgtgcagg gaggccaggc tggggtcccc gaggggcctg |
| 241 | gcttctgtcc ccagaggact ccagagggtg accgggcatt cagaattggc caggccagca |
| 301 | cgctcggcat ccccacgtgg gaactctttc cagaaggcca ctttcctgag aacttctcct |
| 361 | tgctgatcac cttgcgggga cagccagcca atcagtctgt cctgctgtcc atttatgatg |
| 421 | aaaggggtgc ccggcagttg ggcctggcac tggggccagc gctgggtctc ctaggtgacc |
| 481 | ccttccgccc cctcccccag caggtcaacc tcacagatgg caggtggcac cgtgtggccg |
| 541 | tcagcataga tggtgagatg gtgaccctgg tagctgactg tgaagctcag ccccctgttt |
| 601 | tgggccatgg cccccgcttc atcagcatag ctggactcac tgtgctgggg acccaggacc |
| 661 | ttggggaaaa gactttcgag ggagacattc aggagctgct gataagccca gatcctcagg |
| 721 | ctgccttcca ggcttgtgag cggtacctcc ccgactgtga caacctggca ccggcagcca |
| 781 | cagtggctcc ccagggtgaa ccagaaaccc ctcgtcctcg gcggaagggg aagggaaaag |
| 841 | ggaggaagaa agggcgaggt cgcaagggga agggcaggaa aaagaacaag gaaatttgga |
| 901 | cctcaagtcc acctcctgac tccgcagaga accagacctc cactgacatc cccaagacag |
| 961 | agactccagc tccaaatctg cctccgaccc ccacgccttt ggtcgtcacc tccactgtga |
| 1021 | ctactggact caatgccacg atcctagaga ggagcttgga ccctgacagt ggaaccgagc |
| 1081 | tggggaccct ggagaccaag gcagccaggg aggatgaaga aggagatgat tccaccatgg |
| 1141 | gccctgactt ccgggcagca gaatatccat ctcggactca gttccagatc tttcctggtg |
| 1201 | ctggagagaa aggagcaaaa ggagagcccg cagtgattga aaaggggcag cagtttgagg |
| 1261 | gacctccagg agccccagga ccccaagggg tggttggccc ctcaggccct cccggccccc |
| 1321 | caggattccc tggcgaccct ggtccaccgg gccctgctgg cctcccagga atccccggca |
| 1381 | ttgatgggat ccgaggccca ccgggcactg tgatcatgat gccgttccag tttgcaggcg |
| 1441 | gctcctttaa aggcccccca gtctcattcc agcaggccca ggctcaggca gttctgcagc |
| 1501 | agactcagct ctctatgaaa ggcccccctg gtccagtggg gctcactggg cgcccaggcc |
| 1561 | ctgtgggtct ccccgggcat ccaggtctga aaggagagga gggagcagaa gggccacagg |
| 1621 | gtccccgagg cctgcaggga cctcatggac cccctggccg agtgggcaag atgggccgcc |
| 1681 | ctggagcaga tggagctcgg ggcctcccag gggacactgg acctaagggt gatcgtggct |
| 1741 | tcgatggcct ccctgggctg cctggtgaga agggccaaag gggtgacttt ggccatgtgg |
| 1801 | ggcaacccgg tcccccagga gaggatggtg agaggggagc agagggacct ccagggccca |
| 1861 | ctggccaggc tggggagccg ggtccacgag gactgcttgg ccccagaggc tctcctggcc |
| 1921 | ccacgggtcg cccgggtgtg actggaattg atggtgctcc tggtgccaaa ggcaatgtgg |
| 1981 | gtcctccagg agaaccaggc cctccgggac agcagggaaa ccatgggtcc cagggactcc |
| 2041 | ccggtcccca gggactcatt ggcactcctg gggagaaggg tccccctgga aacccaggaa |
| 2101 | ttccaggcct cccaggatcc gatggccctc tgggtcaccc aggacatgag ggccccacgg |
| 2161 | gagagaaagg ggctcagggt ccaccagggt cggcaggccc tccgggctat cctggacctc |
| 2221 | ggggagtgaa gggcacttca ggcaaccggg gcctccaggg ggagaaaggc gagaagggag |
| 2281 | aggacggctt cccaggcttc aagggcgatg tggggctcaa aggtgatcag gggaaacccg |
| 2341 | gagctccagg tccccgggga gaggatggtc ctgaggggcc gaaggggcag gcggggcagg |
| 2401 | ctggcgagga ggggccccca ggctcagctg gggagaaggg caagcttggg gtgccaggcc |
| 2461 | tcccaggtta tccaggacgc cctggaccta agggatctat tggatttccc ggtcccctgg |
| 2521 | gacccatagg agagaaaggg aagtcgggaa agacagggca gccaggcctg gaaggagagc |
| 2581 | ggggaccacc aggttcccgt ggagagaggg ggcaaccggg tgccacaggg caaccaggcc |
| 2641 | ccaagggcga tgtgggccag gatggagccc ctgggatccc tggagaaaag ggcctccctg |
| 2701 | gtctgcaagg ccctccagga ttccctgggc caaagggccc ccctggtcac caaggtaaag |
| 2761 | atgggcgacc agggcaccct ggacagagag gagaactggg cttccaaggt cagacaggcc |
| 2821 | cgcctggacc agctggtgtc ttaggccctc agggaaagac aggagaagtg ggacctctag |
| 2881 | gtgaaagggg gcctccaggc ccccctggac ctcctggtga acaaggtctt cctggcctgg |
| 2941 | aaggcagaga gggggccaag ggggaactgg gaccaccagg accccttggg aaagaagggc |
| 3001 | cagctggact caggggcttt cccggcccca aagggggccc tggggacccg ggacctactg |
| 3061 | gcttaaaggg tgataagggc cccccagggc ccgtgggggc caatggctcc cctggtgagc |
| 3121 | gcggtccttt gggcccagca ggaggcattg gacttcctgg ccaaagtggc agcgaaggcc |
| 3181 | ccgttggccc tgcaggcaag aaggggtccc ggggagaacg tggcccccct ggccccactg |
| 3241 | gcaaagatgg gatcccaggg cccctggggc ctctgggacc ccctggagct gctgggcctt |
| 3301 | ctggcgagga aggggacaag ggggatgtgg gtgcccccgg acacaagggg agtaaaggcg |
| 3361 | ataaaggaga cgcgggccca cctggacaac cagggatacg gggtcctgca ggacacccag |
| 3421 | gtcccccggg agcagacggg gctcaggggc gccggggacc cccaggcctc tttgggcaga |
| 3481 | aaggagatga cggagtcaga ggctttgtgg gggtgattgg ccctcctgga ctgcaggggc |
| 3541 | tgccaggccc tccgggagag aaaggggagg tcggagacgt cgggtccatg ggtccccatg |
| 3601 | gagctccagg tcctcggggt ccccaaggcc ccactggatc agagggcact ccagggctgc |
| 3661 | ctggaggagt tggtcagcca ggcgccgtgg gtgagaaggg tgagcgaggg gacgctggag |
| 3721 | acccagggcc tccaggagcc ccaggcatcc cggggcccaa gggagacatt ggtgaaaagg |
| 3781 | gggactcagg cccatctgga gctgctggac ccccaggcaa gaaaggtccc cctggagagg |
| 3841 | atggagccaa agggagcgtg ggccccacgg ggctgcccgg agatctaggg cccccaggag |
| 3901 | accctggagt ttcaggcata gatggttccc caggggagaa gggagaccct ggtgatgttg |
| 3961 | ggggaccggg tccgcctgga gcttctgggg agcccggcgc ccccgggccc cccggcaaga |
| 4021 | ggggtccttc aggccacatg ggtcgagaag gcagagaagg ggagaaaggt gccaaggggg |
| 4081 | agccaggtcc tgatgggccc ccagggagga cgggtccaat gggggctaga gggccccctg |
| 4141 | gacgtgtggg gcctgagggt cttcgaggga tccctggccc tgtgggtgaa ccaggcctcc |
| 4201 | tgggagcccc tggacagatg ggccctcctg gccccctggg gccctctggc ctcccagggc |
| 4261 | tgaagggaga cactggcccc aagggggaaa agggccacat tggattgatc ggtctcattg |
| 4321 | gccccccggg agaagctggt gagaaaggag atcaggggtt gccaggcgtg cagggacccc |
| 4381 | ctggtcccaa gggagaccct ggtccccctg gtcccattgg ctctctgggc caccctgggc |
| 4441 | ccccaggtgt ggcgggccct ctaggacaga aaggctcaaa agggtctccg gggtccatgg |
| 4501 | gcccccgtgg agacactgga cctgcaggcc caccaggccc cccgggtgcc cctgccgagc |
| 4561 | tgcatgggct gcgcaggcgc cggcgcttcg tcccagtccc gcttccagtc gtggagggcg |
| 4621 | gcctggagga ggtgctggcc tcgctcacat cgctgagctt ggagctggag cagctgcggc |
| 4681 | gtcctcccgg cactgcggag cgcccgggcc tcgtgtgcca cgagctgcac cgcaaccacc |
| 4741 | cgcacctgcc tgatggggaa tactggattg accccaacca gggctgcgcg cgggactcgt |
| 4801 | tcagggtttt ttgcaacttc acggcgggag gagagacctg cctctatccc gacaagaagt |
| 4861 | ttgagatcgt gaaattggcc tcctggtcca aggaaaagcc tggaggctgg tatagcacat |
| 4921 | tccgtcgagg gaagaagttc tcctacgtgg acgccgacgg gtccccagtg aatgtcgtgc |
| 4981 | agctgaactt cctgaaactg ctgagtgcca cagctcgcca gaacttcacc tactcctgcc |
| 5041 | agaatgcagc tgcctggctg gacgaagcca cgggtgacta cagccactcc gcccgcttcc |
| 5101 | ttggcaccaa tggagaggag ctgtctttca accagacgac agcagccact gtcagcgtcc |
| 5161 | cccaggatgg ctgccggctc cggaaaggac agacgaagac ccttttcgaa ttcagctctt |
| 5221 | ctcgagcggg atttctgccc ctgtgggatg tggcggccac tgactttggc cagacgaacc |
| 5281 | aaaagtttgg gtttgaactg ggccccgtct gcttcagcag ctgagagtgt ccggggtggg |
| 5341 | agggaccatg agggagcccc agaatggggt gcatttggtg ctgaggcttt gaagccaccg |
| 5401 | tatttttcgt tacctgtgac tatggagcca atgggatgtg acttcgctca tcacggacag |
| 5461 | tcattccttc tcctttccag ggtgctgggg gctggggttc cctggcccaa gggtccagcc |
| 5521 | tcctctcacc ccattccagg tggcatactg cagtctggct ctttctcccc tccctcccca |
| 5581 | cccaagcctc acctccccac cccttgaacc cccatgcaat gagcttctaa ctcagagctg |
| 5641 | atgaacaaaa gcccccccac ccccaatgcc tgcctcctca ctcctccgtc gctgcccttc |
| 5701 | acaccttttg gtgctacccc tccccagagt taagcactgg atgtctcctg atcccaggct |
| 5761 | gggaccccta cccccacccc ctttgatcct ttctacttcc acggtgaaag gactgaggtc |
| 5821 | ggactacaga gggaagaggg acttcccttg actgggttgt gtttcttttc ctgcctcagc |
| 5881 | ccagctctgc aaatcccctc cccctgcccc ccacctcccc aggctcacct tgccatgcca |
| 5941 | ggtggtttgg ggaccaagat gttggggggg tgaatcagga tcctaatggt gctgccctat |
| 6001 | ttatacctgg gtctgtatta aaagggaaag tcccccctgt tgtagatttc atctgcttcc |
| 6061 | tccttaggga aggctgggat atgatgagag attccagccc aagcctggcc ccccaccgcc |
| 6121 | aggccatagg gcataatttg catctcaaat ctgagaataa actgatgaac tgtgaaaaaa |
| 6181 | aaaaaaaaaa aa |

The protein sequence of human COL5A3 is provided by GenBank Accession No. NP_056534.2 and is shown below (SEQ ID NO: 16). The signal peptide is underlined. The mature peptide is bolded and italicized.

| (SEQ ID NO: 16) | |
|---|---|
| 1 | mgnrrdlgqp raglclllaa lqllpgtqa [symbol] [symbol] |
| 61 | [symbol] [symbol] [symbol] |
| 121 | [symbol] [symbol] [symbol] |
| 181 | [symbol] [symbol] [symbol] |
| 241 | [symbol] [symbol] [symbol] |
| 301 | [symbol] [symbol] [symbol] |
| 361 | [symbol] [symbol] [symbol] |
| 421 | [symbol] [symbol] [symbol] |
| 481 | [symbol] [symbol] [symbol] |
| 541 | [symbol] [symbol] [symbol] |
| 601 | [symbol] [symbol] [symbol] |
| 661 | [symbol] [symbol] [symbol] |
| 721 | [symbol] [symbol] [symbol] |
| 781 | [symbol] [symbol] [symbol] |
| 841 | [symbol] [symbol] [symbol] |
| 901 | [symbol] [symbol] [symbol] |
| 961 | [symbol] [symbol] [symbol] |
| 1021 | [symbol] [symbol] [symbol] |
| 1081 | [symbol] [symbol] [symbol] |
| 1141 | [symbol] [symbol] [symbol] |
| 1201 | [symbol] [symbol] [symbol] |
| 1261 | [symbol] [symbol] [symbol] |
| 1321 | [symbol] [symbol] [symbol] |
| 1381 | [symbol] [symbol] [symbol] |
| 1441 | [symbol] [symbol] [symbol] |
| 1501 | fvpvplpvve ggleevlasl tslsleleql rrppgtaerp glvchelhrn hphlpdgeyw |
| 1561 | idpnqgcard sfrvfcnfta ggetclypdk kfeivklasw skekpggwys tfrrgkkfsy |
| 1621 | vdadgspvnv vqlnflklls atarqnftys cqnaaawlde atgdyshsar flgtngeels |
| 1681 | fnqttaatvs vpqdgcrlrk gqtktlfefs ssragflplw dvaatdfgqt nqkfgfelgp |
| 1741 | vcfss |

The mRNA sequence of human hepatocyte growth factor (HGF) is provided by GenBank Accession No. M73239.1 and is shown below (SEQ ID NO: 17). The start and stop codons are bolded and underlined.

| (SEQ ID NO: 17) | |
|---|---|
| 1 | ccgaacagga ttctttcacc caggcatctc ctccagaggg atccgccagc ccgtccagca |
| 61 | gcaccatgtg ggtgaccaaa ctcctgccag ccctgctgct gcagcatgtc ctcctgcatc |
| 121 | tcctcctgct ccccatcgcc atcccctatg cagagggaca aaggaaaaga agaaatacaa |
| 181 | ttcatgaatt caaaaaatca gcaaagacta ccctaatcaa aatagatcca gcactgaaga |
| 241 | taaaaaccaa aaaagtgaat actgcagacc aatgtgctaa tagatgtact aggaataaag |
| 301 | gacttccatt cacttgcaag gcttttgttt ttgataaagc aagaaaacaa tgcctctggt |
| 361 | tccccttcaa tagcatgtca agtggagtga aaaaagaatt tggccatgaa tttgacctct |
| 421 | atgaaaacaa agactacatt agaaactgca tcattggtaa aggacgcagc tacaagggaa |
| 481 | cagtatctat cactaagagt ggcatcaaat gtcagccctg gagttccatg ataccacacg |
| 541 | aacacagctt tttgccttcg agctatcggg gtaaagacct acaggaaaac tactgtcgaa |
| 601 | atcctcgagg ggaagaaggg ggaccctggt gtttcacaag caatccagag gtacgctacg |
| 661 | aagtctgtga cattcctcag tgttcagaag ttgaatgcat gacctgcaat ggggagagtt |
| 721 | atcgaggtct catggatcat acagaatcag gcaagatttg tcagcgctgg gatcatcaga |
| 781 | caccacaccg gcacaaattc ttgcctgaaa gatatcccga caagggcttt gatgataatt |
| 841 | attgccgcaa tcccgatggc cagccgaggc catggtgcta tactcttgac cctcacaccc |
| 901 | gctgggagta ctgtgcaatt aaaacatgcg ctgacaatac tatgaatgac actgatgttc |
| 961 | ctttggaaac aactgaatgc atccaaggtc aaggagaagg ctacaggggc actgtcaata |
| 1021 | ccatttggaa tggaattcca tgtcagcgtt gggattctca gtatcctcac gagcatgaca |
| 1081 | tgactcctga aaatttcaag tgcaaggacc tacgagaaaa ttactgccga aatccagatg |
| 1141 | ggtctgaatc accctggtgt tttaccactg atccaaacat ccgagttggc tactgctccc |
| 1201 | aaattccaaa ctgtgatatg tcacatggac aagattgtta tcgtgggaat ggcaaaaatt |
| 1261 | atatgggcaa cttatcccaa acaagatctg gactaacatg ttcaatgtgg gacaagaaca |
| 1321 | tggaagactt acatcgtcat atcttctggg aaccagatgc aagtaagctg aatgagaatt |
| 1381 | actgccgaaa tccagatgat gatgctcatg gaccctggtg ctacacggga aatccactca |
| 1441 | ttccttggga ttattgccct atttctcgtt gtgaaggtga taccacacct acaatagtca |
| 1501 | atttagacca tcccgtaata tcttgtgcca aaacgaaaca attgcgagtt gtaaatggga |
| 1561 | ttccaacacg aacaaacata ggatggatgg ttagtttgag atacagaaat aaacatatct |
| 1621 | gcggaggatc attgataaag gagagttggg ttcttactgc acgacagtgt ttcccttctc |
| 1681 | gagacttgaa agattatgaa gcttggcttg gaattcatga tgtccacgga agaggagatg |
| 1741 | agaaatgcaa acaggttctc aatgtttccc agctggtata tggccctgaa ggatcagatc |
| 1801 | tggttttaat gaagcttgcc aggcctgctg tcctggatga ttttgttagt acgattgatt |
| 1861 | tacctaatta tggatgcaca attcctgaaa agaccagttg cagtgtttat ggctggggct |
| 1921 | acactggatt gatcaactat gatggcctat tacgagtggc acatctctat ataatgggaa |
| 1981 | atgagaaatg cagccagcat catcgaggga aggtgactct gaatgagtct gaaatatgtg |
| 2041 | ctggggctga aaagattgga tcaggaccat gtgaggggga ttatggtggc ccacttgttt |
| 2101 | gtgagcaaca taaaatgaga atggttcttg gtgtcattgt tcctggtcgt ggatgtgcca |
| 2161 | ttccaaatcg tcctggtatt tttgtccgag tagcatatta tgcaaaatgg atacacaaaa |
| 2221 | ttattttaac atataaggta ccacagtcat agctgaagta agtgtgtctg aagcacccac |
| 2281 | caatacaact gtcttttaca tgaagatttc agagaatgtg gaatttaaaa tgtcacttac |
| 2341 | aacaatccta agacaactac tggagagtca tgtttgttga aattctcatt aatgtttatg |
| 2401 | ggtgttttct gttgttttgt ttgtcagtgt tattttgtca atgttgaagt gaattaaggt |
| 2461 | acatgcaagt gtaataacat atctcctgaa gatacttgaa tggattaaaa aaacacacag |
| 2521 | gtatatttgc tggatgataa agatttcatg ggaaaaaaaa tcaattaatc tgtctaagct |
| 2581 | gctttctgat gttggtttct taataatgag taaaccacaa attaaatgtt attttaacct |
| 2641 | caccaaaaca atttatacct tgtgtcccta aattgtagcc ctatattaaa ttatattaca |
| 2701 | tttc |

The amino acid sequence of human HGF is provided by GenBank Accession No. AAA64239.1 and is shown below (SEQ ID NO: 18). The signal peptide is shown in underlined font.

| (SEQ ID NO: 18) | |
|---|---|
| 1 | mwvtkllpal llqhvllhll llpiaipyae gqrkrrntih efkksakttl ikidpalkik |
| 61 | tkkvntadqc anrctrnkgl pftckafvfd karkqclwfp fasmssgvkk efghefdlye |
| 121 | nkdyirncii gkgrsykgtv sitksgikcq pwssmipheh sflpssyrgk dlqeaycrap |
| 181 | rgeeggpwcf tsnpevryev cdipqcseve cmtcagesyr glmdhtesgk icqrwdhqtp |
| 241 | hrhkflpery pdkgfddnyc rnpdgqprpw cytldphtrw eycaiktcad ntmndtdvpl |
| 301 | etteciqgqg egyrgtvnti wngipcqrwd sqyphehdmt penfkckdlr eaycrapdgs |
| 361 | espwcfttdp nirvgycsqi pncdmshgqd cyrgagkaym gnlsqtrsgl tcsmwdknme |
| 421 | dlhrhifwep dasklnenyc rnpdddahgp wcytgaplip wdycpisrce gdttptivnl |
| 481 | dhpviscakt kqlrvvngip trtnigwmvs lryrakhicg gslikeswvl tarqcfpsrd |
| 541 | lkdyeawlgi hdvhgrgdek ckqvlnvsql vygpegsdlv lmklarpavl ddfvstidlp |
| 601 | nygctipekt scsvygwgyt glinydgllr vahlyimgae kcsqhhrgkv tlneseicag |
| 661 | aekigsgpce gdyggplvce qhkmrmvlgv ivpgrgcaip arpgifvrva yyakwihkii |
| 721 | ltykvpqs |

The mRNA sequence of human WNTSA is provided by GenBank Accession No. NM_003392.4 and is shown below (SEQ ID NO: 19). The start and stop codons are bolded and underlined.

| (SEQ ID NO: 19) | |
|---|---|
| 1 | actaactcgc ggctgcagga tcagcgtctg gaagcagacg tttcggctac agacccagag |
| 61 | aggaggagct ggagatcagg aggcgtgagc cgccaagagt ttgcagaatc tgtggtgtga |
| 121 | atgaactggg ggcacctggg cgcacagatc gccccccttc ccccgccccg ggccacagtt |
| 181 | gagtagtggt acattttttt caccctcttg tgaagaattt ctttttatta ttatttgtcg |
| 241 | taaggtcttt tgcacaatca cgcccacatt tggggttgga aagccctaat taccgccgtc |
| 301 | gctgatggac gttggaaacg gagcgcctct ccgtggaaca gttgcctgcg cgccctcgcc |
| 361 | ggaccggcgg ctccctagtt gcgccccgac caggccctgc ccttgctgcc ggctcgcgcg |
| 421 | cgtccgcgcc ccctccattc ctgggcgcat cccagctctg ccccaactcg ggagtccagg |
| 481 | cccgggcgcc agtgcccgct tcagctccgg ttcactgcgc ccgccggacg cgcgccggag |
| 541 | gactccgcag ccctgctcct gaccgtcccc ccaggcttaa cccggtcgct ccgctcggat |
| 601 | tcctcggctg cgctcgctcg ggtggcgact tcctccccgc gccccctccc cctcgccatg |
| 661 | aagaagtcca ttggaatatt aagcccagga gttgctttgg ggatggctgg aagtgcaatg |
| 721 | tcttccaagt tcttcctagt ggctttggcc atatttttct ccttcgccca ggttgtaatt |
| 781 | gaagccaatt cttggtggtc gctaggtatg aataaccctg ttcagatgtc agaagtatat |
| 841 | attataggag cacagcctct ctgcagccaa ctggcaggac tttctcaagg acagaagaaa |
| 901 | ctgtgccact tgtatcagga ccacatgcag tacatcggag aaggcgcgaa gacaggcatc |
| 961 | aaagaatgcc agtatcaatt ccgacatcga aggtggaact gcagcactgt ggataacacc |
| 1021 | tctgtttttg gcagggtgat gcagataggc agccgcgaga cggccttcac atacgcggtg |
| 1081 | agcgcagcag gggtggtgaa cgccatgagc cgggcgtgcc gcgagggcga gctgtccacc |
| 1141 | tgcggctgca gccgcgccgc gcgccccaag gacctgccgc gggactggct ctggggcggc |
| 1201 | tgcggcgaca acatcgacta tggctaccgc tttgccaagg agttcgtgga cgcccgcgag |
| 1261 | cgggagcgca tccacgccaa gggctcctac gagagtgctc gcatcctcat gaacctgcac |
| 1321 | aacaacgagg ccggccgcag gacggtgtac aacctggctg atgtggcctg caagtgccat |
| 1381 | ggggtgtccg gctcatgtag cctgaagaca tgctggctgc agctggcaga cttccgcaag |
| 1441 | gtgggtgatg ccctgaagga gaagtacgac agcgcggcgg ccatgcggct caacagccgg |
| 1501 | ggcaagttgg tacaggtcaa cagccgcttc aactcgccca ccacacaaga cctggtctac |
| 1561 | atcgacccca gccctgacta ctgcgtgcgc aatgagagca ccggctcgct gggcacgcag |
| 1621 | ggccgcctgt gcaacaagac gtcggagggc atggatggct gcgagctcat gtgctgcggc |
| 1681 | cgtggctacg accagttcaa gaccgtgcag acggagcgct gccactgcaa gttccactgg |
| 1741 | tgctgctacg tcaagtgcaa gaagtgcacg gagatcgtgg accagtttgt gtgcaagtag |
| 1801 | tgggtgccac ccagcactca gccccgctcc caggacccgc ttatttatag aaagtacagt |
| 1861 | gattctggtt tttggttttt agaaatattt tttatttttc cccaagaatt gcaaccggaa |
| 1921 | ccattttttt tcctgttacc atctaagaac tctgtggttt attattaata ttataattat |
| 1981 | tatttggcaa taatgggggt gggaaccaag aaaaatattt attttgtgga tctttgaaaa |
| 2041 | ggtaatacaa gacttctttt gatagtatag aatgaagggg aaataacaca taccctaact |
| 2101 | tagctgtgtg gacatggtac acatccagaa ggtaaagaaa tacattttct ttttctcaaa |
| 2161 | tatgccatca tatgggatgg gtaggttcca gttgaaagag ggtggtagaa atctattcac |
| 2221 | aattcagctt ctatgaccaa aatgagttgt aaattctctg gtgcaagata aaaggtcttg |
| 2281 | ggaaaacaaa acaaaacaaa acaaacctcc cttccccagc agggctgcta gcttgctttc |
| 2341 | tgcattttca aaatgataat ttacaatgga aggacaagaa tgtcatattc tcaaggaaaa |
| 2401 | aaggtatatc acatgtctca ttctcctcaa atattccatt tgcagacaga ccgtcatatt |
| 2461 | ctaatagctc atgaaatttg ggcagcaggg aggaaagtcc ccagaaatta aaaaatttaa |
| 2521 | aactcttatg tcaagatgtt gatttgaagc tgttataaga attaggattc cagattgtaa |
| 2581 | aaagatcccc aaatgattct ggacactaga tttttttgtt tggggaggtt ggcttgaaca |
| 2641 | taaatgaaaa tatcctgtta ttttcttagg gatacttggt tagtaaatta taatagtaaa |
| 2701 | aataatacat gaatcccatt cacaggttct cagcccaagc aacaaggtaa ttgcgtgcca |
| 2761 | ttcagcactg caccagagca gacaacctat ttgaggaaaa acagtgaaat ccaccttcct |
| 2821 | cttcacactg agccctctct gattcctccg tgttgtgatg tgatgctggc cacgtttcca |
| 2881 | aacggcagct ccactgggtc ccctttggtt gtaggacagg aaatgaaaca ttaggagctc |
| 2941 | tgcttggaaa acagttcact acttagggat ttttgtttcc taaaactttt attttgagga |
| 3001 | gcagtagttt tctatgtttt aatgacagaa cttggctaat ggaattcaca gaggtgttgc |
| 3061 | agcgtatcac tgttatgatc ctgtgtttag attatccact catgcttctc ctattgtact |
| 3121 | gcaggtgtac cttaaaactg ttcccagtgt acttgaacag ttgcatttat aaggggggaa |
| 3181 | atgtggttta atggtgcctg atatctcaaa gtcttttgta cataacatat atatatatat |
| 3241 | acatatatat aaatataaat ataaatatat ctcattgcag ccagtgattt agatttacag |
| 3301 | tttactctgg ggttatttct ctgtctagag cattgttgtc cttcactgca gtccagttgg |
| 3361 | gattattcca aaagtttttt gagtcttgag cttgggctgt ggccctgctg tgatcatacc |
| 3421 | ttgagcacga cgaagcaacc ttgtttctga ggaagcttga gttctgactc actgaaatgc |
| 3481 | gtgttgggtt gaagatatct tttttctttt ctgcctcacc cctttgtctc caacctccat |
| 3541 | ttctgttcac tttgtggaga gggcattact tgttcgttat agacatggac gttaagagat |
| 3601 | attcaaaact cagaagcatc agcaatgttt ctcttttctt agttcattct gcagaatgga |
| 3661 | aacccatgcc tattagaaat gacagtactt attaattgag tccctaagga atattcagcc |
| 3721 | cactacatag atagcttttt tttttttttt tttaataagg acacctcttt ccaaacagtg |
| 3781 | ccatcaaata tgttcttatc tcagacttac gttgttttaa aagtttggaa agatacacat |
| 3841 | ctttcatacc ccccttaggc aggttggctt tcatatcacc tcagccaact gtggctctta |
| 3901 | atttattgca taatgatatt cacatcccct cagttgcagt gaattgtgag caaaagatct |
| 3961 | tgaaagcaaa aagcactaat tagtttaaaa tgtcactttt ttggttttta ttatacaaaa |
| 4021 | accatgaagt acttttttta tttgctaaat cagattgttc ctttttagtg actcatgttt |
| 4081 | atgaagagag ttgagtttaa caatcctagc ttttaaaaga aactatttaa tgtaaaatat |
| 4141 | tctacatgtc attcagatat tatgtatatc ttctagcctt tattctgtac ttttaatgta |
| 4201 | catatttctg tcttgcgtga tttgtatatt tcactggttt aaaaaacaaa catcgaaagg |
| 4261 | cttatgccaa atggaagata gaatataaaa taaaacgtta cttgtatatt ggtaagtggt |
| 4321 | ttcaattgtc cttcagataa ttcatgtgga gatttttgga gaaaccatga cggatagttt |
| 4381 | aggatgacta catgtcaaag taataaaaga gtggtgaatt ttaccaaaac caagctattt |
| 4441 | ggaagcttca aaaggtttct atatgtaatg gaacaaaagg ggaattctct tttcctatat |
| 4501 | atgttcctta caaaaaaaaa aaaaaaagaa atcaagcaga tggcttaaag ctggttatag |
| 4561 | gattgctcac attcttttag cattatgcat gtaacttaat tgttttagag cgtgttgctg |
| 4621 | ttgtaacatc ccagagaaga atgaaaaggc acatgctttt atccgtgacc agatttttag |
| 4681 | tccaaaaaaa tgtatttttt tgtgtgttta ccactgcaac tattgcacct ctctatttga |
| 4741 | atttactgtg gaccatgtgt ggtgtctcta tgccctttga aagcagtttt tataaaaaga |
| 4801 | aagcccgggt ctgcagagaa tgaaaactgg ttggaaacta aaggttcatt gtgttaagtg |
| 4861 | caattaatac aagttattgt gcttttcaaa aatgtacacg gaaatctgga cagtgctcca |
| 4921 | cagattgata cattagcctt tgctttttct ctttccggat aaccttgtaa catattgaaa |
| 4981 | ccttttaagg atgccaagaa tgcattattc cacaaaaaaa cagcagacca acatatagag |
| 5041 | tgtttaaaat agcatttctg ggcaaattca aactcttgtg gttctaggac tcacatctgt |
| 5101 | ttcagttttt cctcagttgt atattgacca gtgttcttta ttgcaaaaac atatacccga |
| 5161 | tttagcagtg tcagcgtatt ttttcttctc atcctggagc gtattcaaga tcttcccaat |
| 5221 | acaagaaaat taataaaaaa tttatatata ggcagcagca aaagagccat gttcaaaata |
| 5281 | gtcattatgg gctcaaatag aaagaagact tttaagtttt aatccagttt atctgttgag |
| 5341 | ttctgtgagc tactgacctc ctgagactgg cactgtgtaa gttttagttg cctaccctag |
| 5401 | ctcttttctc gtacaatttt gccaatacca agtttcaatt tgtttttaca aaacattatt |
| 5461 | caagccacta gaattatcaa atatgacgct atagcagagt aaatactctg aataagagac |
| 5521 | cggtactagc taactccaag agatcgttag cagcatcagt ccacaaacac ttagtggccc |
| 5581 | acaatatata gagagataga aaaggtagtt ataacttgaa gcatgtattt aatgcaaata |
| 5641 | ggcacgaagg cacaggtcta aaatactaca ttgtcactgt aagctatact tttaaaatat |
| 5701 | ttattttttt taaagtattt tctagtcttt tctctctctg tggaatggtg aaagagagat |
| 5761 | gccgtgtttt gaaagtaaga tgatgaaatg aatttttaat tcaagaaaca ttcagaaaca |
| 5821 | taggaattaa aacttagaga aatgatctaa tttccctgtt cacacaaact ttacacttta |
| 5881 | atctgatgat tggatatttt attttagtga aacatcatct tgttagctaa ctttaaaaaa |
| 5941 | tggatgtaga atgattaaag gttggtatga ttttttttta atgtatcagt ttgaacctag |
| 6001 | aatattgaat taaaatgctg tctcagtatt ttaaaagcaa aaaaggaatg gaggaaaatt |
| 6061 | gcatcttaga ccatttttat atgcagtgta caatttgctg ggctagaaat gagataaaga |
| 6121 | ttatttattt ttgttcatat cttgtacttt tctattaaaa tcattttatg aaatccaaaa |
| 6181 | aaaaaaaaaa aaaa |

The amino acid sequence of human WNT5A is provided by GenBank Accession No. NP_003383.2 and is shown below (SEQ ID NO: 20).

| (SEQ ID NO: 20) | |
|---|---|
| 1 | mkksigilsp gvalgmagsa msskfflval aiffsfaqvv ieanswwslg mnnpvqmsev |
| 61 | yiigaqplcs qlaglsqgqk klchlyqdhm qyigegaktg ikecqyqfrh rrwncstvdn |
| 121 | tsvfgrvmqi gsretaftya vsaagvvnam sracregels tcgcsraarp kdlprdwlwg |
| 181 | gcgdnidygy rfakefvdar ererihakgs yesarilmnl hnneagrrtv ynladvackc |
| 241 | hgvsgscslk tcwlqladfr kvgdalkeky dsaaamrlns rgklvqvnsr fnspttqdlv |
| 301 | yidpspdycv rnestgslgt qgrlcnktse gmdgcelmcc grgydqfktv qterchckfh |
| 361 | wccyvkckkc teivdqfvck |

The mRNA sequence of human CCL2 is provided by GenBank Accession No. NM_002982.3 and is shown below (SEQ ID NO: 21). The start and stop codons are bolded and underlined.

| (SEQ ID NO: 21) | |
|---|---|
| 1 | gaggaaccga gaggctgaga ctaacccaga aacatccaat tctcaaactg aagctcgcac |
| 61 | tctcgcctcc aqcatgaaaq tctctgccgc ccttctgtgc ctgctgctca tagcagccac |
| 121 | cttcattccc caagggctcg ctcagccaga tgcaatcaat gccccagtca cctgctgtta |
| 181 | taacttcacc aataggaaga tctcagtgca gaggctcgcg agctatagaa gaatcaccag |
| 241 | cagcaagtgt cccaaagaag ctgtgatctt caagaccatt gtggccaagg agatctgtgc |
| 301 | tgaccccaag cagaagtggg ttcaggattc catggaccac ctggacaagc aaacccaaac |
| 361 | tccgaagact tgaacactca ctccacaacc caagaatctg cagctaactt attttcccct |
| 421 | agctttcccc agacaccctg ttttatttta ttataatgaa ttttgtttgt tgatgtgaaa |
| 481 | cattatgcct taagtaatgt taattcttat ttaagttatt gatgttttaa gtttatcttt |
| 541 | catggtacta gtgtttttta gatacagaga cttggggaaa ttgcttttcc tcttgaacca |
| 601 | cagttctacc cctgggatgt tttgagggtc tttgcaagaa tcattaatac aaagaatttt |
| 661 | ttttaacatt ccaatgcatt gctaaaatat tattgtggaa atgaatattt tgtaactatt |
| 721 | acaccaaata aatatatttt tgtacaaaaa aaaaaaaaaa |

The amino acid sequence of human CCL2 is provided by GenBank Accession No. NP_002973.1 and is shown below (SEQ ID NO: 22). The predicted signal peptide is underlined.

| (SEQ ID NO: 22) | |
|---|---|
| 1 | mkvsaallcl lliaatfipq glaqpdaina pvtccynftn rkisvqrlas yrritsskcp |
| 61 | keavifktiv akeicadpkq kwvqdsmdhl dkqtqtpkt |

The mRNA sequence of human colony stimulating factor 2 (CSF2) is provided by GenBank Accession No. NM_000758.3 and is shown below (SEQ ID NO: 23). The start and stop codons are bolded and underlined.

| (SEQ ID NO: 23) | |
|---|---|
| 1 | acacagagag aaaggctaaa gttctctgga ggatgtggct gcagagcctg ctgctcttgg |
| 611 | gcactgtggc ctgcagcatc tctgcacccg cccgctcgcc cagccccagc acgcagccct |
| 121 | gggagcatgt gaatgccatc caggaggccc ggcgtctcct gaacctgagt agagacactg |
| 181 | ctgctgagat gaatgaaaca gtagaagtca tctcagaaat gtttgacctc caggagccga |
| 241 | cctgcctaca gacccgcctg gagctgtaca agcagggcct gcggggcagc ctcaccaagc |
| 301 | tcaagggccc cttgaccatg atggccagcc actacaagca gcactgccct ccaaccccgg |
| 361 | aaacttcctg tgcaacccag attatcacct ttgaaagttt caaagagaac ctgaaggact |
| 421 | ttctgcttgt catccccttt gactgctggg agccagtcca ggagtgagac cggccagatg |
| 481 | aggctggcca agccggggag ctgctctctc atgaaacaag agctagaaac tcaggatggt |
| 541 | catcttggag ggaccaaggg gtgggccaca gccatggtgg gagtggcctg gacctgccct |
| 601 | gggccacact gaccctgata caggcatggc agaagaatgg gaatatttta tactgacaga |
| 661 | aatcagtaat atttatatat ttatattttt aaaatattta tttatttatt tatttaagtt |
| 721 | catattccat atttattcaa gatgttttac cgtaataatt attattaaaa atatgcttct |
| 781 | acttgaaaaa aaaaaaaaaa |

The amino acid sequence of human colony stimulating factor 2 (CSF2) is provided by GenBank Accession No. NP_000749.2 and is shown below (SEQ ID NO: 24). The signal peptide is underlined.

| (SEQ ID NO: 24) | |
|---|---|
| 1 | mwlqsllllg tvacsisapa rspspstqpw ehvnaiqear rllnlsrdta aemnetvevi |
| 61 | semfdlqept clqtrlelyk qglrgsltkl kgpltmmash ykqhcpptpe tscatqiitf |
| 121 | esfkenlkdf llvipfdcwe pvqe |

The mRNA sequence of human connective tissue growth factor (CTGF) is provided by GenBank Accession No. NM_001901.2 and is shown below (SEQ ID NO: 25). The start and stop codons are bolded and underlined.

| (SEQ ID NO: 25) | |
|---|---|
| 1 | aaactcacac aacaactctt ccccgctgag aggagacagc cagtgcgact ccaccctcca |
| 61 | gctcgacggc agccgccccg gccgacagcc ccgagacgac agcccggcgc gtcccggtcc |
| 121 | ccacctccga ccaccgccag cgctccaggc cccgccgctc cccgctcgcc gccaccgcgc |
| 181 | cctccgctcc gcccgcagtg ccaaccatga ccgccgccag tatgggcccc gtccgcgtcg |
| 241 | ccttcgtggt cctcctcgcc ctctgcagcc ggccggccgt cggccagaac tgcagcgggc |
| 301 | cgtgccggtg cccggacgag ccggcgccgc gctgcccggc gggcgtgagc ctcgtgctgg |
| 361 | acggctgcgg ctgctgccgc gtctgcgcca agcagctggg cgagctgtgc accgagcgcg |
| 421 | acccctgcga cccgcacaag ggcctcttct gtgacttcgg ctccccggcc aaccgcaaga |
| 481 | tcggcgtgtg caccgccaaa gatggtgctc cctgcatctt cggtggtacg gtgtaccgca |
| 541 | gcggagagtc cttccagagc agctgcaagt accagtgcac gtgcctggac ggggcggtgg |
| 601 | gctgcatgcc cctgtgcagc atggacgttc gtctgcccag ccctgactgc cccttcccga |
| 661 | ggagggtcaa gctgcccggg aaatgctgcg aggagtgggt gtgtgacgag cccaaggacc |
| 721 | aaaccgtggt tgggcctgcc ctcgcggctt accgactgga agacacgttt ggcccagacc |
| 781 | caactatgat tagagccaac tgcctggtcc agaccacaga gtggagcgcc tgttccaaga |
| 841 | cctgtgggat gggcatctcc acccgggtta ccaatgacaa cgcctcctgc aggctagaga |
| 901 | agcagagccg cctgtgcatg gtcaggcctt gcgaagctga cctggaagag aacattaaga |
| 961 | agggcaaaaa gtgcatccgt actcccaaaa tctccaagcc tatcaagttt gagctttctg |
| 1021 | gctgcaccag catgaagaca taccgagcta aattctgtgg agtatgtacc gacggccgat |
| 1081 | gctgcacccc ccacagaacc accaccctgc cggtggagtt caagtgccct gacggcgagg |
| 1141 | tcatgaagaa gaacatgatg ttcatcaaga cctgtgcctg ccattacaac tgtcccggag |
| 1201 | acaatgacat ctttgaatcg ctgtactaca ggaagatgta cggagacatg gcatgaagcc |
| 1261 | agagagtgag agacattaac tcattagact ggaacttgaa ctgattcaca tctcattttt |
| 1321 | ccgtaaaaat gatttcagta gcacaagtta tttaaatctg tttttctaac tgggggaaaa |
| 1381 | gattcccacc caattcaaaa cattgtgcca tgtcaaacaa atagtctatc aaccccagac |
| 1441 | actggtttga agaatgttaa gacttgacag tggaactaca ttagtacaca gcaccagaat |
| 1501 | gtatattaag gtgtggcttt aggagcagtg ggagggtacc agcagaaagg ttagtatcat |
| 1561 | cagatagcat cttatacgag taatatgcct gctatttgaa gtgtaattga gaaggaaaat |
| 1621 | tttagcgtgc tcactgacct gcctgtagcc ccagtgacag ctaggatgtg cattctccag |
| 1681 | ccatcaagag actgagtcaa gttgttcctt aagtcagaac agcagactca gctctgacat |
| 1741 | tctgattcga atgacactgt tcaggaatcg gaatcctgtc gattagactg gacagcttgt |
| 1801 | ggcaagtgaa tttgcctgta acaagccaga ttttttaaaa tttatattgt aaatattgtg |
| 1861 | tgtgtgtgtg tgtgtgtata tatatatata tgtacagtta tctaagttaa tttaaagttg |
| 1921 | tttgtgcctt tttatttttg tttttaatgc tttgatattt caatgttagc ctcaatttct |
| 1981 | gaacaccata ggtagaatgt aaagcttgtc tgatcgttca aagcatgaaa tggatactta |
| 2041 | tatggaaatt ctgctcagat agaatgacag tccgtcaaaa cagattgttt gcaaagggga |
| 2101 | ggcatcagtg tccttggcag gctgatttct aggtaggaaa tgtggtagcc tcacttttaa |
| 2161 | tgaacaaatg gcctttatta aaaactgagt gactctatat agctgatcag ttttttcacc |
| 2221 | tggaagcatt tgtttctact ttgatatgac tgtttttcgg acagtttatt tgttgagagt |
| 2281 | gtgaccaaaa gttacatgtt tgcacctttc tagttgaaaa taaagtgtat attttttcta |
| 2341 | taaaaaaaaa aaaaaaaa |

The amino acid sequence of human connective tissue growth factor (CTGF) is provided by GenBank Accession No. NP_001892.1 and is shown below (SEQ ID NO: 26). The predicted signal peptide is underlined.

| (SEQ ID NO: 26) | |
|---|---|
| 1 | mtaasmgpvr vafvvllalc srpavgqncs gpcrcpdepa prcpagvslv ldgcgccrvc |
| 61 | akqlgelcte rdpcdphkgl fcdfgspanr kigvctakdg apcifggtvy rsgesfqssc |
| 121 | kyqctcldga vgcmplcsmd vrlpspdcpf prrvklpgkc ceewvcdepk dqtvvgpala |
| 181 | ayrledtfgp dptmirancl vqttewsacs ktcgmgistr vtndnascrl ekqsrlcmvr |
| 241 | pceadleeni kkgkkcirtp kiskpikfel sgctsmktyr akfcgvctdg rcctphrttt |
| 301 | lpvefkcpdg evmkknmmfi ktcachyncp gdndifesly yrkmygdma |

The mRNA sequence of human transgelin (TAGLN) is provided by GenBank Accession No. NM_001001522.1 and is shown below (SEQ ID NO: 27). The start and stop codons are bolded and underlined.

| (SEQ ID NO: 27) | |
|---|---|
| 1 | tcaccacggc ggcagccctt taaacccctc acccagccag cgccccatcc tgtctgtccg |
| 61 | aacccagaca caagtcttca ctccttcctg cgagccctga ggaagccttg tgagtgcatt |
| 121 | ggctggggct tggagggaag ttgggctgga gctggacagg agcagtgggt gcatttcagg |
| 181 | caggctctcc tgaggtccca ggcgccagct ccagctccct ggctagggaa acccaccctc |
| 241 | tcagtcagca tgggggccca agctccaggc agggtgggct ggatcactag cgtcctggat |
| 301 | ctctctcaga ctgggcagcc ccgggctcat tgaaatgccc cggatgactt ggctagtgca |
| 361 | gaggaattga tggaaaccac cggggtgaga gggaggctcc ccatctcagc cagccacatc |
| 421 | cacaaggtgt gtgtaagggt gcaggcgccg gccggttagg ccaaggctct actgtctgtt |
| 481 | gcccctccag gagaacttcc aaggagcttt ccccagacat ggccaacaag ggtccttcct |
| 541 | atggcatgag ccgcgaagtg cagtccaaaa tcgagaagaa gtatgacgag gagctggagg |
| 601 | agcggctggt ggagtggatc atagtgcagt gtggccctga tgtgggccgc ccagaccgtg |
| 661 | ggcgcttggg cttccaggtc tggctgaaga atggcgtgat tctgagcaag ctggtgaaca |
| 721 | gcctgtaccc tgatggctcc aagccggtga aggtgcccga gaacccaccc tccatggtct |
| 781 | tcaagcagat ggagcaggtg gctcagttcc tgaaggcggc tgaggactat ggggtcatca |
| 841 | agactgacat gttccagact gttgacctct ttgaaggcaa agacatggca gcagtgcaga |
| 901 | ggaccctgat ggctttgggc agcttggcag tgaccaagaa tgatgggcac taccgtggag |
| 961 | atcccaactg gtttatgaag aaagcgcagg agcataagag ggaattcaca gagagccagc |
| 1021 | tgcaggaggg aaagcatgtc attggccttc agatgggcag caacagaggg gcctcccagg |
| 1081 | ccggcatgac aggctacgga cgacctcggc agatcatcag ttagagcgga gagggctagc |
| 1141 | cctgagcccg gccctccccc agctccttgg ctgcagccat cccgcttagc ctgcctcacc |
| 1201 | cacacccgtg tggtaccttc agccctggcc aagctttgag gctctgtcac tgagcaatgg |
| 1261 | taactgcacc tgggcagctc ctccctgtgc ccccagcctc agcccaactt cttacccgaa |
| 1321 | agcatcactg ccttggcccc tccctcccgg ctgcccccat cacctctact gtctcctccc |
| 1381 | tgggctaagc aggggagaag cgggctgggg gtagcctgga tgtgggccaa gtccactgtc |
| 1441 | ctccttggcg gcaaaagccc attgaagaag aaccagccca gcctgccccc tatcttgtcc |
| 1501 | tggaatattt ttggggttgg aactcaaaaa aaaaaaaaaa aaatcaatct tttctcaaaa |
| 1561 | aaaaaaaaaa aaaa |

The amino acid sequence of human transgelin (TAGLN) is provided by GenBank Accession No. NP_001001522.1 and is shown below (SEQ ID NO: 28).

| (SEQ ID NO: 28) | |
|---|---|
| 1 | mankgpsygm srevqskiek kydeeleerl vewiivqcgp dvgrpdrgrl gfqvwlkngv |
| 61 | ilsklvnsly pdgskpvkvp enppsmvfkq meqvaqflka aedygviktd mfqtvdlfeg |
| 121 | kdmaavqrtl malgslavtk ndghyrgdpn wfmkkaqehk reftesqlqe gkhviglqmg |
| 181 | rgasqagm tgygrprqii s |

In some examples, VEGF includes VEGFA, VEGFB, VEGFC, and/or VEGFD. Exemplary GenBank Accession Nos. of VEGFA include (amino acid) AAA35789.1 (GI:181971) and (nucleic acid) NM_001171630.1 (GI:284172472), incorporated herein by reference. Exemplary GenBank Accession Nos. of VEGFB include (nucleic acid) NM_003377.4 and (amino acid) NP_003368.1, incorporated herein by reference. Exemplary GenBank Accession Nos. of VEGFC include (nucleic acid) NM_005429.3 and (amino acid) NP_005420.1, incorporated herein by reference. Exemplary GenBank Accession Nos. of VEGFD include (nucleic acid) NM_004469.4 and (amino acid) NP_004460.1, incorporated herein by reference.

Exemplary GenBank Accession Nos. of FGF include (nucleic acid) U76381.2 and (amino acid) AAB18786.3, incorporated herein by reference.

The hydrogels and methods described herein promote skin repair and regeneration without the need for exogenous cytokines, growth factors or bioactive drugs, but instead by simply adjusting the stiffness of a material, e.g., wound dressing material, placed in/on/around a wound site. For example, different wound dressing materials with different mechanical properties are implanted according to the wound repair stage one intends to promote or diminish.

The process of wound healing comprises several phases: hemostasis, inflammation, proliferation, and remodeling. Upon injury (e.g., to the skin), platelets aggregate at the site of injury to from a clot in order to reduce bleeding. This process is called hemostasis. In the inflammation phase, white blood cells remove bacteria and cell debris from the wound. In the proliferation phase, angiogenesis (formation of new blood vessels by vascular endothelial cells) occurs, as does collagen deposition, tissue formation, epithelialization, and wound contraction at the site of the wound. To form tissue at the site of the wound, fibroblasts grow to form a new extracellular matrix by secreting proteins such as fibronectin and collagen. Re-epithelialization also occurs in which epithelial cells proliferate and cover the site of the wound in order to cover the newly formed tissue. In order to cause wound contraction, myofibroblasts decrease the size of the wound by contracting and bringing in the edges of the wound. In the remodeling phase, apoptosis occurs to remove unnecessary cells at the site of the wound. One or more of these phases in the process of wound healing is disrupted or delayed in non-healing/slow-healing wounds, e.g., due to diabetes, old age, or infections.

Following a skin lesion, disruption of the tissue architecture leads to a dramatically altered mechanical context at the site of the wound (Wong et al. J Invest Dermatol. 2011; 131:2186-96). Mechanical cues in the wound microenvironment can guide the behavior of a milieu of infiltrating cells such as recruited immune cells (Wong et al. FASEB Journal. 2011; 25:4498-510.; McWhorter et al. Proceedings of the National Academy of Sciences. 2013; 110:17253-8) and fibroblasts (Wipff et al. J Cell Biol 2007; 179:1311-23). More broadly, mechanical cues are known to sponsor or hinder different stages of the wound repair response, from epithelial morphogenesis (Zhang et al. Nature. 2011; 471:99-103) to blood vessel formation (Boerckel et al. Proceedings of the National Academy of Sciences 2011; 108:674-80). Before the invention, importance of mechanical forces in the context of wound dressing design was often overlooked.

In some cases, the physicochemical properties of the hydrogel are manipulated to target healing at different stages of wound healing (Boateng et al. Journal of Pharmaceutical Sciences. 2008; 97:2892-923). For example, in some cases, it is beneficial to minimize the inflammatory stage of the healing response. A tissue lesion can cause acute inflammation, and resolution of this inflammatory phase must occur in order to achieve a complete and successful repair response. Systemic diseases such as diabetes, venous insufficiency, and/or infection, cause chronic inflammation, which is a hallmark of non-healing wounds and which impairs the healing process. See, e.g., Eming et al. J Invest Dermatol. 2007; 127:514-25. Depending on the type of wound and the subject (e.g., age, diseased/healthy), wound healing may progress differently and each stage of the wound healing process may take different amounts of time. As an example, early gestation fetus heals dermal wounds rapidly and scarlessly and in the absense of pro-inflammatory signals. See, e.g., Bullard K M, Longaker M T, Lorenz H P. Fetal Wound Healing: Current Biology. World J Surg. 2003; 27:54-61.

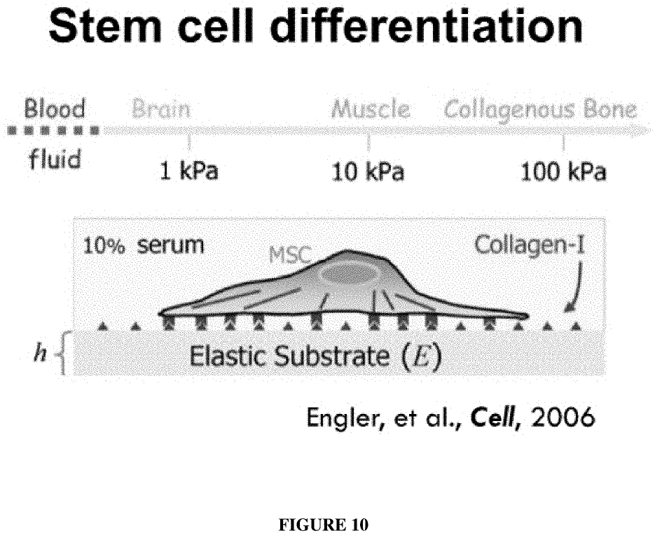

In some cases, the stiffness of the wound dressing materials matches the stiffness of structurally intact/healthy tissue (e.g., at the site of the wound prior to injury), which can vary depending on the type of injured tissue, site of injury, natural person-to-person variations, and/or age. For example, the stiffness can be tuned over the range of typical soft tissues (heart, lung, kidney, liver, muscle, neural, etc.) from elastic modulus ˜20 Pascals (fat) to ˜100,000 Pascals (skeletal muscle). Different tissue types are characterized by different stiffness, e.g., normal brain tissue has a shear modulus of approximately 200 Pascal. Cell growth/behavior also differs relative to the disease state of a given tissue, e.g., the shear modulus (a measure of stiffness) of normal mammary tissue is approximately 100 Pascal, whereas that of breast tumor tissue is approximately 2000 Pascal. Similarly, normal liver tissue has a shear modulus of approximately 300 Pascal compared to fibrotic liver tissue, which is characterized by a shear modulus of approximately 800 Pascal. Growth, signal transduction, gene or protein expression/secretion, as well as other physiologic parameters are altered in response to contact with different substrate stiffness and evaluated in response to contact with substrates characterized by mechanical properties that simulate different tissue types or disease states. A schematic illustrating the varying stiffnesses of substrates that lead to mesenchymal stem cell differentiation into various tissue types is shown in FIG. 10.

Skin is a multilayered, non-linear anisotropic material, which is under pre-stress in vivo. See, e.g., Annaidha et al. Journal of the Mechanical Behavior of Biomedical Materials. 2012; 5:139-48, incorporated herein by reference. Measuring the mechanical properties of skin is challenging, and the measured mechanical properties depend on the technique used. The Young's modulus (or storage modulus) of skin, E, has been reported to vary between 0.42 MPa and 0.85 MPa based on orsion tests, 4.6 MPa and 20 Mpa based on tensile tests, and between 0.05 MPa and 0.15 MPa based on suction tests. See, e.g., Pailler-Mattei Medical Engineering & Physics. 2008; 30:599-606, incorporated herein by reference. The skin's mechanical properties change as a person ages. Skin becomes thinner, stiffer, less tense, and less flexible with age. See, e.g., Fau et al. Int J Cosmet Sci. 2001; 23:353-62, incorporated herein by reference. For example, the Young's modulus (or storage modulus) of the skin doubles with age. See, e.g., Agache et al. Arch Dermatol Res. 1980; 269:221-32, incorporated herein by reference. Skin tension decreases with age, with tension being higher in a child (e.g., 21 N/mm²) and lower in the elderly adult (e.g., 17 N/mm²). The elasticity modulus also decreases with age, with the modulus being higher in children (e.g, 70 N/mm²) than in elderly adults (e.g., 60 N/mm²). Also, the mean ultimate skin deformation before bursting decreases from 75% for newborns to 60% for elderly adults. See, e.g., Pawlaczyk et al. Postep Dermatol Alergol 2013; 30:302-6, incorporated herein by reference.

Thus, the hydrogel materials, e.g., wound dressings, described herein are customized and specifically engineered to adopt the stiffness of a particular target age group. For example, the hydrogels comprise a stiffness that matches that of a tissue (e.g., cutaneous, mucous, bony, soft, vascular, or cartilaginous tissue) of a newborn, toddler, child, teenager, adult, middle-aged adult, or elderly adult. For example the stiffness of the hydrogels matches that of a tissue in a subject having an age of 0-2, 0-12, 2-6, 6-12, 13-18, 13-20, 0-18, 0-20, 20-50, 20-30, 20-40, 30-40, 30-50, 40-50, 50-110, 60-110, or 70-110 years. In some examples, hydrogels with a storage modulus of about 50-100 N/mm² are suitable for wound healing, e.g., of a cutaneous tissue, in a child, e.g., with an age of 18 years or less. In other examples, hydrogels with a storage modulus of about 40-80 N/mm² are suitable for wound healing, e.g., of a cutaneous tissue, in an adult, e.g., with an age of 18 years or older. Such hydrogels are made with the specified storage moduli by varying the components as described above.

The hydrogels/wound dressing materials of the invention modulate the expression of various proteins in cells (e.g., fibroblasts) at or surrounding the site of administration or the site of the injured tissue, e.g., a tissue that is undergoing the wound healing process. For example, the hydrogel modulates (e.g., upregulates or downregulates) the expression level of a protein involved in one or more of the phases of healing, e.g., hemostasis, inflammation, proliferation, and/or remodeling. For example, the modulated protein level enhances, accelerates, and/or diminishes a phase of healing.

For example, the hydrogel upregulates or downregulates the expression of an inflammation associated protein, e.g., IL-10 and/or COX-2, a cell adhesion or extracellular matrix protein, e.g., integrin a4 (ITGA4), metallopeptidase 1 (MMP1), or vitronectin (VTN), a collagen protein, e.g., Type IV (e.g., COL4A1 or COL4A3) or Type V (e.g., COL5A3) protein, or hepatocyte growth factor (HGF) or a member of the WNT gene family (WNTSA). For example, the expression is upregulated or downregulated at the polypeptide or mRNA level. The polypeptide or mRNA level of the protein is increased or decreased by at least 1.5-fold (e.g., at least 1.5, 2, 3, 4, 5, 6, 7, 8, 9, 10-fold, or greater) in tissues at or surrounding (e.g., within 5 cm, e.g., within 5, 4, 3, 2, 1, 0.5 cm or less of a border/perimeter of the hydrogel) the site of hydrogel administration compared to the level in the tissues prior to administration of the hydrogel.

In some embodiments, the IL-10 polypeptide or mRNA level is increased or decreased by at least 2-fold (e.g., at least 2, 3, 4, 5, 6, 7, 8, 9, 10-fold, or greater) in tissues at or surrounding (e.g., within 5 cm, e.g., within 5, 4, 3, 2, 1, 0.5 cm or less of a border/perimeter of the hydrogel) the site of hydrogel administration compared to the level in the tissues prior to administration of the hydrogel. In some cases, the COX-2 polypeptide or mRNA level is increased or decreased by at least 2-fold (e.g., at least 2, 3, 4, 5, 6, 7, 8, 9, 10, 12, 14, 18, 20-fold, or greater) in tissues at or surrounding (e.g., within 5 cm, e.g., within 5, 4, 3, 2, 1, 0.5 cm or less of a border/perimeter of the hydrogel) the site of hydrogel administration compared to the level in the tissues prior to administration of the hydrogel. In some examples, administration of the hydrogel reduces the level of proteins at a site of a wound that are involved in hemostasis, inflammation, proliferation, and/or remodeling, e.g., to prevent excessive clotting, inflammation, proliferative cells, and/or remodeling. For example, administration of the hydrogel reduces the level of inflammatory factors at a site of a wound, e.g., to minimize inflammation. In other examples, administration of the hydrogel enhances the level of proteins at a site of a wound that are involved in hemostasis, inflammation, proliferation, and/or remodeling.

In other embodiments, the hydrogel upregulates or downregulates the expression of an inflammation associated protein, e.g., CCL2, colony stimulating factor 2 (CSF2), connective tissue growth factor (CTGF), and/or transgelin (TAGLN) protein. The protein is upregulated or downregulated at the polypeptide or mRNA level, e.g., by at least 1.5-fold (e.g., at least 1.5, 2, 3, 4, 5, 6, 7, 8, 9, 10-fold, or greater) in tissues at or surrounding (e.g., within 5 cm, e.g., within 5, 4, 3, 2, 1, 0.5 cm or less of a border/perimeter of the hydrogel) the site of hydrogel administration compared to the level in the tissues prior to administration of the hydrogel.

The treatment of non-healing wounds, such as diabetic foot ulcers, requires a sophisticated therapy able to target ischemia, chronic infection, and adequate offloading (i.e., reduction of pressure) (Falanga et al. The Lancet. 2005; 366:1736-43). The biomaterial system, e.g., hydrogel, harnesses the mechanical properties of materials, e.g., advanced wound dressing materials, to treat non-healing wounds. In some examples, the hydrogels are used in concert with bioactive compositions, growth factor or cells (Kearney et al. Nature Materials. 2013; 12:1004-17).

Bioactive compositions are purified naturally-occurring, synthetically produced, or recombinant compounds, e.g., polypeptides, nucleic acids, small molecules, or other agents. The compositions described herein are purified. Purified compounds are at least 60% by weight (dry weight) the compound of interest. Preferably, the preparation is at least 75%, more preferably at least 90%, and most preferably at least 99%, by weight the compound of interest. Purity is measured by any appropriate standard method, for example, by column chromatography, polyacrylamide gel electrophoresis, or HPLC analysis.

This invention provides a method to control the behavior of fibroblasts involved in the wound healing response by tuning the storage modulus of a material, e.g., wound dressing material. Material systems have been developed to help understand how extracellular matrix mechanics regulates cell behaviors, from migration (Lo et al. Biophysical Journal. 2000; 79:144-52; Gardel et al. The Journal of cell biology. 2008; 183:999-1005) to differentiation (Engler et al. Cell. 2006; 126:677-89; Huebsch et al. Nature Materials. 2010; 9:518-26). However, these material systems do not allow the decoupling of matrix stiffness from altered ligand density, polymer density or scaffold architecture. Other types of materials, such as synthetic wound dressing materials are available, e.g., made exclusively of non-biological molecules or polymers. For example, a typical synthetic wound dressing is made of nonwoven fibers (e.g., composed of polyester, polyamide, polypropylene, polyurethane, and/or polytetrafluoethylene) and semipermeable filsm. An example of a synthetic skin substitute is BIOBRANE™, which has an inner layer of nylon mesh and an outer layer of silastic. See, e.g., Halim et al. Indian J Plast Surg. 2010; 43:S23-S8. Synthetic polymers allow for consistent variance and control of their composition and properties, but they lack naturally occurring matrix elements and natural tissue (e.g., skin) architecture that are required for cells to sense or respond to biological signals. Instead, the synthetic materials are a full artificial microenvironment/structure. This invention achieves this decoupling/separation by designing interpenetrating network (IPN) hydrogels, which are made up of two or more polymer networks that are not covalently bonded but at least partially interconnected (Wilkinson ADMaA. IUPAC. Compendium of Chemical Terminology. 2nd ed. Oxford, UK Blackwell Scientific Publications; 1997). For example, a biomaterial system composed of interpenetrating networks of collagen and alginate was developed. The alginate (e.g., sodium alginate) polymeric backbone presents no intrinsic cell-binding domains, but can be used to regulate gel mechanical properties. The collagen (e.g., collagen-I) presents specific peptide sequences recognized by cells surface receptors, and provides a substrate for cell adhesion that recreates the fibrous mesh of many in vivo contexts. Both of these components are biocompatible, biodegradable and widely used in the tissue engineering field. Encapsulated cells sense, adhere and pull on the collagen fibrils, and depending on the degree of crosslinking of the intercalated alginate mesh, cells will feel more or less resistance to deformation from the matrix. The alginate backbone is ionically crosslinked by ions, e.g., divalent cations (e.g., Ca⁺²). Thus, solely changing the concentration of Ca⁺² modulates the stiffness of the IPN. In some cases, dermal fibroblasts are recruited to the wound site for the synthesis, deposition, and remodeling of the new extracellular matrix (Singer et al. New England Journal of Medicine. 1999; 341:738-46). Dermal fibroblasts are an important cell player in the wound healing response.

The in vitro behavior of primary fibroblasts isolated from the dermis of healthy non-diabetic donors when encapsulated within IPNs of varying stiffness, partially mimicked the response of fibroblasts migrating into a wound site in vivo. In particular, primary fibroblasts isolated from the dermis of heathy adult patients were able to grow and survive within the interconnected network of the IPNs. Different storage moduli of different IPNs promoted dramatic changes in the morphology of fibroblasts, and triggered different wound healing genetic programs, including altered expression of inflammation mediators, e.g., IL10 and COX2. Enhancing the number of binding sites to which the fibroblasts could adhere did not subdue the effects of mechanics on cell spreading and contraction. Simply tuning the storage modulus of the hydrogels described herein, e.g., in cutaneous wound dressings, controls (e.g., promotes or hinders) the different stages of the wound healing response.

The term “isolated” used in reference to a cell type, e.g., a fibroblast, means that the cell is substantially free of other cell types or cellular material with which it naturally occurs. For example, a sample of cells of a particular tissue type or phenotype is “substantially pure” when it is at least 60% of the cell population. Preferably, the preparation is at least 75%, more preferably at least 90%, and most preferably at least 99% or 100%, of the cell population. Purity is measured by any appropriate standard method, for example, by fluorescence-activated cell sorting (FACS). Optionally, the hydrogel is seeded with two or more substantially pure populations of cells. The populations are spatially or physically separated, e.g., one population is encapsulated, or the cells are allowed to come into with one another. The hydrogel or structural support not only provides a surface upon which cells are seeded/attached but indirectly affects production/education of cell populations by housing a second (third, or several) cell population(s) with which a first population of cells associates (cell-cell adhesion).

In accordance with the methods of the invention, hydrogels described herein are administered, e.g., implanted, e.g., orally, systemically, sub- or trans-cutaneously, as an arterial stent, surgically, or via injection. In some examples, the hydrogels described herein are administered by routes such as injection (e.g., subcutaneous, intravenous, intracutaneous, percutaneous, or intramuscular) or implantation.

In one embodiment, administration of the device is mediated by injection or implantation into a wound or a site adjacent to the wound. For example, the wound is external or internal. In other embodiments, the hydrogel is placed over a wound, e.g, covering at least 50% (e.g., at least 50%, 60%, 70%, 80%, 90%, or 100%, or greater) of the surface area of the wound.

The hydrogels of the invention enhance the viability of passenger cells (e.g., fibroblasts, e.g., dermal fibroblasts, or epithelial cells such as mammary epithelial cells) and induce their outward migration to populate injured or defective bodily tissues to enhance the success of tissue regeneration and/or wound healing. Such a hydrogel that controls cell function and/or behavior, e.g., locomotion, growth, or survival, optionally also contains one or more bioactive compositions. The bioactive composition is incorporated into or coated onto the hydrogel. The hydrogel and/or bioactive composition temporally and spatially (directionally) controls egress of a resident cell (e.g., fibroblast) or progeny thereof. At the end of a treatment period, the hydrogel has released a substantial number of the passenger cells that were originally used to seed the hydrogel, e.g., there is a net efflux of passenger cells. For example, the hydrogel releases 10% or more (e.g., 10%, 20%, 30%, 40%, 50%, 60%, 70%, 80%, 90%, 100%, 200%, 300%, 400%, or more) of the seeded passenger cells by the end of a treatment period compared to at the commencement of treatment. In another example, the hydrogel contains 50% or less (e.g., 50%, 40%, 30%, 25%, 20%, 15%, 10%, 5%, 2.5%, 1%, or less) of the seeded passenger cells at the end of a treatment period compared to at the commencement of treatment. In some cases, a greater number of cells can be released than originally loaded if the cells proliferate after being placed in contact with the hydrogel.

In some cases, the hydrogels mediate modification and release of host cells from the material in vivo, thereby improving the function of cells that have resided in the hydrogels. For example, the hydrogel temporally and spatially (directionally) controls fibroblast migration. For example, the hydrogel mediates release of fibroblasts from the material in vivo.

Depending on the application for which the hydrogel is designed, the hydrogel regulates egress through its physical or chemical characteristics. For example, the hydrogel is differentially permeable, allowing cell egress only in certain physical areas of the hydrogel. The permeability of the hydrogel is regulated, for example, by selecting or engineering a material for greater or smaller pore size, density, polymer cross-linking, stiffness, toughness, ductility, or viscoelascticity. The hydrogel contains physical channels or paths through which cells can move more easily towards a targeted area of egress of the hydrogel or of a compartment within the hydrogel. The hydrogel is optionally organized into compartments or layers, each with a different permeability, so that the time required for a cell to move through the hydrogel is precisely and predictably controlled. Migration is also regulated by the degradation, de- or re-hydration, oxygenation, chemical or pH alteration, or ongoing self-assembly of the hydrogel. These processes are driven, e.g., by diffusion or cell-secretion of enzymes or other reactive chemicals.

Porosity of the hydrogel influences migration of the cells through the device and egress of the cells from the device. Pores are nanoporous, microporous, or macroporous. In some cases, the pores are a combination of these sizes. For example, the pores of the scaffold composition are large enough for a cell, e.g., fibroblast, to migrate through. For example, the diameter of nanopores are less than about 10 nm; micropores are in the range of about 100 nm-20 μm in diameter; and, macropores are greater than about 20 μm (preferably greater than about 100 μm and even more preferably greater than about 400 μm). In one example, the scaffold composition is macroporous with aligned pores of about 400-500 μm in diameter. In another example, the pores are nanoporous, e.g., about 20 μm to about 10 nm in diameter.

Alternatively or in addition, egress is regulated by a bioactive composition. By varying the concentration of growth factors, homing/migration factors, morphogens, differentiation factors, oligonucleotides, hormones, neurotransmitters, neurotransmitter or growth factor receptors, interferons, interleukins, chemokines, cytokines, colony stimulating factors, chemotactic factors, extracellular matrix components, adhesion molecules and other bioactive compounds in different areas of the hydrogel. The hydrogel controls and directs the migration of cells through its structure. Chemical affinities are used to channel cells towards a specific area of egress. For example, adhesion molecules are used to attract or retard the migration of cells. By varying the density and mixture of those bioactive substances, the hydrogel controls the timing of the migration and egress. In other cases, adhesion molecules are not attached to the alginate or collagen in the hydrogel. Rather, the collagen naturally contains cell adhesive properties and attracts/retards migration of cells. The density and mixture of the bioactive substances is controlled by initial doping levels or concentration gradient of the substance, by embedding the bioactive substances in hydrogel material with a known leaching rate, by release as the hydrogel material degrades, by diffusion from an area of concentration, by interaction of precursor chemicals diffusing into an area, or by production/excretion of compositions by resident support cells. The physical or chemical structure of the hydrogel also regulates the diffusion of bioactive agents through the hydrogel.

Signal transduction events that participate in the process of cell motility are initiated in response to cell growth and/or cell differentiation factors. Thus, the hydrogel optionally contains a second bioactive composition that is a growth factor, morphogen, differentiation factor, or chemoattractant. For example, the hydrogel includes vascular endothelial growth factor (VEGF), hepatocyte growth factor (HGF), or fibroblast growth factor 2 (FGF2) or a combination thereof. Other factors include hormones, neurotransmitters, neurotransmitter or growth factor receptors, interferons, interleukins, chemokines, MMP-sensitive substrate, cytokines, colony stimulating factors. Growth factors used to promote angiogenesis, bone regeneration, wound healing, and other aspects of tissue regeneration are listed herein and are used alone or in combination to induce colonization or regeneration of bodily tissues by cells that have migrated out of an implanted hydrogel.

The hydrogel is biocompatible. The hydrogel is bio-degradable/erodable or resistant to breakdown in the body. Preferably, the hydrogel degrades at a predetermined rate based on a physical parameter selected from the group consisting of temperature, pH, hydration status, and porosity, the cross-link density, type, and chemistry or the susceptibility of main chain linkages to degradation or it degrades at a predetermined rate based on a ratio of chemical polymers. For example, a calcium cross-linked gels composed of high molecular weight, high guluronic acid alginate degrade over several months (1, 2, 4, 6, 8, 10, 12 months) to years (1, 2, 5 years) in vivo, while a gel comprised of low molecular weight alginate, and/or alginate that has been partially oxidized, will degrade in a matter of weeks.

In one example, cells mediate degradation of the hydrogel matrix, i.e., the hydrogel is enzymatically digested by a composition elicited by a resident cell, and the egress of the cell is dependent upon the rate of enzymatic digestion of the hydrogel. In this case, polymer main chains or cross-links contain compositions, e.g., oligopeptides, that are substrates for collagenase or plasmin, or other enzymes produced by within or adjacent to the hydrogel.

The hydrogel are manufactured in their entirety in the absence of cells or can be assembled around or in contact with cells (the material is gelled or assembled around cells in vitro or in vivo in the presence of cells and tissues) and then contacted with cells to produce a cell-seeded structure. Alternatively, the hydrogel is manufactured in two or more (3, 4, 5, 6, . . . 10 or more) stages in which one layer or compartment is made and seeded with cells followed by the construction of a second, third, fourth or more layers, which are in turn seeded with cells in sequence. Each layer or compartment is identical to the others or distinguished from one another by the number, genotype, or phenotype of the seed cell population as well as distinct chemical, physical and biological properties. Prior to implantation, the hydrogel is contacted with purified populations cells or characterized mixtures of cells as described above. Preferably, the cells are human; however, the system is adaptable to other eukaryotic animal cells, e.g., canine, feline, equine, bovine, and porcine, as well as prokaryotic cells such as bacterial cells.

Therapeutic applications of the hydrogel include tissue generation, regeneration/repair, as well as augmentation of function of a mammalian bodily tissue in and around a wound.

In some cases, the cells (e.g., fibroblasts) remain resident in the hydrogel for a period of time, e.g., minutes; 0.2. 0.5, 1, 2, 4, 6, 12, 24 hours; 2, 4, 6, days; weeks (1-4), months (2, 4, 6, 8, 10, 12) or years, during which the cells are exposed to structural elements and, optionally, bioactive compositions that lead to proliferation of the cells, and/or a change in the activity or level of activity of the cells. The cells are contacted with or exposed to a deployment signal that induces egress of the optionally altered (re-educated or reprogrammed) cells and the cells migrate out of the hydrogel and into surrounding tissues or remote target locations.

The deployment signal is a composition such as protein, peptide, or nucleic acid. In some cases, the deployment signal is a nucleic acid molecule, e.g., a plasmid containing sequence encoding a protein that induces migration of the cell out of the hydrogel and into surrounding tissues. The deployment signal occurs when the cell encounters the plasmid in the hydrogel, the DNA becomes internalized in the cell (i.e., the cell is transfected), and the cell manufactures the gene product encoded by the DNA. In some cases, the molecule that signals deployment is an element of the hydrogel and is released from the device in controlled manner (e.g., temporally or spatially).

Cells (e.g., fibroblasts) contained in the hydrogel described herein promote regeneration of a tissue or organ (e.g., a wound) immediately adjacent to the material, or at some distant site.

The stiffness and elasticity of materials, such as the hydrogels described herein, are determined by applying a stress (e.g., oscillatory force) to the material and measuring the resulting displacement (i.e., strain). The stress and strain occur in phase in purely elastic materials, such that the response of one (stress or strain) occurs simultaneously with the other. In purely viscous materials, a phase difference is detected between stress and strain. The strain lags behind the stress by a 90 degree (radian) phase lag. Viscoelastic materials have behavior in between that of purely elastic and purely viscous—they exhibit some phase lag in strain. The storage modulus in viscoelastic solid materials are a measure of the stored energy, representing the elastic portion, while the loss modulus in viscoelastic solids measure the energy dissipated as heat, representing the viscous portion. The storage modulus represents the stiffness of a viscoelastic material and is proportional to the energy stored during a stress/displacement.

For example, the storage and loss moduli are described mathematically as follows:

Storage modulus:

$${E}^{′} = \frac{{σ}_{0}}{{ε}_{0}} cos δ$$

Loss modulus:

$${E}^{″} = \frac{{σ}_{0}}{{ε}_{0}} sin δ$$

### Phase Angle:

$$δ = arctan \frac{{E}^{″}}{{E}^{′}} ,$$

where stress is: σ=σ₀ sin(tω+δ),
strain is: ε=ε₀ sin(tω),
ω is frequency of strain oscillation, t is time, and δ is phase lag between stress and strain. See, e.g., Meyers and Chawla (1999) Mechanical Behavior of Materials. 98-103).

The storage modulus of a hydrogel is altered by varying the type of polymer used with alginate to form an IPN, e.g., type of collagen, or MATRIGEL™. In other examples, the storage modulus is altered by increasing or decreasing the molecular weight of the alginate. For example, the alginate is at least about 30 kDa, e.g., at least about 30, 40, 50, 60, 70, 80, 90, 100, 120, 140, 160, 180, 190, 200, 210, 220, 230, 240, 250, 260, 270, 280, 290, 300 kDa, or greater. For example, the molecular weight of the alginate is at least about 100 kDa, e.g., at least about 100, 120, 140, 160, 180, 190, 200, 210, 220, 230, 240, 250, 260, 270, 280, 290, 300 kDa, or greater. For example, the molecular weight of the alginate is about 200 kDa, 250 kDa, or 280 kDa. In other cases, the storage modulus is altered by increasing or decreasing the concentration of alginate, e.g., from about 1-15 mg/mL, or by increasing or decreasing the concentration of collagen/MATRIGEL™, e.g., from about 1-15 mg/mL. The storage modulus is also altered, e.g., by increasing or decreasing the type and concentration of cation used to crosslink the gel, e.g., by using a divalent versus trivalent ion, or by increasing or decreasing the concentration of the ion, e.g., from about 2-10 mM. In some cases, cation concentrations (e.g., Ca²⁺) of about 2-3 mM produce storage moduli of about 20-50 Pa, cation concentrations of about 4-5 mM produce storage moduli of about 200-300 Pa, cation concentration of about 7-8 mM produce storage moduli of about 300-600 Pa, and cation concentrations of about 9-10 mM produce storage moduli of about 1000-1200 Pa in hydrogels described herein, e.g., when storage moduli are measured at a frequency of 0.01 to 1 Hz, and e.g., when the concentration of alginate is about 5 mg/mL and the concentration of collagen is about 1.5 mg/mL, i.e., at a weight ratio of about 3.3 alginate to collagen.

In some examples, the hydrogel described herein is viscoelastic. For example, viscoelasticity is determined by using frequency dependent rheology. Collagen is a protein found in the extracellular matrix and is ubiquitously expressed in connective tissues. Collagens help tissues to withstand stretching. There are at least 16 types of collagen, and the most abundant type is Type I collagen (also called collagen-I). Collagen (e.g., collagen-I) is present in most tissues, primarily bone, tendon, and skin. The collagen molecules pack together, forming thin, long fibrils. Collagen (e.g., collagen I) is isolated, e.g., from rat tail. The fundamental structure of collagen-I is a long (˜300 nm) and thin (˜1.5 nm diameter) protein made up of three coiled subunits: two α1(I) chains and one α2(I). Each subunit contains 1050 amino acids and is wound around each other to form a right-handed triple helix structure. See, e.g., “Collagen: The Fibrous Proteins of the Matrix.” Molecular Cell Biology. Lodish et al., eds. New York: W.H. Freeman. Section 22.3 (2000); and Venturoni et al. Biochemical and Biophysical Research Communications 303 (2003) 508-513. The al chain of collagen-I has a molecular weight of about 140 kDa. The a2 chain of collagen-I has a molecular weight of about 130 kDa. Collagen-I as a trimer has a molecular weight of about 400 kDa. Collagen-I as a dimer has a molecular weigth of a bout 270 kDa. In some examples, the collagen in the hydrogels described herein include fibrillar collagen. Exemplary types of fibrillar collagen include collagen types I-III, V, XI, XXIV, and XXVII. See, e.g., Exposito, et al. Int. J. Mol. Sci. 11(2010):407-426.

The term, “about”, as used herein, refers to a stated value plus or minus another amount; thereby establishing a range of values. In certain preferred embodiments “about” indicates a range relative to a base (or core or reference) value or amount plus or minus up to 15%, 14%, 13%, 12%, 11%, 10%, 9%, 8%, 7%, 6%, 5%, 4%, 3%, 2%, 1%, 0.75%, 0.5%, 0.25% or 0.1%.

The following materials and methods were used in generating the data described in the Examples.

### Cell Culture

Human dermal fibroblasts (Zenbio) were cultured according to the manufacturer's protocol, and used between passages 6 and 11. For routine cell culture, cells were cultured in dermal fibroblasts culture medium (Zenbio), which contains specific growth factors necessary for optimal expansion of human dermal fibroblasts. Cells were maintained at sub-confluency in the incubator at 37° C. and 5% CO₂. The culture medium was refreshed every three days.

### Alginate Preparation

High molecular weight (LF20/40) sodium alginate was purchased from FMC Biopolymer. Alginate was dialyzed against deionized water for 2-3 days (molecular weight cutoff of 3,500 Da), treated with activated charcoal, sterile filtered (0.22 μm), lyophilized, and then reconstituted in DMEM serum free media at 2.5% wt.

### IPN Preparation

All inter-penetrating networks (IPNs) in this study consisted of 1.5 mg/ml rat-tail collagen-I (BD Biosciences), and 5 mg/ml high molecular weight alginate (FMC Biopolymer). The IPN matrix formation process consisted of two steps. In the first step, reconstituted alginate (2.5% wt in serum-free DMEM) was delivered into a centrifuge tube and put on ice. Rat-tail collagen-I was mixed with a 10×DMEM solution in a 1:10 ratio to the amount of collagen-I needed, pH was then adjusted to 7.4 using a 1M NaOH solution. The rat-tail collagen-I solution was thoroughly mixed with the alginate solution. Since the rat-tail collagen-I concentrations varied between batches, different amounts of DMEM were then added to the collagen-alginate mixture to achieve the final concentration of 1.5 mg/ml rat-tail collagen-I in the IPN. Once the collagen-alginate mixture was prepared, the human dermal fibroblasts were washed, trypsinized (0.05% trypsin/EDTA, Invitrogen), counted using a Z2 Coulter Counter (Beckman Coulter), and resuspended at a concentration of 3×10⁶ cells per ml in cell culture medium. Cells were mixed with the collagen-alginate mixture. The collagen-alginate-cells mixture was then transferred into a pre-cooled 1 ml luer lock syringe (Cole-Parmer).

In the second step, a solution containing calcium sulfate dihydrate (Sigma), used to crosslink the alginate network, was first prepared as follows. Calcium sulfate dihydrate was reconstituted in water at 1.22 M and autoclaved. For each IPN, 100 μl of DMEM containing the appropriate amount of the calcium sulfate slurry was added to a 1 ml luer lock syringe. The syringe with the calcium sulfate solution was agitated to mix the calcium sulfate uniformly, and then the two syringes were connected together with a female-female luer lock coupler (Value-plastics). The two solutions were mixed rapidly and immediately deposited into a well in a 48-well plate. The plate was then transferred to the incubator at 37° C. and 5% CO₂ for 60 minutes to allow gelation, after which medium was added to each gel. Medium was refreshed every two days.

### Scanning Electron Microscopy

For scanning electron microscopy, IPNs were fixed in 4% paraformaldehyde (PFA), washed several times in PBS, and serially transitioned from dH₂O into absolute ethanol with 30 min incubations in 30, 50, 70, 90, and 100% ethanol solutions. Ethanol dehydrated IPNs were dried in a critical point dryer and adhered onto sample stubs using carbon tape. Samples were sputter coated with 5 nm of platinum-palladium and imaged using secondary electron detection on a Carl Zeiss Supra 55 VP field emission scanning electron microscope (SEM).

### Elemental Analysis

For elemental analysis, IPNs were fixed in 4% paraformaldehyde (PFA), washed several times in PBS, quickly washed with dH₂O, froze overnight at −20° C. and lyophilized. Elemental analysis was performed using a Tescan Vega3 Scanning Electron Microscope (SEM) equipped with a Bruker Nano XFlash 5030 silicon drift detector Energy Dispersive Spectrometer (EDS).

### Mechanical Characterization of IPNs

The mechanical properties of the IPNs were characterized with an AR-G2 stress controlled rheometer (TA Instruments). IPNs without cells were formed as described above, and directly deposited onto the pre-cooled surface plate of the rheometer. A 20 mm plate was immediately brought into contact before the IPN started to gel, forming a 20 mm disk of IPN. The plate was warmed to 37° C., and the mechanical properties were then measured over time. The storage modulus at 0.5% strain and at 1 Hz was recorded every minute until it reached its equilibrium value (30-40 min). A strain sweep was performed to confirm that this value was within the linear elastic regime, followed by a frequency sweep.

### Analysis of Macromolecular Transport in IPNs

The diffusion coefficient of 70 kDa fluorescently labeled anionic dextran (Invitrogen) through IPNs used in this study (50 Pa-1200 Pa) was measured. For these studies, IPNs of varying mechanical properties encapsulating 0.2 mg/ml fluorescein-labeled dextran were prepared in a standard tissue culture 48 well-plate. IPNs were allowed to equilibrate at 37° C. for one hour, before serum-free phenol red-free medium was added to the well. Aliquots of this media were taken periodically to measure the molecular diffusion of dextran from the hydrogels into the media. Samples were continuously agitated using an orbital shaker, and fluorescein-labeled dextran concentration was measured using a fluorescence plate reader (Biotek). The measurements were interpreted using the semi-infinite slab approximation as described previously (Crank J. The mathematics of diffusion. 2nd Edition. Oxford University Press: Clarendon Press. 1979).

### Immunohistochemistry

The IPNs were fixed in 4% paraformaldehyde for 1 hour at room temperature and washed in PBS overnight at 4° C. The gels were embedded in 2.5% low gelling temperature agarose (Lonza) by placing the gels in liquid agarose in a 40° C. water bath for several hours and subsequent gelling at 4° C. A Leica vibratome was used to cut 200 μm sections. The F-actin cytoskeleton of embedded cells was visualized by probing sections with Alexa Fluor 488 conjugated Phalloidin (Invitrogen). Cell nuclei were stained with Hoechst 33342 (Invitrogen). To visualize the distribution of alginate within the IPN gels, gels were made using FITC-labeled alginate. To visualize the distribution of collagen-I fibers within the IPN gels, the collagen meshwork was probed with a rabbit anti-collagen-I polyclonal antibody (Abcam) and stained with an Alexa Fluor 647 conjugated goat-anti-rabbit IgG, after vibratome sectioning. Fluorescent micrographs were acquired using an Upright Zeiss LSM 710 confocal microscope.

### Cell Retrieval for Gene Expression and Flow Cytometry Analysis.

To retrieve the fibroblasts encapsulated within the IPN, the culture media was first removed from the well and the IPNs were washed once with PBS. Next the IPNs were transferred into a falcon tube containing 10 ml of 50 mM EDTA in PBS in which they remained for 30 minutes on ice. The resulting solution was then centrifuged and the supernatant removed. The remaining gel pieces were then incubated with a solution of 500 U/mL Collagenase type IV (Worthington) in serum free medium for 30 minutes at 37° C. and 5% CO₂, vigorously shaking to help disassociate the gels. The resulting solution was then centrifuged and the enzyme solution removed. The cell pellet was immediately placed on ice.

For RNA expression analysis, the retrieved cells were then lysed using Trizol, and RNA was extracted following the manufacturer's guidelines (Life Technologies). For flow cytometry, the cell pellet was further filtered through a 40 μm cell strainer and then analyzed using a using a BD LSR II flow cytometer instrument. A monoclonal anti-human COX2 antibody (clone AS66, abcam) was used, followed by an Alexa Fluor 647 conjugated goat-anti-mouse IgG secondary antibody (LifeTechnologies).

qPCR

RNA was quantified using a NanoDrop ND-1000 Spectrophotometer. Reverse transcription was carried out with the RT2 First Strand Kit from Qiagen, 200 ng of total RNA were used per sample. The expression profile of a panel of genes was assessed with the Human Wound Healing PCR Array from Qiagen, on a 96-well plate format and using an ABI7900HT thermocycler from Applied Biosystems.

### ELISA

Cell supernatant was collected and analyzed for IL-10 using ELISA (eBioscience 88-7106) according to manufacturer's directions. Briefly, high binding 96-well plates (Costar 2592) were coated with anti-human IL-10 and subsequently blocked with BSA. IL-10 standards and supernatant were loaded and detected with biotin conjugated anti-human IL-10. At least 5 replicates were used for each condition.

### Wound Healing Materials

The materials described herein provide a new approach to aid and enhance wound healing for the treatment of chronic non-healing wounds. Diabetic ulcers, ischemia, infection and/or continued trauma contribute to the failure to heal and demand sophisticated wound care therapies. Using the IPNs described herein, the behavior of dermal fibroblasts can be controlled simply by tuning the storage moduli of a model wound dressing material containing such IPNs. The stiffness of the dressing materials can be designed to match the stiffness of an injured tissue based on site of injury, condition of the subject (e.g., type of injury), age of the subject. In addition to cutaneous wound healing, the materials described herein are useful for aiding wound healing in other tissues, e.g., bony, cartilaginous, soft, vascular, or mucosal tissue.

The wound dressing market is expanding rapidly and is estimated to be valued at $21.6 billion by 2018. Current developments in the field include wound dressing materials that incorporate antimicrobial, antibacterial, and anti-inflammatory agents. However, the importance of mechanical forces in the context of wound dressing design has been overlooked.

The material system described herein includes, e.g., an interpenetrating network (IPN) of two polymers (e.g., collagen and alginate) that are not covalently bonded but fully interconnected. Such IPNs allow for the decoupling of the effects of gel stiffness from gel architecture, porosity and adhesion ligand density. For example, both types of polymers used in the IPNs are biocompatible, biodegradable and widely used in the tissue engineering field. In some material systems, bulk stiffness can be controlled by increasing or decreasing the polymer concentration—however, this also changes the scaffold architecture and porosity. Other material systems permit the independent control of stiffness but lack a naturally occurring extracellular matrix element that is required to closely mimic the biological tissue microenvironment.

In some examples, the approach described herein is used in concert with biomaterial-based spatiotemporal control over the presentation of bioactive molecules, growth factor or cells, although use the gels in combination with bioactive molecules or cells is not required for an effect on wound healing. Wound dressing materials that significantly enhance the wound healing response are made by solely tuning the stiffness of a wound dressing material comprising the hydrogels described herein, e.g., without the addition of any other bioactive molecules, growth factors, or cells.

The invention will be further illustrated in the following non-limiting examples.

### Example 1: Calcium Crosslinking Controlled Gel Mechanical Properties Independent of Gel Structure

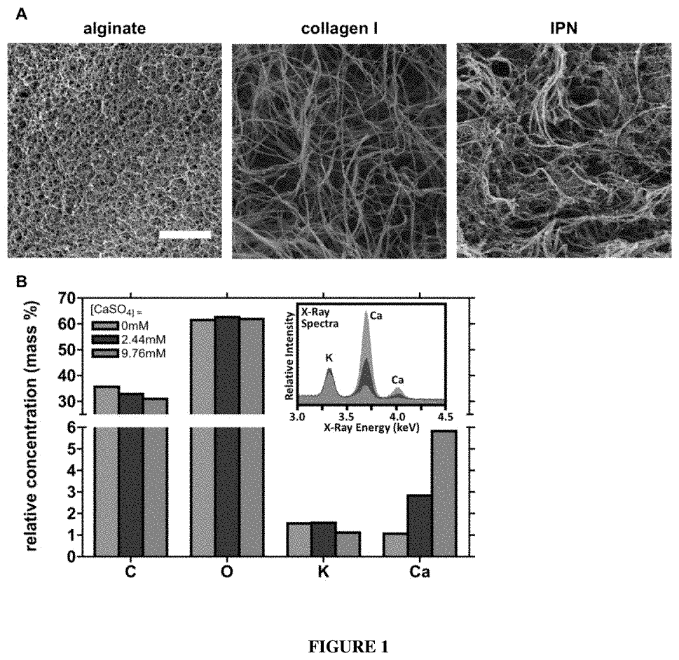

The microarchitecture of the alginate/collagen-I interpenetrating networks was assessed by scanning electron microscopy (SEM). SEM of hydrogels composed entirely of 0.5 mg/ml of alginate had an interconnected nanoporous scaffold structure (FIG. 1A). SEM of hydrogels composed entirely of 1.5 mg/ml collagen-I had a highly porous, randomly organized fribrillar network (FIG. 1A). SEM of the alginate/collagen-I interpenetrating networks had a true interpenetration of both components, with an interconnected nanoporous alginate mesh fully intercalated by multidirectional collagen-I fibrils (FIG. 1A). The dehydration and drying steps used to prepare the samples for SEM can cause shrinkage and consequent collapse of the porous structure of the hydrogels. However, since all samples were processed simultaneously and in the same fashion, these effects were expected to be similar across the different conditions analyzed.

The alginate network was crosslinked by divalent cations, such as calcium (Ca⁺²) that preferentially intercalate between the guluronic acid residues (“G-blocks”). Elemental mapping analysis of alginate/collagen-I interpenetrating networks, crosslinked to different extents with Ca⁺², confirmed that different amounts of calcium were present inside the interpenetrating network (FIG. 1B). The amount of calcium detected in the sample for which the alginate network was not crosslinked was likely due to residual amounts of calcium ions present in the culture media in which the hydrogels were immersed to equilibrate overnight.

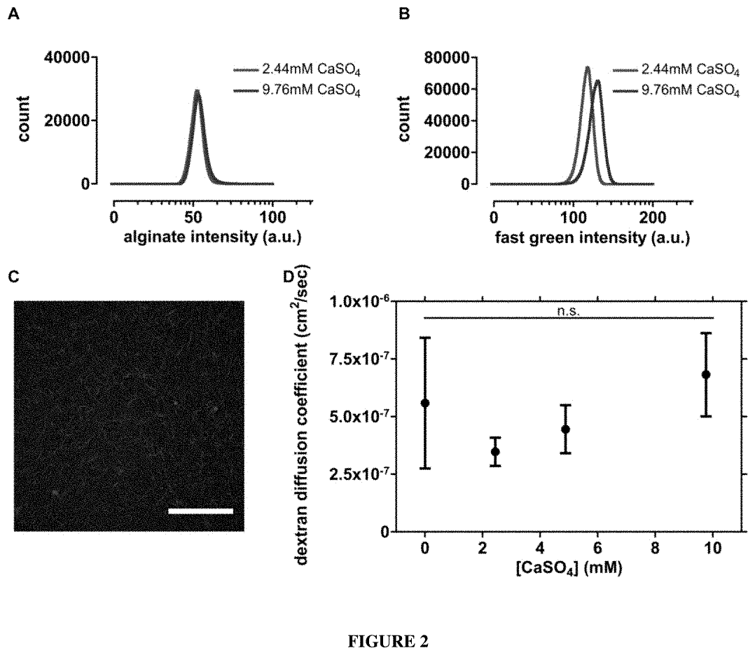

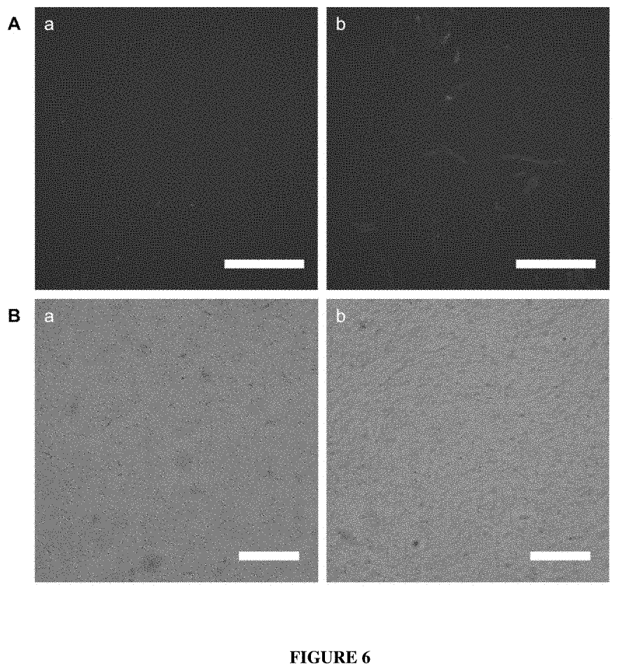

To establish the microscale distribution of the alginate chains within the interpenetrating networks, FITC-labeled alginate mixed with unlabeled collagen-I was visualized. In order to prevent any disruption on the architecture of the alginate mesh, the hydrogels were not washed, fixed or sectioned, but rather imaged directly after one hour of gelation at 37° C. The mixture of the two components showed no microscale phase separation independently of the extent of calcium crosslinking (FIGS. 2A and 6A), as shown on the histogram of fluorescent alginate intensity per pixel. Furthermore, FastGreen staining was used to visualize the protein content within the interpenetrating networks. Protein staining was uniform throughout the entire cross-section of these hydrogels, across the range of calcium crosslinking used (FIGS. 2B and 6B), as shown on the histogram of fast green intensity per pixel. A slight change in the peak location on the fast green intensity histogram was observed between the soft (crosslinked with 2.44 mM CaSO₄) and the stiff (crosslinked with 9.76 mM CaSO₄) samples, but the presence of only one peak in both samples indicated that there was an even distribution of the protein content along the hydrogel. Finally, a specific anti-collagen-I antibody staining was used to visualize the microarchitecture of the collagen network. Confocal fluorescence microscopy revealed a homogenous fribrillar mesh of collagen-I throughout the entire cross-section of the hydrogels, without any distinct patches of collagen-I (FIG. 2C). Thus, the networks were fully interpenetrating, independently of the degree of crosslinking of the alginate component.

To determine whether tuning the alginate crosslinking by varying the calcium concentration caused changes in gel pore size, macromolecular transport through the interpenetrating networks was analyzed. In particular, the diffusion coefficient of anionic high molecular weight dextran (70 kDa) through the various hydrogels was measured. No statistically significant differences in the diffusion coefficient of the dextran among the various interpenetrating networks of different stiffness were found (FIG. 2D), indicating that the pore size was constant as the concentration of calcium varied.

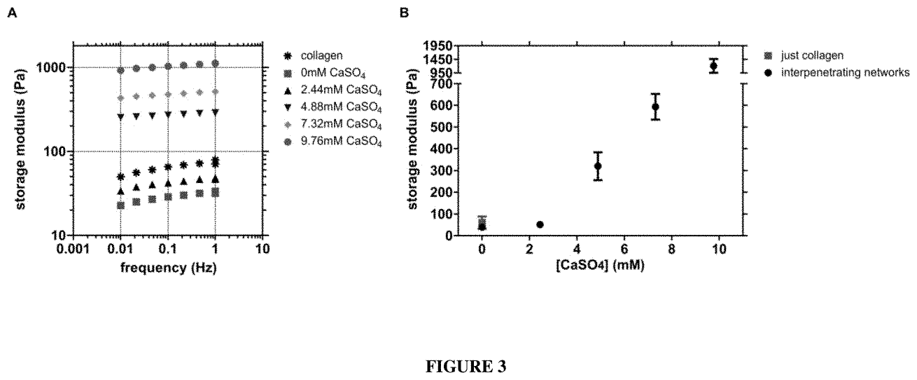

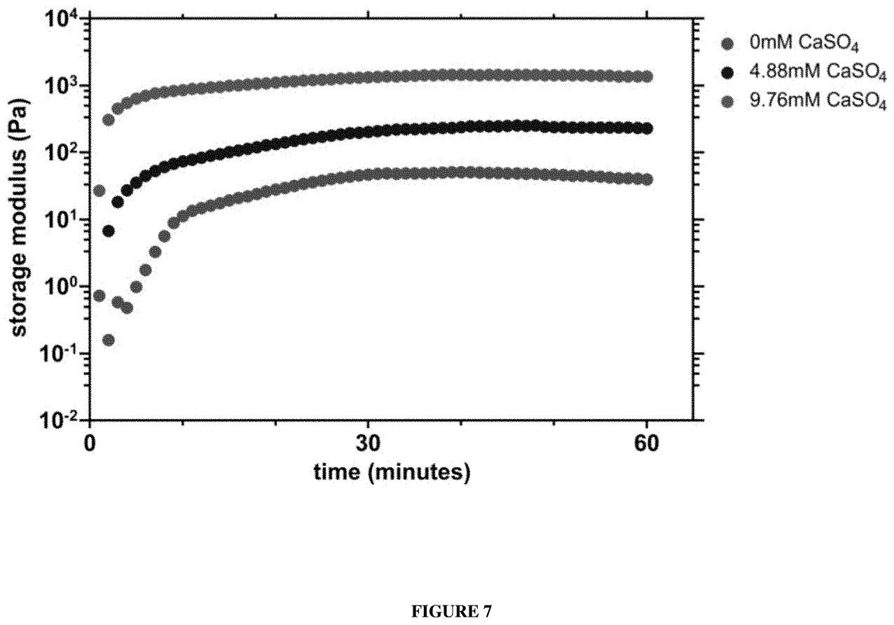

The mechanical properties of the alginate/collagen-I interpenetrating networks were assessed by rheology to determine if variations in calcium crosslinking would yield hydrogels with different moduli. The frequency dependent storage modulus of the different interpenetrating networks demonstrated that this biomaterial system exhibited stress relaxation, and that the viscoelastic behavior of these materials was independent of the extent of crosslinking (FIG. 3A). At a fixed frequency of 1 Hz across a time period of 60 minutes, the storage modulus was tuned from 50 to 1200 Pa by merely changing the initial concentration of calcium, while maintaining a constant polymer composition (FIG. 3B). The storage modulus of the pure collagen-I hydrogels was slightly higher than the alginate/collagen-I interpenetrating network with none or low amounts (2.44 mM) of CaSO₄, likely because the presence of the alginate chains plasticized the collagen-I network. The timecourse of gelation of the interpenetrating networks across a range of calcium crosslinker concentration was further assessed, and complete gelation of the matrices was achieved after 40-50 minutes at 37° C. (FIG. 7).

### Example 2: Fibroblasts Morphology Varied with IPN Moduli

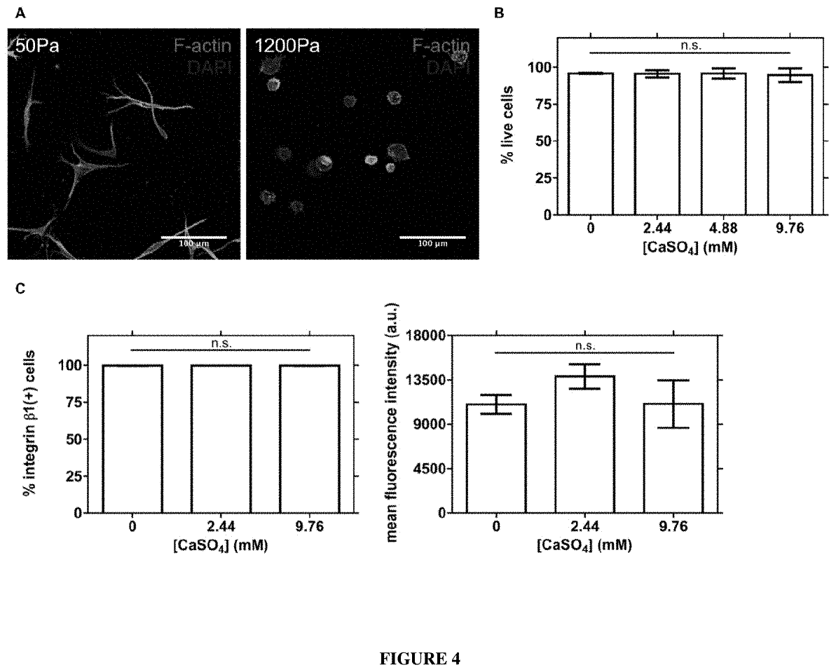

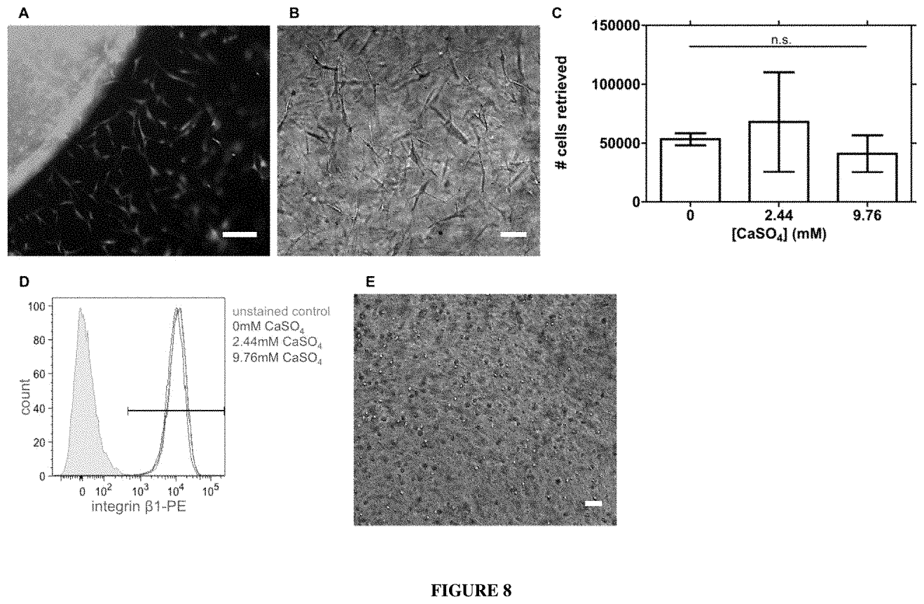

Human adult dermal fibroblasts isolated from the dermis of healthy non-diabetic donors were subsequently encapsulated within these alginate/collagen-I interpenetrating networks to examine the impact of gel mechanical properties on the cells' biology. Fibroblasts exhibited an elongated, spindle-like phenotype after a few hours of culture in the gels of lowest storage modulus (FIG. 4A). These softer matrices collapsed after a few days of culture, suggesting that the encapsulated cells were exerting traction forces on the matrix, contracting it and crawling out of hydrogel (FIG. 8A). In IPNs of increased stiffness, fibroblasts exhibited a spherical cell shape (FIG. 4A), up to at least 5 days of culture. Cells within these stiffer matrices failed to form stress fibers, as shown by confocal microscopy of F-actin staining of cryosections. These effects were not due to the higher concentrations of Ca⁺² in the stiffer interpenetrating networks, as when the highest amount of Ca⁺² (9.76 mM) was incorporated within hydrogels containing only collagen-I and dermal fibroblasts, cells were still able to spread and contract the matrix (FIG. 8B).

The fibroblasts encapsulated inside interpenetrating networks of different moduli were then retrieved and analyzed after 48 hours of culture. No statistically significant differences regarding cell number between matrices of different storage modulus were observed (FIG. 8C), and virtually all the cells encapsulated in interpenetrating networks of different moduli were alive after 48 hours of culture (FIG. 4B). As the attachment of primary fibroblasts to collagen type I is mediated by non-RGD-dependent β1 integrin matrix receptors (Jokinen et al. Journal of Biological Chemistry. 2004; 279:31956-63), flow cytometry measurements were used to analyze expression of this cell surface receptor. All the cells encapsulated in interpenetrating networks of different moduli expressed integrin (31 receptors, with no significant differences between their mean fluorescence intensity (FIGS. 4C and 8D).

To examine potential effects of altered cell adhesion ligand number in IPNs on the fibroblasts morphology, RGD cell adhesion motifs were coupled to the alginate prior to IPN formation. No differences in the phenotype of encapsulated fibroblasts between interpenetrating networks composed of unmodified and RGD-modified alginate chains were observed, independently of moduli tested (FIG. 8E).

### Example 3: Wound Healing-Related Genetic Programs Varied with IPN Moduli

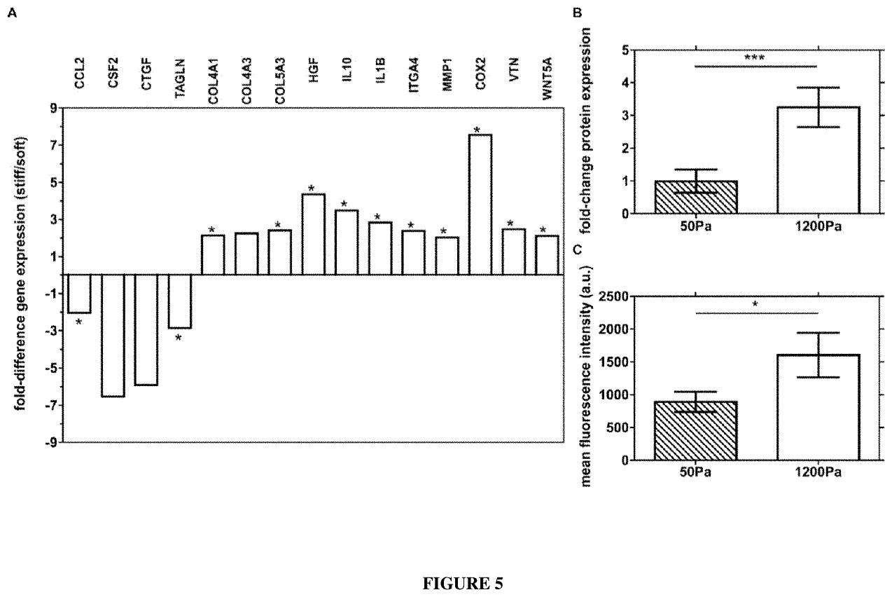

Experiments were performed to determine if the changes in cell spreading due to stiffness were accompanied by different gene expression profiles. Real-time reverse transcription polymerase chain reaction (RT-PCR) was used to analyze the expression of a panel of 84 genes important for each of the three phases of wound healing, including extracellular matrix remodeling factors, inflammatory cytokines and chemokines, as well as key growth factors and major signaling molecules. The gene screening revealed 15 genes displaying at least 2-fold difference in gene expression between dermal fibroblasts encapsulated in interpenetrating networks with storage moduli of 50 versus 1200 Pa (FIG. 5A). The expression of 11 genes was up-regulated in 1200 Pa versus 50 Pa gels, and expression of 4 genes was down-regulated in 1200 Pa versus 50 Pa gels. The genes which were down-regulated were chemokine ligand 2 (CCL2), colony stimulating factor 2 (CSF2), connective tissue growth factor (CTGF) and transgelin (TAGLN). A subset of three of the up-regulated genes is known to be involved in inflammation cascades: interleukin 10 (IL10), interleukin 1β (ILB1), and prostaglandin-endoperoxide synthase 2 (PTGS2) also known as COX2. A subset of collagen encoding genes was also up-regulated: collagen type IV, alpha 1 (COL4A1), collagen type IV, alpha 3 (COL4A3) and collagen type V, alpha 3 (COL5A3). Another subset of up-regulated genes represents cell adhesion and extracellular matrix molecules: integrin a4 (ITGA4), matrix metallopeptidase 1 (MMP1) and vitronectin (VTN). The remaining up-regulated genes were hepatocyte growth factor (HGF) and a member of the WNT gene family (WNTSA).

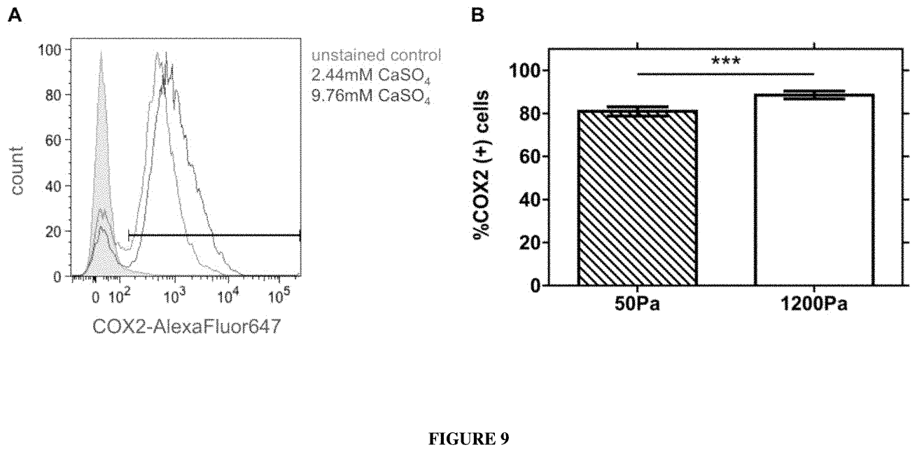

To validate the gene expression results, protein expression for IL10 and COX2 was analyzed. The amount of IL10 protein secreted into the culture medium by dermal fibroblasts encapsulated in interpenetrating networks of different storage modulus was measured by enzyme linked immunoassay (ELISA) (FIG. 5B), and enhanced matrix stiffness promoted a 3-fold increase in the production and secretion of this anti-inflammatory cytokine. Stiffening of the matrix also led to an increase in the number of cells expressing COX2 (FIGS. 4B and 9A) and an increase in the expression level in the cells staining positive for this inflammation-associated enzyme (FIG. 5C).

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A 3-dimensional hydrogel comprising an interpenetrating network of alginate and collagen, wherein the hydrogel comprises a storage modulus of 30 Pa or greater.

2. The hydrogel of claim 1, wherein the hydrogel comprises a storage modulus of 400 Pa or less.

3. The hydrogel of claim 1, wherein the alginate lacks a cell adhesion molecule.

4. The hydrogel of claim 3, wherein the cell adhesion molecule comprises a polypeptide comprising the amino acid sequence, arginine-glycine-aspartate (RGD).

5. The hydrogel of claim 1, wherein the hydrogel does not comprise any covalent crosslinks.

6. The hydrogel of claim 1, wherein the alginate is crosslinked to form a mesh structure.

7. The hydrogel of claim 6, wherein the alginate is ionically crosslinked.

8. The hydrogel of claim 7, wherein the alginate is ionically crosslinked by divalent or trivalent cations.

9. The hydrogel of claim 8, wherein the divalent cation comprises Ca²⁺.

10. The hydrogel of claim 1, wherein the alginate comprises a molecular weight of at least 100 kDa.

11. The hydrogel of claim 1, wherein the hydrogel comprises a dextran diffusion coefficient of 2.5×10⁻⁷ to 1×10⁻⁶ cm²/s.

12. The hydrogel of claim 1, wherein the hydrogel comprises multidirectional collagen fibrils.

13. The hydrogel of claim 1, wherein the hydrogel comprises a collagen concentration of about 1.5 mg/mL.

14. The hydrogel of claim 1, wherein the hydrogel comprises an alginate concentration of about 5 mg/mL.

15. The hydrogel of claim 1, wherein the hydrogel comprises interconnected pores.

16-20. (canceled)

21. The hydrogel of claim 1, further comprising a mammalian cell.

22-26. (canceled)

27. A wound dressing material comprising the hydrogel of claim 1.

28. The wound dressing material of claim 27, further comprising an anti-microbial or anti-inflammatory agent.

29. A method of promoting tissue repair, tissue regeneration, or wound healing comprising administering the hydrogel of claim 1 to a subject in need thereof.

30-42. (canceled)
